(19) United States
(12) Patent Application Publication
(10) Pub. No.: US 2026/0364020 A1
(43) Pub. Date: Nov. 19, 2026

(54) ADVANTAGEOUS FIXED-DOSE COMBINATION TREATMENT FOR HCV INFECTION

(71) Applicant: Atea Pharmaceuticals, Inc., Boston, MA (US)

(72) Inventors: Adel Moussa, Burlington, MA (US); James Michael Burgess Evans, Sudbury, MA (US); Luke McMurtray, Arlington, MA (US); Sindhura Ganga, Hopkinton, MA (US); Alex Cong Hung Vo, Mills Creek, WA (US); Nancy Gail Berry Agrawal, Audubon, PA (US)

(73) Assignee: Atea Pharmaceuticals, Inc., Boston, MA (US)

(21) Appl. No.: 19/701,301

(22) Filed: Jun. 8, 2026

Related U.S. Application Data
(63) Continuation of application No. PCT/US2026/012199, filed on Jan. 22, 2026.
(60) Provisional application No. 63/748,316, filed on Jan. 22, 2025.

(51) Int. Cl. A61K 9/20; A61K 9/16; A61K 31/5365; A61K 31/7076

(52) U.S. Cl. CPC A61K 9/2077; A61K 9/1652; A61K 9/2009; A61K 9/2018; A61K 9/2027; A61K 9/2054; A61K 31/5365; A61K 31/7076

(57) ABSTRACT

An advantageous fixed-dose combination of bemnifosbuvir, ruzasvir, and certain excipients, as well as its uses to treat hepatitis C or a condition related to hepatitis C, as well as methods for the manufacture of the fixed-dose combination:

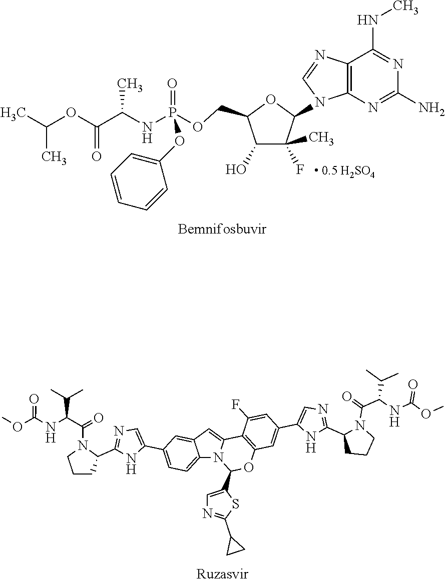

### CROSS REFERENCE TO RELATED APPLICATIONS

This application is a continuation of International Patent Application No. PCT/US2026/012199, filed Jan. 22, 2026, which claims the benefit of provisional U.S. Application No. 63/748,316 filed Jan. 22, 2025. The entirety of each of these applications is hereby incorporated by reference for all purposes.

### FIELD OF THE INVENTION

The present invention provides an advantageous therapeutic pharmaceutical combination formulation for the treatment of a human with a hepatitis C virus (HCV) infection that achieves multiple goals of therapy in one drug product.

### BACKGROUND OF THE INVENTION

Hepatitis C (HCV) is an RNA single-stranded virus and member of the Hepacivirus genus. A significant number of cases of liver disease are caused by HCV. HCV infection can lead to cirrhosis and liver cancer. If left to progress, liver cirrhosis and failure can require a liver transplant and can lead to death.

RNA polymerase is a key target that has been exploited to treat RNA single stranded viruses. The HCV non-structural protein NS5B RNA-dependent RNA polymerase is a key enzyme responsible for initiating and catalyzing viral RNA synthesis.

Examples of other HCV proteins that are additional targets for therapeutic approaches are NS3/4A (a serine protease) and NS5A (a non-structural protein that is an essential component of HCV replicase and exerts a range of effects on cellular pathways).

It is a strong challenge to provide an anti-HCV therapeutic that possesses multiple key properties simultaneously in a single drug product. These key properties are: (i) high antiviral activity; (ii) high activity against genotype 3 HCV: (iii) high drug exposure and bioavailability; (iv) avoids adverse effects against other drugs that the patient is taking (“drug-drug interactions”); (v) no food effect (i.e., the efficacy is not reduced if the drug product is taken with food); and (vi) minimization of the number of dosages required. In addition, and importantly, it is a major challenge to formulate a drug that is active against HCV that is classified as Class IV insoluble drug yet still simultaneously meets these requirements in the drug product.

It is an object of the present invention to provide an advantageous therapeutic pharmaceutical formulation drug product for the treatment of a human with a hepatitis C viral (HCV) infection that achieves these multiple goals of therapy in one drug product.

### SUMMARY OF THE INVENTION

An advantageous drug product formulation for the treatment of a human with a hepatitis C viral infection has been discovered that achieves the multiple goals of therapy in one drug product. This exceptional formulation exhibits: (i) high anti-HCV antiviral activity; (ii) high activity against genotype 3 HCV, which is historically challenging to treat; (iii) high drug exposure and bioavailability; (iv) avoidance of adverse effects with other drugs that the patient is taking (“drug-drug interactions”, which can lead to hepatotoxicity); (v) little or no food effect (i.e., the efficacy is not reduced if the drug product is taken with food); (vi) minimization of the number of dosages required; and (vii) successful administration of a difficult Class IV insoluble drug that is adequately stabilized in the formulation to achieve ample bioavailability.

The fixed-dose pharmaceutical is a coformulation of bemnifosbuvir (structure below), a highly active NS5B inhibitor of HCV, and ruzasvir (structure below), a highly active NS5A inhibitor. It was especially challenging to discover a formulation that includes the very active component ruzasvir that meets the above requirements. Ruzasvir is classified as a Class IV compound (low solubility and low cellular permeability) by the Biopharmaceutical Classification System (BCS). This typically presents a severe challenge to drug development. BCS Class IV compounds are commonly formulated with an efflux inhibitor to overcome their challenging physiochemical properties. However, efflux inhibitors present their own problems because they can disrupt the pharmacokinetics of other medications that a patient is taking, leading to potential liver toxicity. Liver toxicity is particularly harmful as a side effect of an HCV medication because the patient population already has impaired liver function. Therefore, ruzasvir has the challenge that if it is not administered with an efflux inhibitor, only a small proportion of drug gets into the cell. And if an efflux inhibitor is co-administered with the ruzasvir, it can adversely affect the cellular efflux of other drugs taken, leading to side effects.

The present invention advantageously allows ruzasvir to achieve its high anti-HCV potential without resorting to the use of an efflux inhibitor to achieve bioavailability. The present invention provides not only Class IV ruzasvir with high bioavailability, but also includes a second highly potent antiviral HCV compound, bemnifosbuvir.

### High Anti-HCV Antiviral Activity

The described specific anti-HCV coformulation contains the highly synergistic combination of bemnifosbuvir and ruzasvir. Bemnifosbuvir has high activity against all HCV genotypes, with EC95 values ranging from 10 to 25 nM depending on genotype. For comparison, the commercial anti-HCV compound sofosbuvir has EC₉₅ values that range from 50 to 265 nM depending on genotype.

Ruzasvir has shown picomolar potency against all HCV genotypes in preclinical studies (see, for example, Asante-Appiah, E. et al. 2018 In Vitro Antiviral Profile of Ruzsvir, a Potent and Pangenotype Inhibitor or Hepatitis C Virus NS5A, Antimicrob Agents Chemother 62:10.1128/aac.01280-18). The antiviral activity EC₅₀ values range from 1 to 4 picomolar, depending on genotype. These values were confirmed against clinical isolates available in public databases.

Synergy volumes were calculated from a three-dimensional plot of the antiviral activity of the compounds tested together at various concentrations compared to the antiviral activity of the compounds tested alone. Synergy volumes greater than 100 μM²% are considered “highly synergistic”. At 20 nM of bemnifosbuvir and 0.004 nM ruzasvir, the synergy volume was 103 μM²%. At 40 nM bemnifosbuvir and 0.008 nM ruzasvir, the synergy volume was 255 μM²%. Furthermore, at the concentrations tested, no synergistic toxicity was observed. The advantageous synergistic properties are described in WO 2022/266497.

### High Activity Against Genotype 3 HCV and Resistance Associated Variants

Genotype 3 is a historically difficult to treat genotype of hepatitis C. In a clinical trial, a surprising 100% of patients with genotype 3 HCV achieved sustained virologic response at 12 weeks (SVR12) when administered the combination of bemnifosbuvir and ruzasvir. The combination has an excellent clinical resistance profile and has been shown to overcome baseline pre-existing resistance associated substitutions. In fact, a patient with genotype 3 HCV and pre-existing nonstructural 5A protein resistance associated substitution S62A and Y93H achieved SVR12. The clinical results of the combination are described in Atea Pharmaceuticals “Lead-in Cohort Results From a Phase 2 Study of a Novel 8-Week Combination Regimen of Bemnifosbuvir and Ruzasvir in Patients with Chronic Hepatitis C Virus Infection” presented at European Association for the Study of the Liver Congress 2024

### High Drug Exposure and Bioavailability

As shown in Example 9, the specific fixed dose combination provides high exposure of both therapeutic compounds. The exposure of ruzasvir in the present formulation, including representative fixed dose formulations, is surprisingly high in a formulation that does not comprise an efflux inhibitor. In fact, the described formulation has approximately 150-200% of the exposure of certain other tested formulations.

Avoidance of Adverse Effects Against Other Drugs that the Patient is Taking

Vitamin E TPGS, an efflux inhibitor, has been shown to improve the bioavailability of ruzasvir. However, efflux inhibitors can cause drug-drug interactions leading to hepatotoxicity. Vitamin E TPGS is also a low melting solid, which is disadvantageous for manufacture and handling of solid dosage forms. As shown in the non-limiting illustrative Example 9, it has been surprisingly discovered that the described formulation, which contains no efflux inhibitors, enables a similar bioavailability as formulations that contain vitamin E TPGS. The described formulation not only has the same advantageous exposure as formulations with vitamin E TPGS, but also avoids potential hepatotoxicity.

### Does not Exhibit a Food Effect

It has been found that the described formulation does not exhibit a food effect. While a moderate food effect was observed in dogs (Example 10), in humans, no food effect was observed. It is beneficial for a formulation to have minimal or no food effect to ensure more predictable pharmacokinetics. In contrast to the present invention, a food effect was observed when patients were administered certain other formulations. Thus, the described specific coformulation advantageously does not have a food effect.

### Minimization of Pill Burden on Patients

Previous coformulations comprising ruzasvir used a loading of only approximately 20% in the spray dried dispersion. One of these coformulations was administered in two 1,500 mg tablets. The pill burden associated with such a large dosage form may impact patient compliance. Development of these combinations was discontinued. In contrast, the loading of ruzasvir in the spray dried dispersion of the coformulation described herein is more than double (50%, Example 7). This enables smaller dosage forms which reduces the pill burden on patients.

### Inclusion of a Class IV Insoluble Drug That Is Adequately Stabilized in the Formulation to Achieve Ample Bioavailability.

The disclosed fixed dose formulation surprisingly stabilizes ruzasvir. Selection of HPMC AS, and in particular, HPMC AS-L, promotes the formation of polymer-drug colloid species in the intestine. This increases the dissolution of ruzasvir and enables higher exposure (Example 8).

### Advantageous Fixed-Dose Combination

The advantageous fixed-dose combination of bemnifosbuvir and ruzasvir includes a compatible formulation of both drugs that does not include or require an efflux inhibitor. The fixed-dose combination combines bemnifosbuvir, a spray dried dispersion of ruzasvir, and other excipients, as described further herein, which are granulated together to form an intragranular mixture, which is then mixed with an extragranular material that holds the granulated material together. The intragranular/extragranular solid is then optionally covered with a protective coating.

In certain embodiments, ruzasvir is spray dried with hydroxypropylmethyl cellulose, which in a non-limiting illustrative embodiment is HPMC acetate succinate (HPMC AS, which can be HPMC AS L). HMPC AS L is a polymer that promotes colloidal formation in the intestine and improves solubilization of the drug. In certain embodiments, the ruzasvir loading in the spray dried dispersion is at least about 35% or even about 50%. The high loading of ruzasvir can decrease the pill burden for patients.

In certain embodiments the advantageous fixed-dose combination drug product comprises a mixture of bemnifosbuvir (optionally in a morphic form such as morphic form 3); a spray dried formulation of ruzasvir and HPMCAS (hydroxypropylmethylcellulose acetate succinate, for example in Grade L); and excipients that are rolled to form an intragranular material; additional extragranular material that assists in the integrity of the intragranular material and optionally a tablet coating.

In certain embodiments, the invention provides a fixed-dose combination drug product that comprises:
a) bemnifosbuvir optionally in a morphic form;
b) a spray-dried dispersion of ruzasvir, or a pharmaceutically acceptable salt thereof, in hydroxyalkylcellulose;
c) a mixture of
   i) a cellulosic polymer;
   ii) a sugar alcohol;
   iii) a disintegrant;
   iv) a glidant;
   v) a lubricant; and
   vi) an osmogen;
   vii) wherein the components are combined to create an intragranular mix;
d) combining the intragranular mix of c) with an extragranular material to form a solid material that holds the intragranular mix together; and
e) optionally coating with a tablet coating material to create the solid dosage form.

More specifically, the advantageous fixed-dose combination drug product comprises, or consists essentially of:
a) Bemnifosbuvir optionally in a morphic form, such as morphic form 3;
b) a spray-dried dispersion of ruzasvir, or a pharmaceutically acceptable salt thereof, in HPMCAS (hydroxypropylmethylcellulose acetate succinate, for example in Grade L);
c) a mixture of
   i) microcrystalline cellulose
   ii) mannitol
   iii) crospovidone,
   iv) a glidant such as silicon dioxide, which can be colloidal;
   v) a lubricant such as sodium stearyl fumarate; and
   vi) an osmogen, such as sodium chloride;
   vii) wherein the components are combined to create an intragranular mix;
d) combining the intragranular mix of vii) with an extragranular material to form a solid material that holds the intragranular mix together; and
e) optionally coating with a tablet coating material to create the solid dosage form.

In certain embodiments, this exceptional coformulation is prepared by the steps of
a) spray drying ruzasvir with HMPCAS;
b) mix spray dried dispersion of a) with bemnifosbuvir and silicon dioxide;
c) add mixture of a) and b) with other components;
d) roller compact the mix of c) to form the “intragranular material”;
e) mix the intragranular material of d) with an extragranular mixture of crospovidone, silicon dioxide (which may be colloidal) and sodium stearyl fumarate; and then
f) compress the mixture of f) into a solid dosage form, and
g) optionally coat the compressed mixture of f) with a tablet coating, for example Opadry® II.

This exceptional fixed-dose anti-HCV drug product provides:
(i) at least about 70% of the exposure of bemnifosbuvir and ruzasvir dosed individually,
(ii) does not contain an excipient that is an efflux inhibitor,
(iii) does not result in a food effect, and
(iv) results in at least about 85% dissolution of bemnifosbuvir and ruzasvir within 15 minutes.

### Bemnifosbuvir

Bemnifosbuvir (isopropyl ((S)-(((2R,3R,4R,5R)-5-(2-amino-6-(methylamino)-9H-purin-9-yl)-4-fluoro-3-hydroxy-4-methyltetrahydrofuran-2-yl) methoxy) (phenoxy)phosphoryl)-L-alaninate hemisulfate (AT-527)) is a highly potent HCV polymerase inhibitor.

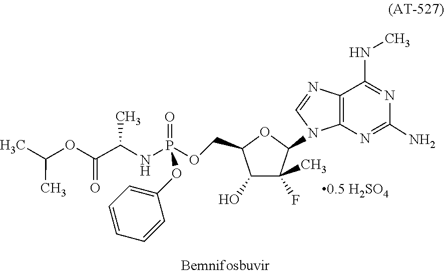 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F]

Bemnifosbuvir is the hemisulfate salt form of Compound 1, (isopropyl ((S)-(((2R,3R,4R,5R)-5-(2-amino-6-(methylamino)-9H-purin-9-yl)-4-fluoro-3-hydroxy-4-methyltetrahydrofuran-2-yl) methoxy) (phenoxy)phosphoryl)-L-alaninate. In a typical embodiment, Compound 1 is administered as the hemi-sulfate salt derivative (bemnifosbuvir).

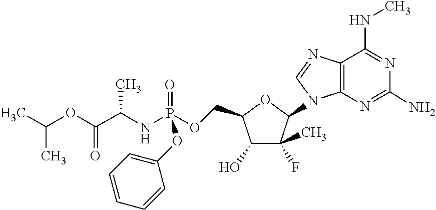 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F]

Bemnifosbuvir has high bioavailability, target organ selectivity, and high potency against all genotypes of HCV including genotype 3. Bemnifosbuvir as a monotherapy is typically administered in a dosage of 600 milligrams per day (which includes the weight of the salt, or 550 mg, if disregarding the weight of the salt), in two tablets. See generally U.S. Pat. Nos. 9,828,410; 10,000,523; 10,005,811; 10,239,911; 10,519,186; 10,815,266; 10,870,672; 10,870,673; 10,875,885; 10,894,804; and 10,906,928); and Berliba, E. et al. “Safety, Pharmacokinetics, and Antiviral Activity of AT-527, a Novel Purine Nucleotide Prodrug, in Hepatitis C Virus-Infected Subjects with or without Cirrhosis” 2019, Antimicrob Agents Chemother, 63 (12): e01201-19).

Atea Pharmaceuticals described in WO 2022/266497 the synergistic combination of bemnifosbuvir and ruzasvir for the treatment of HCV, which were initially provided as two separate orally administered tablets. The combination is currently being studied in two global Phase 3 clinical trials in patients with chronic HCV (NCT NCT06868264 and NCT07037277). These Phase 3 clinical trials will enroll over 1,500 patients worldwide. This invention achieves a fixed dose combination of the two components. This is the first report of an exceptional fixed dosage combination of the two drugs that simultaneously achieves the seven requirements listed above.

### Ruzasvir

Ruzasvir is a BCS Class IV oral, pan genotypic NS5A inhibitor that has been clinically evaluated for the treatment of chronic HCV infection. Ruzasvir has a sub- to low-picomolar affinity for HCV NS5A, across a wide range of HCV genotypes and common clinical mutants, including HCV genotype 1 with Y93H, Q30R, L31V, and Y93C mutations. (Tong et al. “Discovery of Ruzasvir (MK-8408): A Potent, Pan-Genotype HCV NS5A Inhibitor with Optimized Activity against Common Resistance-Associated Polymorphisms” J. Med. Chem. 2017, 60, 290-306).

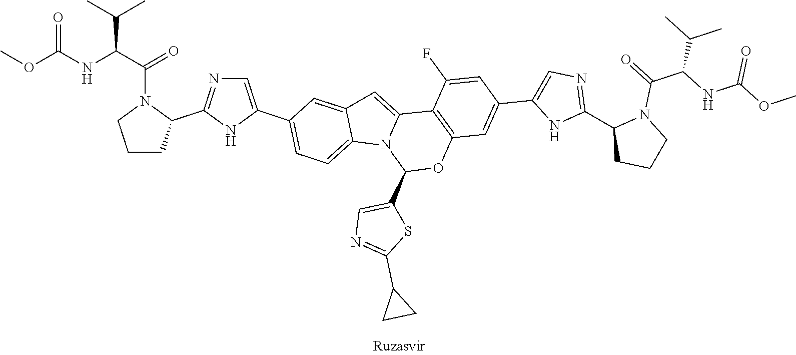

Despite the high, pan-genotypic potency of ruzasvir, its BCS class IV classification (i.e. it has both poor solubility and poor permeability) limits its use. A drug compound with both poor solubility and poor permeability is a formidable challenge for a monotherapy, and all the more so for a single dosage form combination therapy. BCS class IV compounds can present problems including erratic or poor absorption with inter- and intra-subject variability. BCS class IV compounds typically have low bioavailability. Adding to the challenge, BCS class IV compounds are commonly substrates for P-glycoprotein (P-gp), an efflux transporter, i.e., a protein that pumps the drug back out of the cell. Therefore, the small portion of the drug that manages to dissolve and get absorbed by the intestinal cells can be immediately shuttled back out into the digestive system and excreted.

For this reason, BCS class IV compounds are often co-administered with an efflux inhibitor. Efflux inhibitors minimize the transport by P-gp out of the cell. P-gp inhibition can assist BCS class IV compounds reaching therapeutically active levels, and somewhat overcome their problems with solubility and permeability. However, this strategy has significant drawbacks, in particular the chance of drug-drug interactions, i.e., an effect on other drugs the patient is taking. Due to the potential for drug-drug interactions and associated toxicity, it is strongly preferred not to have to co-administer a drug with a P-gp inhibitor (e.g vitamin E tocopherol polyethylene glycol succinate (TPGS)).

An example of this is treatments for people co-infected with HCV and HIV. Due to overlapping transmission routes, a substantial number of patients with HCV are coinfected with HIV. However, the HIV combination therapy Biktarvy® contains a compound which is a P-gp substrate (tenofovir alafenamide). If a patient who is taking Biktarvy® is administered a P-gp inhibitor (e.g. if their HCV therapy included an efflux inhibitor), the patient's plasma levels of tenofovir can rapidly increase, potentially resulting in serious liver and/or kidney toxicity. Because of this, the Biktarvy® label includes strong warnings about inhibition of P-gp and other drug-drug interactions. HCV already causes decreased liver function; thus, it is counterproductive to use an HCV therapeutic comprising an efflux inhibitor when that could cause additional liver damage.

Ruzasvir formulations have been previously prepared wherein ruzasvir was one component of a three-component combination. These formulations all used vitamin E TPGS (C-BREEZE-2 NCT: NCT02332707). Vitamin E TPGS is an efflux inhibitor, and therefore increases the exposure of ruzasvir when included in the formulation. However, as described above, using an efflux inhibitor to overcome the issues of BCS class IV compounds may result in dangerous drug-drug interactions and should be avoided.

After substantial research, a fixed dose combination of bemnifosbuvir and ruzasvir was discovered that does not contain an efflux inhibitor yet provides high exposure of ruzasvir. Surprisingly, this combination achieves nearly 50% higher levels of ruzasvir exposure than formulations containing efflux inhibitors (see AUC₀₋ₗₐₛₜ of FDC2A in comparison to AUC₀₋ₗₐₛₜ of FDC3 in Example 9). The fixed-dose combination of the present invention enables patients to receive all the benefits of ruzasvir without potentially exacerbating liver deterioration.

### Embodiments of the Advantageous Fixed-Dose Combination

The fixed dose combination pharmaceutical composition of the present invention provides high exposure through carefully selected components.

In certain embodiments, the secondary formulation comprises ruzasvir or a pharmaceutically acceptable salt thereof and hydroxypropyl methylcellulose acetate succinate (HPMCAS). In certain embodiments, the secondary formulation of ruzasvir comprises from about 40% to about 60% ruzasvir and from about 40% to about 60% HPMCAS, up to a total of 100%. In certain embodiments, the secondary formulation consists of 50% ruzasvir and 50% HPMCAS. In certain embodiments, the secondary formulation of ruzasvir and HPMCAS is a spray-dried dispersion.

In certain aspects, the advantageous fixed-dose combination comprises a lubricant, for example sodium stearyl fumarate, or alternatively sodium lauryl sulfate It was discovered as part of the invention that the addition of sodium lauryl sulfate to the spray-dried dispersion of ruzasvir can approximately double the exposure (Example 9).

In certain embodiments, bemnifosbuvir is provided in a crystalline form. In certain embodiments, bemnifosbuvir is provided in crystalline form 3, characterized by at least three, four, five, six, or seven XRPD peaks selected from 5.2±0.2°, 7.3±0.2°, 8.9±0.2°, 13.6±0.2°, 17.0±0.2°, 19.9±0.2°, and 21.8±0.2° 2theta (see Example 11) In one embodiment, this fixed-dose combination is intended to achieve a sustained viral response after administration for about or less than 12 weeks, for example about or less than 10 weeks, 8 weeks or 6 weeks or less.

In certain embodiments, this fixed dose co-formulated drug product is administered once a day for 8 weeks for non-cirrhotic HCV-infected patients and once a day for 12 weeks for cirrhotic patients. In each case, bemnifosbuvir is administered in a dosage of about 600 mg (about 550 mg of active nucleotide without salt weight) once a day and ruzasvir in a dosage of about 180 mg a day. In certain embodiments, this fixed dose co-formulated drug product is administered in a solid dosage form, for example a tablet, comprising about 300 mg bemnifosbuvir and about 90 mg ruzasvir. In certain embodiments, two tablets comprising about 300 mg bemnifosbuvir and about 90 mg ruzasvir are administered once per day.

The weight of active compound in the dosage form described herein is with respect to either the free form or the salt form of the compound unless otherwise specifically indicated.

In certain embodiments, the advantageous fixed-dose combination comprises between about 400 mg and about 650 mg of bemnifosbuvir, more typically between about 450 mg and about 600 mg, or between about 500 mg and about 600 mg. In certain embodiments, the advantageous fixed-dose combination comprises between about 120 mg and about 260 mg of ruzasvir, more typically between about 140 and about 220 mg, or between about 160 mg and about 200 mg.

In certain embodiments, the advantageous fixed-dose combination comprises between about 200 mg and about 375 mg of bemnifosbuvir, more typically between about 250 mg and about 325 mg. In certain embodiments, the advantageous fixed-dose combination comprises between about 60 mg and about 120 mg of ruzasvir, more typically between about 80 and about 100 mg.

The fixed-dose combination can be administered once, twice or three times or more a day, as recommended by the healthcare practitioner. In certain embodiments, the fixed-dose combination is provided once a day. In other embodiments, the fixed-dose combination is provided twice a day. In yet another embodiment, the fixed-dose combination is provided three times a day.

In certain embodiments, the fixed-dose combination comprises about 600 mg of bemnifosbuvir and about 180 mg of ruzasvir, which can be given 1, 2, or 3 times a day.

In certain embodiments, the fixed-dose combination comprises about 300 mg of bemnifosbuvir and about 90 mg of ruzasvir, which can be given 2, 3, or 4 times a day. In certain embodiments, the fixed-dose combination is administered in a dosage regimen of two dosage forms once per day. In certain embodiments, the fixed-dose combination is administered in a dosage regimen of two dosage forms twice per day. In certain embodiments, the fixed-dose combination is administered in a dosage regimen of two dosage forms three times per day.

In certain embodiments, the fixed-dose combination comprising bemnifosbuvir and ruzasvir is administered for up to 16 weeks, up to 12 weeks, for up to 10 weeks, for up to 8 weeks, for up to 6 weeks, or for up to 4 weeks. In alternative embodiments, the fixed-dose combination comprising bemnifosbuvir and ruzasvir is administered for at least 4 weeks, for at least 6 weeks, for at least 8 weeks, for at least 10 weeks, for at least 12 weeks, or for at least 24 weeks.

In certain embodiments, the fixed-dose combination comprising bemnifosbuvir and ruzasvir is administered at least once a day or every other day.

In certain embodiments, the fixed-dose combination comprising bemnifosbuvir and ruzasvir is administered twice a day. In certain embodiments, the fixed-dose combination comprising bemnifosbuvir and ruzasvir is administered three times a day.

The advantageous fixed-dose combination described herein can also be used to treat the range of HCV genotypes. At least six distinct genotypes of HCV, each of which have multiple subtypes, have been identified globally. Genotypes 1-3 are prevalent worldwide and Genotypes 4, 5, and 6 are more limited geographically. Genotype 4 is common in the Middle East and Africa. Genotype 5 is mostly found in South Africa. Genotype 6 predominately exists in Southeast Asia. Although the most common genotype in the United States is Genotype 1, defining the genotype and subtype can assist in treatment type and duration. For example, different genotypes respond differently to different medications. Optimal treatment times vary depending on the genotype infection. Within genotypes, subtypes, such as Genotype 1a and Genotype 1b, may respond differently to treatment as well. Infection with one type of genotype does not preclude a later infection with a different genotype.

In one embodiment, the fixed-dose combination comprising bemnifosbuvir and ruzasvir is used to treat HCV Genotype 1, HCV Genotype 2, HCV Genotype 3, HCV Genotype 4, HCV Genotype 5, or HCV Genotype 6. In one embodiment, the fixed-dose combination comprising bemnifosbuvir and ruzasvir is used to treat HCV Genotype 1a. In one embodiment, the fixed-dose combination comprising bemnifosbuvir and ruzasvir is used to treat HCV Genotype 1b. In one embodiment, the fixed-dose combination comprising bemnifosbuvir and ruzasvir is used to treat HCV Genotype 2a. In one embodiment, the fixed-dose combination comprising bemnifosbuvir and ruzasvir is used to treat HCV Genotype 2b. In one embodiment, the fixed-dose combination comprising bemnifosbuvir and ruzasvir is used to treat HCV Genotype 3a. In one embodiment, the fixed-dose combination comprising bemnifosbuvir and ruzasvir is used to treat HCV Genotype 3b. In one embodiment, the fixed-dose combination comprising bemnifosbuvir and ruzasvir is used to treat HCV Genotype 4a. In one embodiment, the fixed-dose combination comprising bemnifosbuvir and ruzasvir is used to treat HCV Genotype 4d. In one embodiment, the fixed-dose combination comprising bemnifosbuvir and ruzasvir is used to treat HCV Genotype 5a. In one embodiment, the fixed-dose combination comprising bemnifosbuvir and ruzasvir is used to treat HCV Genotype 6a. In one embodiment, the fixed-dose combination comprising bemnifosbuvir and ruzasvir is used to treat HCV Genotype 6b, 6c, 6d, 6e, 6f, 6g, 6 h, 6i, 6j, 6k, 6l, 6m, 6n, 6o, 6p, 6q, 6r, 6s, 6t, or 6u.

The invention also includes fixed-dose combinations and dosage forms wherein bemnifosbuvir may be in the form of an amorphous or crystalline salt and, independently, ruzasvir may be crystalline or amorphous.

The present invention thus includes at least the following aspects:
(a) An effective anti-HCV pharmaceutical composition comprising Compound 1, or a pharmaceutically acceptable salt thereof, and ruzasvir, and one or more pharmaceutically acceptable carriers selected from an enteric polymer, a filler, a disintegrant, a glidant, a lubricant, and an osmogen;
(b) An effective anti-HCV pharmaceutical composition comprising bemnifosbuvir and ruzasvir, and one or more pharmaceutically acceptable carriers selected from an enteric polymer, a filler, a disintegrant, a glidant, a lubricant, and an osmogen;
(c) The anti-HCV pharmaceutical composition of (a) or (b), wherein the pharmaceutical composition comprises an enteric polymer, a filler, a disintegrant, a glidant, a lubricant, and an osmogen;
(d) The anti-HCV pharmaceutical composition any one of (a)-(c), wherein the pharmaceutical composition comprises an intragranular mix and an extragranular mix, wherein the intragranular mix comprises:
   (i) Compound 1, or a pharmaceutically acceptable salt thereof, in a crystalline form;
   (ii) a spray-dried dispersion of ruzasvir and HPMCAS-L; and
   (iii) a filler, a disintegrant, a glidant, a lubricant, and an osmogen, as described herein;
   and the extragranular mix comprises one or more excipients selected from a disintegrant, a glidant, and a lubricant;
(e) The anti-HCV pharmaceutical composition of any one of (a)-(d), wherein the pharmaceutical composition comprises a ductile filler and a brittle filler;
(f) The anti-HCV pharmaceutical composition of any one of (a)-(e), wherein the enteric polymer is hydroxypropyl methylcellulose acetate succinate (HPMCAS);
(g) The anti-HCV pharmaceutical composition of any one of (a)-(f), comprising the ductile filler microcrystalline cellulose;
(h) The anti-HCV pharmaceutical composition of any one of (a)-(g), comprising the brittle filler mannitol;
(i) The anti-HCV pharmaceutical composition of any one of (a)-(h), wherein the disintegrant is crospovidone;
(j) The anti-HCV pharmaceutical composition of any one of (a)-(i), wherein the lubricant is sodium stearyl fumarate;
(k) The anti-HCV pharmaceutical composition of any one of (a)-(j), wherein the osmogen is sodium chloride;
(l) The anti-HCV pharmaceutical composition of any one of (a)-(k), wherein Compound 1 is bemnifosbuvir;
(m) The anti-HCV pharmaceutical composition of any one of (a)-(l), in the form of a pill, tablet, or capsule;
(n) The anti-HCV pharmaceutical composition of any one of (a)-(m), wherein the intragranular mix comprises:
   (i) from about 275 mg to about 325 mg of bemnifosbuvir;
   (ii) from about 160 mg to about 200 mg of a spray dried dispersion comprising about 50% ruzasvir and about 50% HPMCAS-L;
   (iii) from about 160 mg to about 180 mg of mannitol;
   (iv) from about 330 mg to about 360 mg of microcrystalline cellulose
   (v) from about 10 mg to about 15 mg of silicon dioxide;
   (vi) from about 80 mg to about 100 mg of crospovidone;
   (vii) from about 10 mg to about 15 mg of sodium stearyl fumarate; and
   (viii) from about 80 mg to about 100 mg of sodium chloride;
(o) The anti-HCV pharmaceutical composition of any one of (a)-(n), wherein the extragranular mix comprises:
   (i) from about 20 mg to about 30 mg of crospovidone;
   (ii) from about 4 mg to about 8 mg of silicon dioxide; and
   (iii) from about 4 mg to about 8 mg of sodium stearyl fumarate;
(p) The anti-HCV pharmaceutical composition of any one of (m)-(o), in the form of a tablet;
(q) The anti-HCV pharmaceutical composition of any one of (m)-(o), wherein the tablet is film coated;
(r) Use of an effective amount of the anti-HCV pharmaceutical composition of any one of (a)-(q) in the treatment of a hepatitis C virus infection in a human patient in need thereof;
(s) Use of an effective amount of the anti-HCV pharmaceutical composition of any one of (a)-(q) in the manufacture of a medicament for treatment of a hepatitis C virus infection in a human patient in need thereof;
(t) The anti-HCV pharmaceutical composition of any one of (a)-(q), for use in the treatment of a hepatitis C virus infection in a human patient in need thereof;
(u) A method for treating a hepatitis C virus infection comprising administering to a human patient in need thereof an effective amount of the anti-HCV pharmaceutical composition of any one of (a)-(q);
(v) Any of embodiments (r)-(u), wherein the HCV infection is genotype 1;
(w) Any of embodiments (r)-(u), wherein the HCV infection is genotype 2;
(x) Any of embodiments (r)-(u), wherein the HCV infection is genotype 3;
(y) Any of embodiments (r)-(u), wherein the HCV infection is genotype 4;
(z) Any of embodiments (r)-(u), wherein the HCV infection is genotype 5; and
(aa) Any of embodiments (r)-(u), wherein the HCV infection is genotype 6.

In the present invention, bemnifosbuvir can be provided in the advantageous pharmaceutical composition as a crystalline form, or alternatively, a crystalline form can be used in a spray dry manufacturing procedure.

### BRIEF DESCRIPTION OF THE FIGURES

FIG. 1 is a process diagram depicting a non-limiting exemplary process for the manufacture of an advantageous pharmaceutical composition of the pending claims (Example 7).

FIG. 2 is a dissolution plot of the advantageous pharmaceutical composition of the pending claims in 10 mM phosphate buffer, pH 6.5, with 1% SLS (Example 8).

FIG. 3 is an XRPD pattern of wet bemnifosbuvir Form III. The peaks are listed in Table 1 of Example 11.

FIG. 4 is an XRPD pattern of bemnifosbuvir Form III that has been dried. The peaks are listed in Table 2 of Example 11.

FIG. 5 is pharmacokinetics plot depicting the concentration of bemnifosbuvir over time as described in Example 12.

FIG. 6 is pharmacokinetics plot depicting the concentration of Compound 1-2 over time as described in Example 12.

FIG. 7 is pharmacokinetics plot depicting the concentration of Compound 1-7 over time as described in Example 12.

FIG. 8 is pharmacokinetics plot depicting the concentration of Compound 1-8 over time as described in Example 12.

FIG. 9 is pharmacokinetics plot depicting the concentration of ruzasvir over time as described in Example 12.

### DETAILED DESCRIPTION OF THE INVENTION

The present invention provides an advantageous fixed-dose combination of bemnifosbuvir and ruzasvir for the treatment of a hepatitis C infection in a human.

An advantageous therapeutic drug product formulation for the treatment of a human with a hepatitis C viral infection has been discovered that achieves the seven multiple goals of therapy in one drug product. This exceptional formulation exhibits:
(i) high anti-HCV antiviral activity;
(ii) high activity against genotype 3 HCV and resistance associated variants:
(iii) high drug exposure and bioavailability;
(iv) avoids adverse effects against other drugs that the patient is taking (“drug-drug interactions”, which can lead to hepatotoxicity;
(v) does not exhibit a negative food effect (i.e., the bioavailability is not reduced if the drug product is taken with food);
(vi) minimizes the pill burden on patients; and
(vii) includes a Class IV insoluble drug that is adequately stabilized in the formulation to achieve ample bioavailability.

### High Anti-HCV Antiviral Activity

The described specific coformulation contains the highly synergistic combination of bemnifosbuvir and ruzasvir. Bemnifosbuvir has high activity against all HCV genotypes, with EC₉₅ values ranging from 10 to 25 nM depending on genotype. For comparison, the commercial anti-HCV compound sofosbuvir has EC₉₅ values that range from 50 to 265 nM depending on genotype.

Ruzasvir has shown picomolar potency against all HCV genotypes in preclinical studies (see, for example, Asante-Appiah, E. et al. 2018 In Vitro Antiviral Profile of Ruzsvir, a Potent and Pangenotype Inhibitor or Hepatitis C Virus NS5A, Antimicrob Agents Chemother 62:10.1128/aac.01280-18). The antiviral activity EC₅₀ values range from 1 to 4 picomolar, depending on genotype. These values were confirmed against clinical isolates available in public databases.

Synergy volumes can be calculated from a three-dimensional plot of the antiviral activity of the compounds tested together at various concentrations compared to the antiviral activity of the compounds tested alone. Synergy volumes greater than 100 μM²% are considered “highly synergistic”. A synergy volume of 103 μM²%, indicating a highly synergistic antiviral effect, was measured at 20 nM of bemnifosbuvir and 0.004 nM ruzasvir. An even higher synergy volume of 255 μM²% was measured at 40 nM bemnifosbuvir and 0.008 nM ruzasvir. Furthermore, at the concentrations tested, no synergistic toxicity was observed. The advantageous synergistic properties are described in WO 2022/266497.

### High Activity Against Genotype 3 HCV and Resistance Associated Variants:

Genotype 3 is a historically difficult to treat genotype of hepatitis C. Surprisingly, in a clinical trial, 100% of patients with genotype 3 HCV achieved sustained virologic response at 12 weeks (SVR12) when administered the combination of bemnifosbuvir and ruzasvir. The combination has an excellent clinical resistance profile and has been shown to overcome baseline pre-existing resistance associated substitutions. In fact, a patient with genotype 3 HCV and pre-existing nonstructural 5A protein resistance associated substitution S62A and Y93H achieved SVR12. The clinical results of the combination are described in Atea Pharmaceuticals “Lead-in Cohort Results From a Phase 2 Study of a Novel 8-Week Combination Regimen of Bemnifosbuvir and Ruzasvir in Patients with Chronic Hepatitis C Virus Infection” presented at European Association for the Study of the Liver Congress 2024

### High Drug Exposure and Bioavailability

As shown in Example 9, the specific fixed dose combination provides high exposure of both therapeutic compounds. The exposure of ruzasvir in the described formulation is surprisingly high in a formulation that does not comprise an efflux inhibitor. For example, the described formulation has approximately 150-200% of the exposure of certain other tested formulations.

Avoidance of Adverse Effects Against Other Drugs that the Patient is Taking

Vitamin E TPGS, an efflux inhibitor, has been shown to improve the bioavailability of ruzasvir. However, efflux inhibitors can cause drug-drug interactions leading to hepatotoxicity. Vitamin E TPGS is also a low melting solid, which is disadvantageous for manufacture and handling of solid dosage forms. As shown in Example 9, it has been surprisingly discovered that the described formulation, which contains no efflux inhibitors, enables a similar bioavailability as certain other formulations that contain vitamin E TPGS. The described formulation not only has the same advantageous exposure as formulations with vitamin E TPGS but also avoids potential hepatotoxicity.

### Does not Exhibit a Food Effect

It has been found that the described formulation does not exhibit a negative food effect. While a moderate food effect was observed in dogs (Example 10), in humans, there was no food effect observed. In contrast, a food effect was observed when patients were administered certain other formulations. Thus, the described specific coformulation advantageously does not have a food effect.

### Minimization of Pill Burden on Patients

Previous coformulations comprising ruzasvir used a loading of only approximately 20% in the spray dried dispersion. One of these coformulations was administered in two 1,500 mg tablets. The pill burden associated with such a large dosage form may impact patient compliance. Development of these combinations was discontinued. In contrast, the loading of ruzasvir in the spray dried dispersion of the coformulation described herein is more than double (50%, Example 7). This enables smaller dosage forms which reduces the pill burden on patients.

Includes a Class IV Insoluble Drug that is Adequately Stabilized in the Formulation to Achieve Ample Bioavailability.

Selection of HPMC AS, and in particular, HPMC AS-L, promotes the formation of polymer-drug colloid species in the intestine. This increases the dissolution of ruzasvir and enables higher exposure (Example 8).

### Fixed-Dose Combination

The anti-HCV compounds used in this fixed-dose combination therapy are: 1) the NS5B inhibitor isopropyl((S)-(((2R,3R,4R,5R)-5-(2-amino-6-(methylamino)-9H-purin-9-yl)-4-fluoro-3-hydroxy-4-methyltetrahydrofuran-2-yl)methoxy)(phenoxy)phosphoryl)-L-alaninate (Compound 1), or a pharmaceutically acceptable salt thereof; and 2) the NS5A inhibitor Ruzasvir (dimethyl N,N′-([(6S)-6-(2-cyclopropyl-1,3-thiazol-5-yl)-1-fluoro-6H-indolo[1,2-c][1,3]benzoxazine-3,10-diyl]bis{1H-imidazole-5,2-diyl-(2S)-pyrrolidine-2,1-diyl[(2S)-3-methyl-1-oxobutane-1,2-diyl]})dicarbamate) or a pharmaceutically acceptable salt thereof. In a typical embodiment, Compound 1 is administered as the hemi-sulfate salt derivative (bemnifosbuvir).

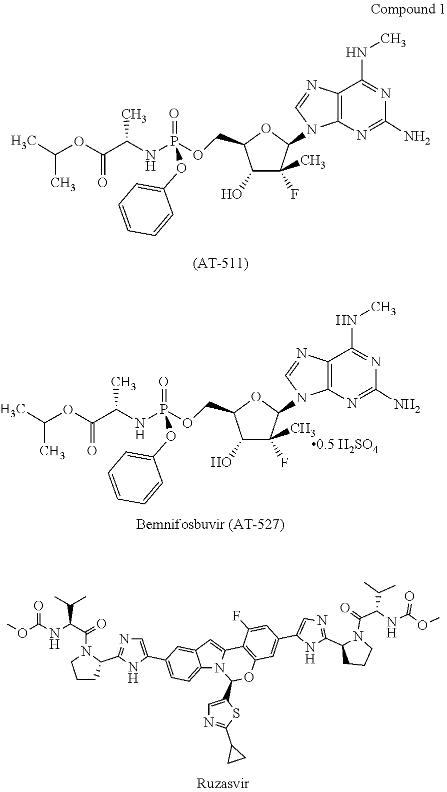

The advantageous fixed-dose combination of bemnifosbuvir and ruzasvir includes a compatible formulation of both drugs that does not include or require an efflux inhibitor. The fixed-dose combination combines bemnifosbuvir, a spray dried dispersion of ruzasvir, and other excipients, as described further herein, which are granulated together to form an intragranular mixture, which is then mixed with extragranular material that holds the granulated material together. The intragranular/extragranular solid is then optionally covered with a protective coating.

In certain embodiments, ruzasvir is spray dried with hydroxypropylmethyl cellulose, such as HPMC acetate succinate (HPMC AS, which can be HPMC AS L). HMPC AS L is a polymer that promotes colloidal formation in the intestine and improves solubilization of the drug. In certain embodiments, the ruzasvir loading in the spray dried dispersion is at least about 35% or even about 50%. The high loading of ruzasvir can decrease the pill burden for patients.

In certain embodiments the advantageous fixed-dose combination drug product comprises a mixture of bemnifosbuvir optionally in a morphic form such as morphic form 3; a spray dried formulation of ruzasvir and HPMCAS (hydroxypropylmethylcellulose acetate succinate, for example in Grade L); and excipients that are rolled to form an intragranular material; additional extragranular material that assists in the integrity of the intragranular material and optionally a tablet coating.

More specifically, the advantageous fixed-dose combination drug product comprises, or consists essentially of
a) Bemnifosbuvir optionally in a morphic form, such as morphic form 3;
b) a spray-dried dispersion of ruzasvir, or a pharmaceutically acceptable salt thereof, in hydroxyalkylcellulose, for example, HPMCAS (hydroxypropylmethylcellulose acetate succinate, for example in Grade L);
c) a mixture of
   i) a cellulosic polymer, for example, microcrystalline cellulose
   ii) a sugar alcohol, for example, mannitol
   iii) a disintegrant, such as crospovidone,
   iv) a glidant such as silicon dioxide, which can be colloidal;
   v) a lubricant such as sodium stearyl fumarate; and
   vi) an osmogen, such as sodium chloride;
   vii) wherein the components are combined to create an intragranular mix;
d) combining the intragranular mix of c) with an extragranular material to form a solid material that holds the intragranular mix together; and
e) optionally coating with a tablet coating material to create the solid dosage form.

In certain embodiments, this exceptional coformulation is prepared by the steps of
a) spray drying ruzasvir with H-MPCAS;
b) mixing the spray dried dispersion of a) with bemnifosbuvir and silicon dioxide;
c) adding the mixture of a) and b) with other components;
d) roller compacting the mix of c) to form the “intragranular material”;
e) mix the intragranular material of d) with an extragranular mixture of crospovidone, silicon dioxide (which may be colloidal) and sodium stearyl fumarate; and then
f) compress the mixture of f) into a solid dosage form, and
g) optionally coat the compressed mixture of f) with a tablet coating, for example Opadry® II.

This exceptional fixed-dose anti-HCV drug product provides:
(i) at least about 70% of the exposure of bemnifosbuvir and ruzasvir dosed individually,
(ii) does not contain an excipient that is an efflux inhibitor,
(iii) does not result in a food effect, and
(iv) results in at least about 85% dissolution of bemnifosbuvir and ruzasvir within 15 minutes.

### Hydroxyalkyl Cellulose

Hydroxyalkyl cellulose as described herein is a hydroxyalkyl ether of cellulose prepared by treating cellulose with sodium hydroxide and reacting with alkylene oxide. It is used as a water-binder and a thickening agent in pharmaceutical compositions to facilitate hydrophilization. Non-limiting examples of hydroxyalkyl celluloses include HPMCAS (hydroxypropylmethylcellulose acetate succinate hydroxymethylcellulose (HMC), hydroxyethyl cellulose (HEC), and hydroxypropyl methylcellulose (HPMC or hypromellose). Hypromellose (hydroxypropyl methylcellulose) is a non-ionic, partly O-methylated and O-(2-hydroxypropylated) cellulose ether derived semisynthetic polymer containing β-linked D-glucose.

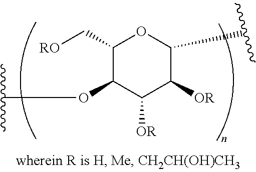

It is manufactured by reacting alkali cellulose, methyl chloride, and propylene oxide. It is available in various substitution ratios and molecular weight grades, and its HLB value ranges from 10 to 11. It is used as an emulsifier, hydrophilic thickening agent, and stabilizer due to film-foaming ability, biocompatibility, and biodegradability.

### Cellulosic Polymer

A cellulose polymer can be used as a dry binder, diluent, and disintegrant in solid oral dosage forms. Nonlimiting examples of a cellulosic polymer include microcrystalline cellulose, powdered cellulose, and hydroxypropyl cellulose. Microcrystalline cellulose (MCC) is a purified, partially depolymerized cellulose excipient obtained by controlled acid hydrolysis of α-cellulose. MCC forms dosage forms with reduced friability and improved uniformity, while its capillary action accelerates liquid uptake to facilitate tablet disintegration and dissolution. MCC is available in multiple particle size distributions and density grades to optimize flowability, compressibility, and segregation resistance and can be co-processed or surface-modified to tailor tensile strength and disintegration kinetics.

In certain embodiments, the MCC is a direct-compression grade (e.g., nominal bulk density ~0.25-0.35 g/mL). In certain embodiments, the MCC is a high-density grade (e.g., nominal bulk density ~0.40-0.55 g/mL). In certain embodiments, the MCC is a granular grade. In certain embodiments, the MCC is a colloidal/silicon-dioxide co-processed grade. In certain embodiments, the MCC is a silicified microcrystalline cellulose (SMCC) grade. In certain embodiments, the MCC is co-processed with lactose, mannitol, or starch.

### Sugar Alcohol

A sugar alcohol as used herein is a polyol derived from the hydrogenation of a sugar and contains one hydroxyl group (—OH) attached to each carbon atom. Sugar alcohols have the general formula HOCH₂(CHOH)ₙCH₂OH and exist in differing chain lengths, most commonly five- or six-carbon chains as they are derived from pentoses (five-carbon sugars) and hexoses (six-carbon sugars), respectively. They typically can be differentiated by the relative orientation (stereochemistry) of these —OH groups. For example, mannitol and sorbitol only differ in the orientation of hydroxyl group on carbon 2. Mannitol, as described herein, is a type of sugar alcohol derived from reduction of mannose, which produces sorbitol, a 2′-OH isomer of mannitol as the other product.

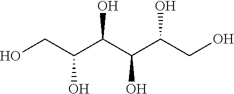 [SMILES: OC[C@@H](O)[C@@H](O)[C@H](O)[C@H](O)CO]

Sugar alcohols are inert and non-hygroscopic. Other sugar alcohols include but are not limited to erythritol, ethylene glycol, glycerol, threitol, arabitol, xylitol, ribitol, sorbitol, galactitol, fucitol, iditol, inositol, volemitol, maltitol and lactitol.

In certain embodiments, mannitol in the advantageous fixed-dose combination is replaced with an excipient such as sorbitol, xylitol, erythritol, anhydrous lactose, spray-dried lactose, or trehalose.

### Disintegrant

A disintegrant can be used in the advantageous fixed-dose combination described herein to improve the disaggregation of the solid dosage form into smaller particles for faster dissolution. Disintegrants can facilitate breakup of the solid dosage form by inducing swelling or through capillary wicking. Nonlimiting examples of disintegrants include crospovidone (crosslinked polyvinylpyrrolidone), croscarmellose sodium (crosslinked carboxymethyl cellulose), sodium starch glycolate (crosslinked carboxymethyl starch), low-substituted hydroxypropyl cellulose (L-HPC), pregelatinized starch, native corn starch, calcium silicate as a porous wicking agent, crosslinked alginates (e.g., sodium alginate-based systems), ion-exchange resins such as polacrilin potassium, and microcrystalline cellulose, which can contribute to disintegration through capillary wicking.

Crospovidone is a crosslinked polyvinylpyrrolidone used as a disintegrant to promote rapid breakup upon contact with aqueous media. Unlike swelling disintegrants, crospovidone primarily acts through capillary wicking and particle deformation, drawing fluid into the compact to disrupt interparticulate bonds without forming a gel. It can be effective at low loading and maintains disintegration performance across a wide pH range with minimal sensitivity to compression force.

### Glidant

A glidant improves the flowability of a powder which can assist in manufacturing processes such as tableting. Non-limiting examples of glidants include fatty acid salts (such as ascorbyl palmitate and calcium palmitate), magnesium stearate, magnesium carbonate, magnesium silicate, magnesium silicate hydroxide, magnesium oxide, powdered cellulose, fumed silica (colloidal silicon dioxide), starch and talc.

### Lubricant

Lubricants are excipients incorporated at low levels to reduce friction between the tablet mass and tooling during compression and ejection, thereby preventing sticking, picking, and mechanical wear. They function by forming a thin boundary layer on particle and die surfaces, lowering metal-powder adhesion and facilitating smooth ejection. Excessive lubrication can diminish tablet tensile strength and slow dissolution by impeding interparticle bonding. Common lubricants include fatty acids or fatty acid salts (such as stearic acid, myistic acid, palmitic acid, sodium stearyl fumarate, sodium lauryl sulfate, magnesium lauryl sulfate, sodium oleate, magnesium stearate, calcium stearate, beeswax, vegetable stearin, vegetable oil, hydrogenated vegetable oil, and hydrogenated castor oil), fatty acid esters (such as glyceryl behenate), sodium benzoate, sodium acetate, and inorganic polymers (such as talc, hydrated magnesium silicate, boric acid, PEG 4000).

### Osmogen

Osmogens (also known as an osmotic agent) are excipients that generate osmotic pressure by dissolving and creating a concentration gradient, thereby driving controlled influx of water into a dosage form and sustaining drug release. Non-limiting examples of osmogens include inorganic salts as well as soluble organic compounds such as sugars.

In certain embodiments, the osmogen is an inorganic salt, for example sodium chloride, potassium chloride, sodium sulfate, sodium phosphate tribasic, sodium phosphate dibasic, sodium phosphate, sodium bicarbonate, magnesium chloride, magnesium sulfate or mixtures thereof. In certain embodiments, the osmogen is an organic compound, for example mannitol, lactitol, sucrose, polyethylene glycol, lactose, fructose, dextrose, or mixtures thereof.

### Compound 1 and Bemnifosbuvir

Compound 1 (isopropyl((S)-(((2R,3R,4R,5R)-5-(2-amino-6-(methylamino)-9H-purin-9-yl)-4-fluoro-3-hydroxy-4-methyltetrahydrofuran-2-yl)methoxy)(phenoxy)phosphoryl)-L-alaninate) was previously described in U.S. Pat. Nos. 9,828,410; 10,000,523; 10,005,811; and 10,239,911 and PCT Applications WO 2016/21276 and WO 2019/200005 assigned to Atea Pharmaceuticals. The synthesis of Compound 1 is described in Example 1 below.

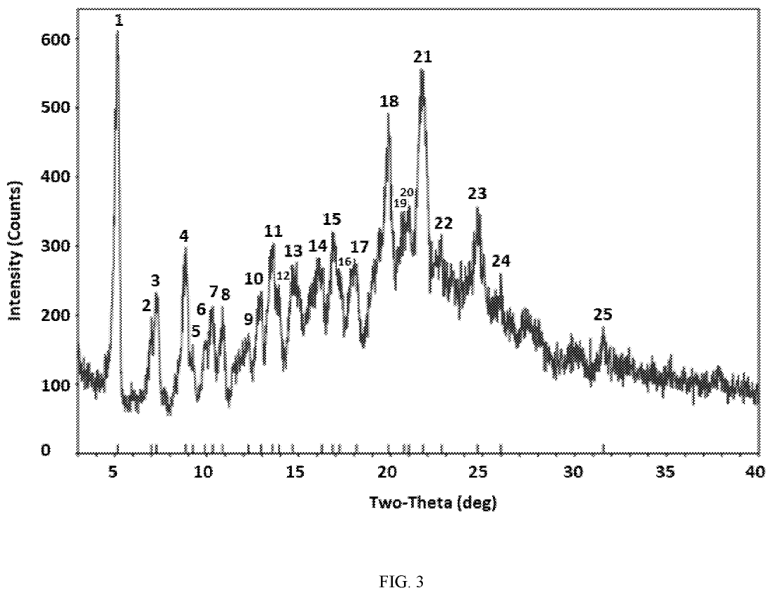

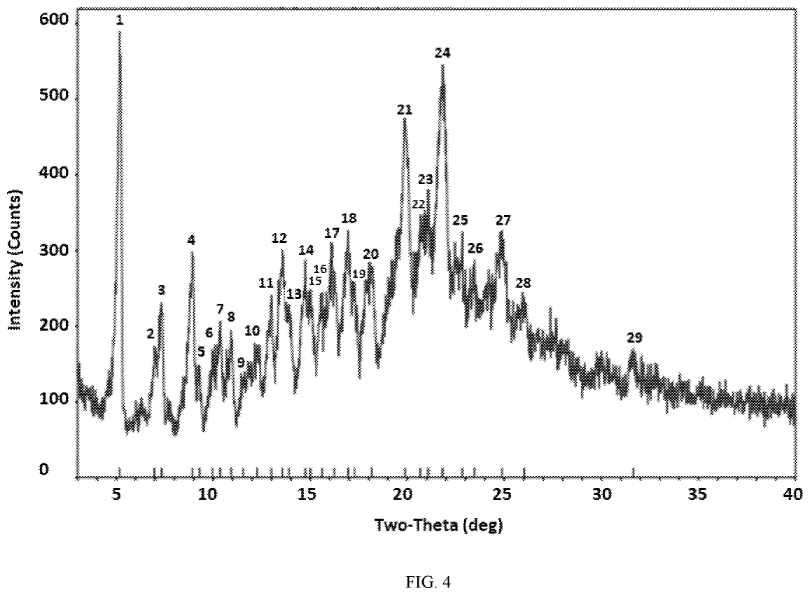

Bemnifosbuvir was previously disclosed in US 2018-0215776 and PCT Applications WO 2018/144640 and WO 2019/200005 assigned to Atea Pharmaceuticals. The synthesis of bemnifosbuvir (the hemi-sulfate salt of isopropyl((S)-(((2R,3R,4R,5R)-5-(2-amino-6-(methylamino)-9H-purin-9-yl)-4-fluoro-3-hydroxy-4-methyltetrahydrofuran-2-yl)methoxy)(phenoxy)phosphoryl)-L-alaninate) is described in Example 4 below. In one embodiment bemnifosbuvir is provided in a pharmaceutically acceptable composition or solid dosage form thereof. In another embodiment, bemnifosbuvir is an amorphous solid. In one embodiment, bemnifosbuvir is a crystalline solid. FIGS. 3-4 depict the characterization of a crystalline form of bemnifosbuvir by XRPD.

As described above, bemnifosbuvir successfully completed a Phase 1b/2a clinical trial for patients infected with HCV. The multiple part study evaluated the effect of single and multiple doses of bemnifosbuvir in healthy subjects, non-cirrhotic HCV-infected patients, and cirrhotic HCV-infected patients. Bemnifosbuvir induced significant antiviral reduction when administered to all HCV-infected cohorts tested. Bemnifosbuvir was administered once daily (QD) over the course of seven days, and potent antiviral activity was observed. In non-cirrhotic HCV-infected patients who were given 600 mg QD of bemnifosbuvir (equivalent to 550 mg of Compound 1), the mean maximum HCV RNA reduction was 4.4 log₁₀ IU/mL in HCV GT1-infected patients and 4.6 log₁₀ IU/mL in HCV GT3-infected patients. The effect of bemnifosbuvir on antiviral reduction also extended to the difficult-to-treat cirrhotic patients. In a cohort of HCV GT1 or HCV GT3-infected patients with CPA cirrhosis, the mean maximum HCV RNA reduction was 4.4 log₁₀ IU/mL when administered QD for seven days (Zhou, X. et al. “AT-527, a pan-genotypic purine nucleotide prodrug, exhibits potent antiviral activity in subjects with chronic hepatitis C” presented at The International Liver Congress 2018; Apr. 13, 2018; Paris, France).

Unless otherwise specified, Compound 1 or a pharmaceutically acceptable salt thereof is provided in the β-D-configuration. In an alternative embodiment, Compound 1 or a pharmaceutically acceptable salt thereof can be provided in a β-L-configuration. The phosphoramidate of Compound 1 or a pharmaceutically acceptable salt thereof can be provided as an R or S chiral phosphorus derivative or a mixture thereof, including a racemic or a diastereomeric mixture. All of the combinations of these stereoconfigurations are alternative embodiments in the invention described herein.

These alternative configurations include, but are not limited to:

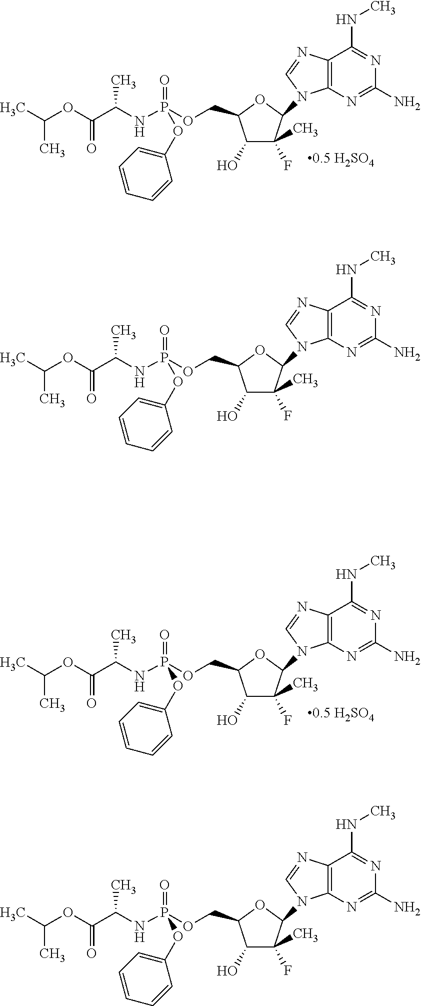 [SMILES: CNc1nc(N)nc2c1ncn2[C@@H]1O[C@H](COP(=O)(N[C@@H](C)C(=O)OC(C)C)Oc2ccccc2)[C@@H](O)[C@@]1(C)F ; CNc1nc(N)nc2c1ncn2[C@@H]1O[C@H](COP(=O)(N[C@@H](C)C(=O)OC(C)C)Oc2ccccc2)[C@@H](O)[C@@]1(C)F ; CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F ; CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F]

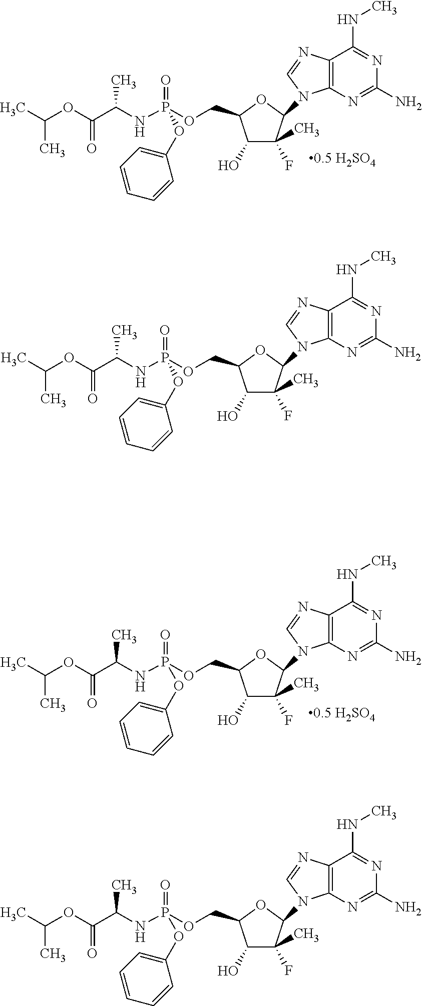 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F ; CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F ; CNc1nc(N)nc2c1ncn2[C@@H]1O[C@H](COP(=O)(N[C@H](C)C(=O)OC(C)C)Oc2ccccc2)[C@@H](O)[C@@]1(C)F ; CNc1nc(N)nc2c1ncn2[C@@H]1O[C@H](COP(=O)(N[C@H](C)C(=O)OC(C)C)Oc2ccccc2)[C@@H](O)[C@@]1(C)F]

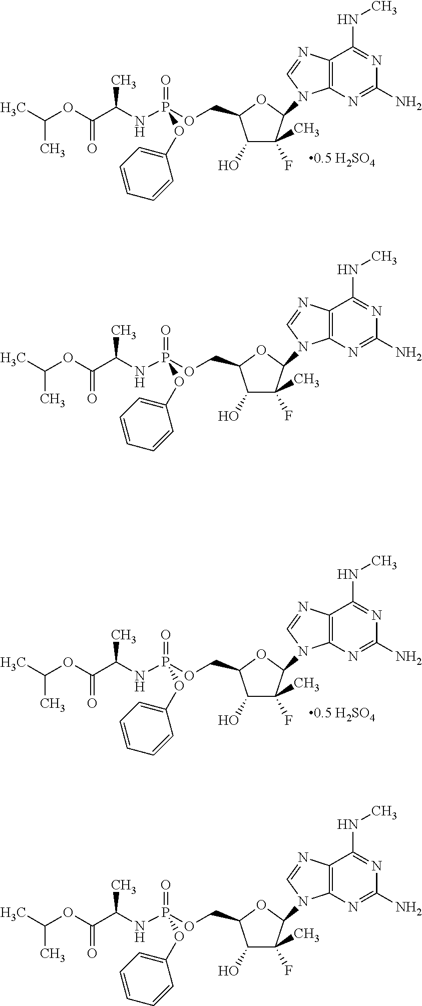 [SMILES: CNc1nc(N)nc2c1ncn2[C@@H]1O[C@H](CO[P@@](=O)(N[C@H](C)C(=O)OC(C)C)Oc2ccccc2)[C@@H](O)[C@@]1(C)F ; CNc1nc(N)nc2c1ncn2[C@@H]1O[C@H](CO[P@@](=O)(N[C@H](C)C(=O)OC(C)C)Oc2ccccc2)[C@@H](O)[C@@]1(C)F ; CNc1nc(N)nc2c1ncn2[C@@H]1O[C@H](CO[P@](=O)(N[C@H](C)C(=O)OC(C)C)Oc2ccccc2)[C@@H](O)[C@@]1(C)F ; CNc1nc(N)nc2c1ncn2[C@@H]1O[C@H](CO[P@](=O)(N[C@H](C)C(=O)OC(C)C)Oc2ccccc2)[C@@H](O)[C@@]1(C)F]

An additional alternative configuration includes

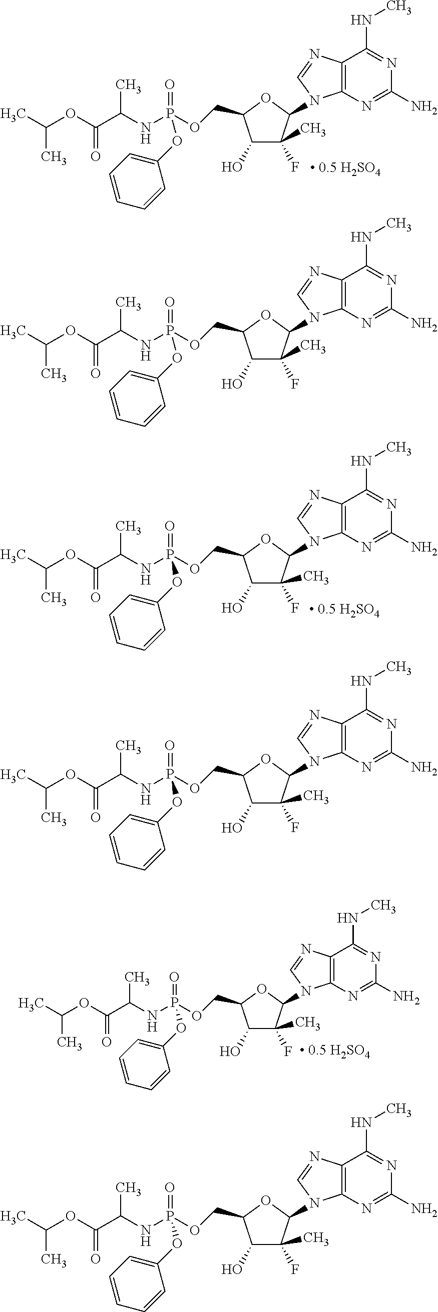

In embodiments, any of the above stereoisomers or a pharmaceutically acceptable salt thereof is used as an alternative to Compound 1, or a pharmaceutically acceptable salt thereof, in any aspect of the present invention herein.

In another alternative embodiment, Compound 1 is provided as the hemisulfate salt of a phosphoramidate other than the specific phosphoramidate described in the compound illustration. In yet another alternative embodiment, Compound 1 or a pharmaceutically acceptable salt thereof is provided as a phosphoramidate other than the specific phosphoramidate described in the compound illustration. A wide range of phosphoramidates are known to those skilled in the art which can be selected as desired to provide an active compound as described herein. For example, the phosphoramidate of Compound 1 or a pharmaceutically acceptable salt thereof in alternative embodiments includes a compound or pharmaceutically acceptable salt thereof of Formula A:

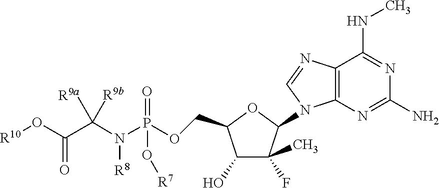

wherein:
R⁷ is C₁₋₆alkyl (including methyl, ethyl, propyl, and isopropyl), C₃₋₇cycloalkyl, or aryl (including phenyl and napthyl);
R⁸ is hydrogen or C₁₋₆alkyl (including methyl, ethyl, propyl, and isopropyl);
R^{9a} and R^{9b} are independently selected from hydrogen, C₁₋₆alkyl (including methyl, ethyl, propyl, and isopropyl), or C₃₋₇cycloalkyl; and
R¹⁰ is C₁₋₆alkyl (including methyl, ethyl, propyl, and isopropyl), C₁₋₆haloalkyl, or C₃₋₇cycloalkyl.

In alternative non-limiting embodiments, the present invention includes an alternative of Compound 1 as an oxalate salt (Compound 1-B), an HCl salt (Compound 1-C), or a sulfate salt (Compound 1-D).

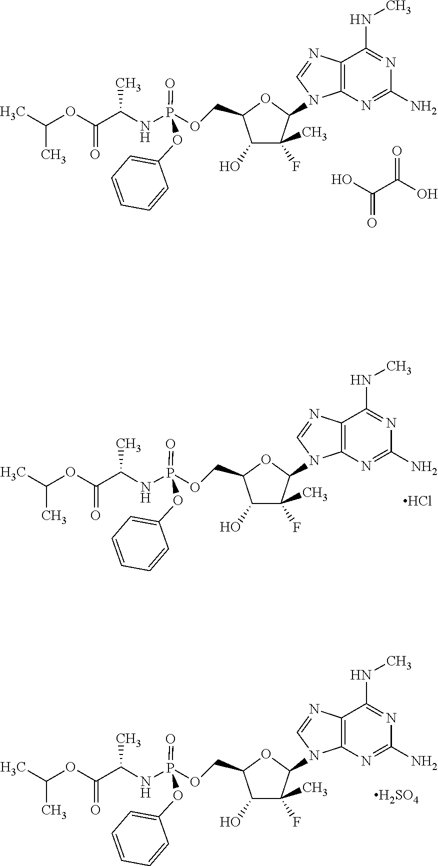

The metabolism of Compound 1 and bemnifosbuvir involves the production of a 5′-monophosphate and the subsequent anabolism of the N⁶-methyl-2,6-diaminopurine base (1-3) to generate ((2R,3R,4R,5R)-5-(2-amino-6-oxo-1,6-dihydro-9H-purin-9-yl)-4-fluoro-3-hydroxy-4-methyltetrahydrofuran-2-yl)methyl dihydrogen phosphate (1-4) as the 5′-monophosphate. The monophosphate is then further anabolized to the active triphosphate species: the 5′-triphosphate (1-6). The 5′-triphosphate can be further metabolized to generate 2-amino-9-((2R,3R,4R,5R)-3-fluoro-4-hydroxy-5-(hydroxymethyl)-3-methyltetrahydrofuran-2-yl)-1,9-dihydro-6H-purin-6-one (1-7).

Alternatively, 5′-monophophate 1-2 can be metabolized to generate the purine base 1-8. The metabolic pathway for isopropyl((S)-(((2R,3R,4R,5R)-5-(2-amino-6-(methylamino)-9H-purin-9-yl)-4-fluoro-3-hydroxy-4-methyltetrahydrofuran-2-yl)methoxy)(phenoxy)phosphoryl)-L-alaninate is illustrated in Scheme 1:

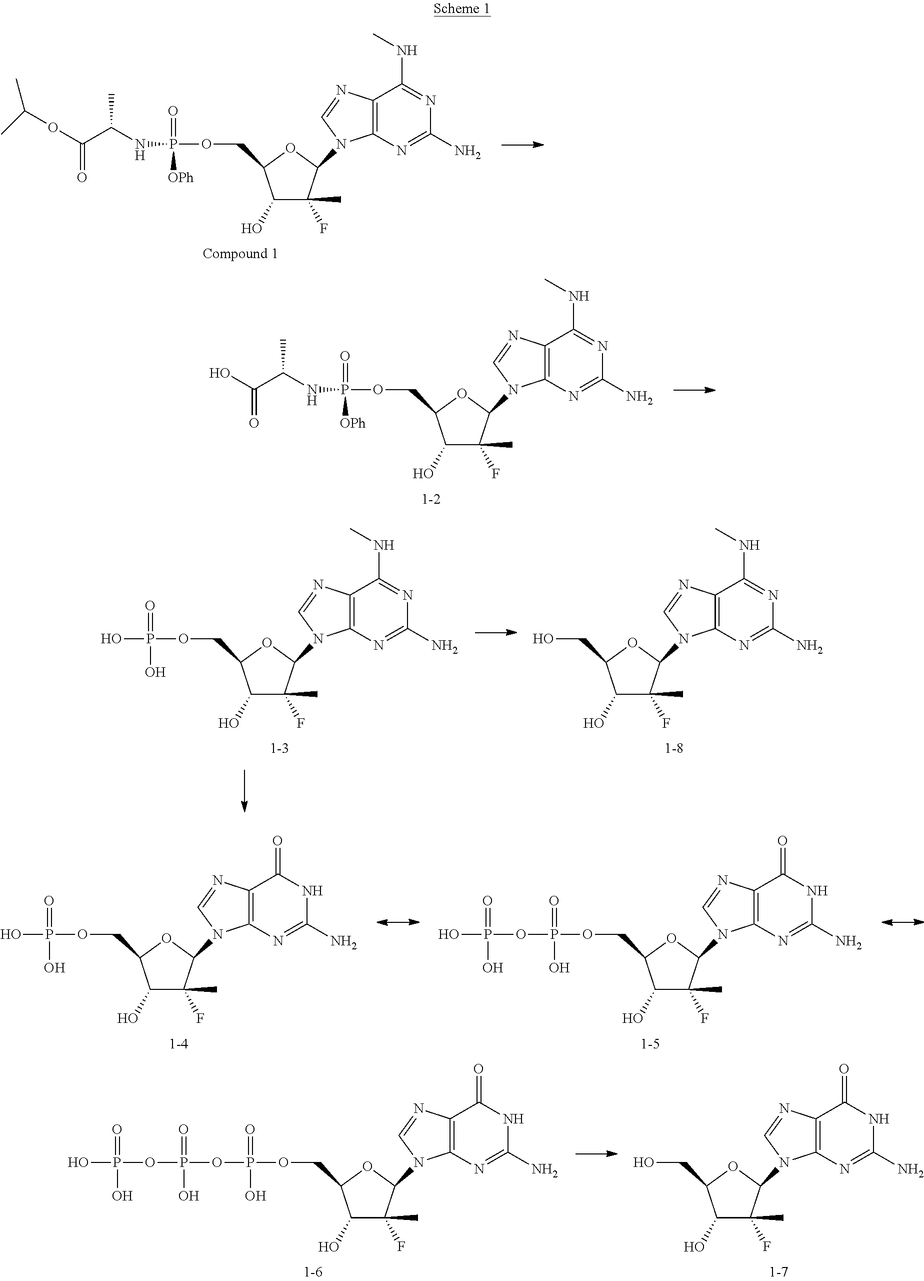

Atea Pharmaceuticals, Inc. has disclosed β-D-2′-deoxy-2′-a-fluoro-2′-β-C-substituted-2-modified-N⁶-(mono- and di-methyl) purine nucleotides for the treatment of HCV in U.S. Pat. Nos. 9,828,410; 10,000,523; 10,005,811; 10,239,911, 10,815,266; 10,870,672; 10,870,673; 10,870,885; 10,519,186; 10,906,928; 10,894,804; and PCT Application Nos. WO 2016/144918; WO 2018/048937; WO 2018/013937; and WO 2018/144640. Atea has also disclosed β-D-2′-deoxy-2′-substituted-4′-substituted-2-N⁶-substituted-6-aminopurine nucleotides for the treatment of paramyxovirus and orthomyxovirus infections in U.S. Pat. No. 10,202,412 and PCT Application No. WO 2018/009623.

### Ruzasvir

Ruzasvir is disclosed in WO 2014/110705 and U.S. Pat. No. 9,555,038 assigned to Merck. In one embodiment, ruzasvir is administered as the pharmaceutically acceptable salt thereof. In one embodiment a solid form of ruzasvir is used. In one embodiment the solid form of ruzasvir is a crystalline solid.

The synthesis of ruzasvir (dimethyl N,N′-([(6S)-6-(2-Cyclopropyl-1,3-thiazol-5-yl)-1-fluoro-6H-indolo[1,2-c][1,3]benzoxazine-3,10-diyl]bis{1H-imidazole-5,2-diyl-(2S)-pyrrolidine-2,1-diyl[(2S)-3-methyl-1-oxobutane-1,2-diyl]})dicarbamate) is known in the art. Non-limiting examples of synthetic methods that can be used to synthesize ruzasvir include those shown in Example 5 and reported in WO 2016/196932 assigned to Merck.

### DEFINITIONS

The term “D-configuration” as used in the context of the present invention refers to the principle configuration which mimics the natural configuration of sugar moieties as opposed to the unnatural occurring nucleosides or “L” configuration. The term “0” or “3 anomer” is used with reference to nucleoside analogs in which the nucleoside base is configured (disposed) above the plane of the furanose moiety in the nucleoside analog.

The term “about”, as used herein, refers to a range that includes up to 10% less than and up to 10% greater than the stated value. For example, “about 100 milligrams” includes all values from 90 milligrams to 110 milligrams as if each value were recited individually.

The terms “coadminister” and “coadministration” or combination therapy are used to describe the administration of Compound 1 or a pharmaceutically acceptable salt thereof according to the present invention in combination with ruzasvir or a pharmaceutically acceptable salt thereof. In certain embodiments, Compound 1 or a pharmaceutically acceptable salt thereof, for example bemnifosbuvir, and ruzasvir or a pharmaceutically acceptable salt thereof are administered with at least one other active agent, for example where appropriate at least one additional anti-HCV agent. The timing of the coadministration with the additional agent is best determined by the medical specialist treating the patient. It is sometimes preferred that the agents be administered at the same time or at least in a manner that allows for an overlapping pharmacologic effect of the two drugs in the treated patient. Alternatively, the drugs selected for combination therapy may be administered at different times to the patient. Of course, when more than one viral or other infection or other condition is present, the present compounds may be combined with other agents to treat that other infection or condition as required.

The term “host”, as used herein refers to a mammal in which an HCV virus can replicate, such as a human or chimpanzee. In typical embodiments, the host is a human.

A “pharmaceutically acceptable salt” is a derivative of the disclosed compound in which the parent compound is modified to an inorganic or organic, acid or base addition salt thereof without undue toxicity. The salts of the present compounds can be synthesized from the parent compound with a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds may optionally be provided in the form of a solvate.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from inorganic or organic acids that are not unduly toxic. For example, conventional acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The compound can be delivered in any molar ratio that delivers the desired result. For example, the compound can be provided with less than a molar equivalent of a counter ion, such as in the form of a hemi-sulfate salt. Alternatively, the compound can be provided with more than molar equivalent of counter ion, such as in the form of a di-sulfate salt. Non-limiting examples of molar ratios of the compound to the counter ion include 1:0.25, 1:0.5, 1:1, and 1:2.

### Isotopic Substitution

The present invention includes combinations of bemnifosbuvir, and ruzasvir or a pharmaceutically acceptable salt thereof wherein one or both of the compounds has desired isotopic substitutions of atoms at amounts above the natural abundance of the isotope, i.e., enriched. Isotopes are atoms having the same atomic number but different mass numbers, i.e., the same number of protons but a different number of neutrons. By way of general example and without limitation, isotopes of hydrogen, for example, deuterium (²H) and tritium (³H) may be used anywhere in described structures. Alternatively, or in addition, isotopes of carbon, e.g., ¹³C and ¹⁴C, may be used. A preferred isotopic substitution is deuterium for hydrogen at one or more locations on the molecule to improve the performance of the drug. The deuterium can be bound in a location of bond breakage during metabolism (an α-deuterium kinetic isotope effect) or next to or near the site of bond breakage (a β-deuterium kinetic isotope effect). Achillion Pharmaceuticals, Inc. (WO/2014/169278 and WO/2014/169280) describes deuteration of nucleotides to improve their pharmacokinetic or pharmacodynamic, including at the 5-position of the molecule.

Substitution with isotopes such as deuterium can afford certain therapeutic advantages resulting from greater metabolic stability, such as, for example, increased in vivo half-life or reduced dosage requirements. Substitution of deuterium for hydrogen at a site of metabolic break-down can reduce the rate of or eliminate the metabolism at that bond. At any position of the compound that a hydrogen atom may be present, the hydrogen atom can be any isotope of hydrogen, including protium (¹H), deuterium (2H) and tritium (3H). Thus, reference herein to a compound encompasses all potential isotopic forms unless the context clearly dictates otherwise.

The term “isotopically-labeled” analog refers to an analog that is a “deuterated analog”, a “¹³C-labeled analog,” or a “deuterated/¹³C-labeled analog.” The term “deuterated analog” means a compound described herein, whereby an H-isotope, i.e., hydrogen/protium (¹H), is substituted by a H-isotope, i.e., deuterium (²H). Deuterium substitution can be partial or complete. Partial deuterium substitution means that at least one hydrogen is substituted by at least one deuterium. In certain embodiments, the isotope is 90, 95 or 99% or more enriched in an isotope at any location of interest. In some embodiments it is deuterium that is 90, 95 or 99% enriched at a desired location. Unless indicated to the contrary, the deuteration is at least 80% at the selected location. Deuteration of the nucleoside can occur at any replaceable hydrogen that provides the desired results.

### Prior Drug Combinations to Treat HCV Infection

A number of fixed-dose drug combinations have been approved for the treatment of HCV. In 2014, the U.S. FDA approved Harvoni® (ledispasvir, a NS5A inhibitor, and sofosbuvir) to treat chronic hepatitis C virus Genotype 1 infection. Harvoni® is the first combination pill approved to treat chronic HCV Genotype 1 infection. It is also the first approved regimen that does not require administration with interferon or ribavirin. In addition, the FDA approved simeprevir (Olysio™) in combination with sofosbuvir (Sovaldi®) as a once-daily, all oral, interferon and ribavirin-free treatment for adults with Genotype 1 HCV infection.

The U.S. FDA also approved AbbVie's VIEKIRA Pak™ in 2014, a multi-pill pack containing dasabuvir (a non-nucleoside NS5B polymerase inhibitor), ombitasvir (a NS5A inhibitor), paritaprevir (a NS3/4A inhibitor), and ritonavir. The VIEKIRA Pak™ can be used with or without ribavirin to treat Genotype 1 HCV infected patients including patients with compensated cirrhosis. VIEKIRA Pak™ does not require interferon co-therapy.

In July 2015, the U.S. FDA approved Technivie™ and Daklinza™ for the treatment of HCV genotype 4 and HCV Genotype 3, respectively. Technivie™ (Ombitasvir/paritaprevir/ritonavir) was approved for use in combination with ribavirin for the treatment of HCV genotype 4 in patients without scarring and cirrhosis and is the first option for HCV-4 infected patients who do not require co-administration with interferon. Daklinza™ was approved for use with Sovaldi® to treat HCV genotype 3 infections. Daklinza™ is the first drug that demonstrated safety and efficacy in treating HCV Genotype 3 without the need for co-administration of interferon or ribavirin. In October 2015, the U.S. FDA warned that HCV treatments Viekira Pak and Technivie can cause serious liver injury primarily in patients with underlying advanced liver disease and required that additional information about safety be added to the label.

In August 2017, Mavyret®, a fixed-dose combination of glecaprevir (an NS3/4A protease inhibitor) and pibrentasvir (an NS5A inhibitor) was approved by the U.S. FDA for the treatment of patients with all major genotypes of HCV (genotypes 1-6). The treatment was also approved for patients without cirrhosis or with mild cirrhosis, those on dialysis, and those with a genotype 1 infection who have been previously treated with a regimen containing an NS5A or an NS3/4A inhibitor. Mavyret® is taken as an 8-week course in non-cirrhotic patients who have not been previously treated. In 2019, the FDA approved an 8-week course in patients who have compensated cirrhosis and have not been previously treated.

Along with Mavyret®, Epclusa® is another therapeutic for HCV treatment. Epclusa was developed by Gilead and is a fixed-dose combination therapy containing sofosbuvir (an NS5B inhibitor) and velpatasvir (an NS5A inhibitor). Epclusa® was approved in 2016 for the treatment of adults with chronic HCV infection of all major genotypes of HCV (genotypes 1-6) and is prescribed for a 12-week course in patients without cirrhosis or with compensated cirrhosis. For patients with decompensated cirrhosis, Epclusa® is approved for use in combination with ribavirin.

United States patents and WO applications that describe nucleoside polymerase inhibitors for the treatment of Flaviviridae, including HCV, include those filed by Idenix Pharmaceuticals (U.S. Pat. Nos. 6,812,219; 6,914,054; 7,105,493; 7,138,376; 7,148,206; 7,157,441; 7,163,929; 7,169,766; 7,192,936; 7,365,057; 7,384,924; 7,456,155; 7,547,704; 7,582,618; 7,608,597; 7,608,600; 7,625,875; 7,635,689; 7,662,798; 7,824,851; 7,902,202; 7,932,240; 7,951,789; 8,193,372; 8,299,038; 8,343,937; 8,362,068; 8,507,460; 8,637,475; 8,674,085; 8,680,071; 8,691,788, 8,742,101, 8,951,985; 9,109,001; 9,243,025; US2016/0002281; US2013/0064794; WO/2015/095305; WO/2015/081133; WO/2015/061683; WO/2013/177219; WO/2013/039920; WO/2014/137930; WO/2014/052638; WO/2012/154321); Merck (U.S. Pat. Nos. 6,777,395; 7,105,499; 7,125,855; 7,202,224; 7,323,449; 7,339,054; 7,534,767; 7,632,821; 7,879,815; 8,071,568; 8,148,349; 8,470,834; 8,481,712; 8,541,434; 8,697,694; 8,715,638, 9,061,041; 9,156,872 and WO/2013/009737); Emory University (U.S. Pat. Nos. 6,348,587; 6,911,424; 7,307,065; 7,495,006; 7,662,938; 7,772,208; 8,114,994; 8,168,583; 8,609,627; US 2014/0212382; and WO2014/1244430); Gilead Sciences/Pharmasset Inc. (7,842,672; 7,973,013; 8,008,264; 8,012,941; 8,012,942; 8,318,682; 8,324,179; 8,415,308; 8,455,451; 8,563,530; 8,841,275; 8,853,171; 8,871,785; 8,877,733; 8,889,159; 8,906,880; 8,912,321; 8,957,045; 8,957,046; 9,045,520; 9,085,573; 9,090,642; and 9,139,604) and (U.S. Pat. Nos. 6,908,924; 6,949,522; 7,094,770; 7,211,570; 7,429,572; 7,601,820; 7,638,502; 7,718,790; 7,772,208; RE42,015; 7,919,247; 7,964,580; 8,093,380; 8,114,997; 8,173,621; 8,334,270; 8,415,322; 8,481,713; 8,492,539; 8,551,973; 8,580,765; 8,618,076; 8,629,263; 8,633,309; 8,642,756; 8,716,262; 8,716,263; 8,735,345; 8,735,372; 8,735,569; 8,759,510 and 8,765,710); Hoffman La-Roche (U.S. Pat. No. 6,660,721), Roche (U.S. Pat. Nos. 6,784,166; 7,608,599, 7,608,601 and 8,071,567); Alios BioPharma Inc. (U.S. Pat. Nos. 8,895,723; 8,877,731; 8,871,737, 8,846,896, 8,772,474; 8,980,865; 9,012,427; US 2015/0105341; US 2015/0011497; US 2010/0249068; US2012/0070411; WO 2015/054465; WO 2014/209979; WO 2014/100505; WO 2014/100498; WO 2013/142159; WO 2013/142157; WO 2013/096680; WO 2013/088155; WO 2010/108135), Enanta Pharmaceuticals (U.S. Pat. Nos. 8,575,119; 8,846,638; 9,085,599; WO 2013/044030; WO 2012/125900), Biota (U.S. Pat. Nos. 7,268,119; 7,285,658; 7,713,941; 8,119,607; 8,415,309; 8,501,699 and 8,802,840), Biocryst Pharmaceuticals (U.S. Pat. Nos. 7,388,002; 7,429,571; 7,514,410; 7,560,434; 7,994,139; 8,133,870; 8,163,703; 8,242,085 and 8,440,813), Alla Chem, LLC (U.S. Pat. No. 8,889,701 and WO 2015/053662), Inhibitex (U.S. Pat. No. 8,759,318 and WO/2012/092484), Janssen Products (U.S. Pat. Nos. 8,399,429; 8,431,588, 8,481,510, 8,552,021, 8,933,052; 9,006,29 and 9,012,428) the University of Georgia Foundation (U.S. Pat. Nos. 6,348,587; 7,307,065; 7,662,938; 8,168,583; 8,673,926, 8,816,074; 8,921,384 and 8,946,244), RFS Pharma, LLC (U.S. Pat. Nos. 8,895,531; 8,859,595; 8,815,829; 8,609,627; 7,560,550; US 2014/0066395; US 2014/0235566; US 2010/0279969; WO/2010/091386 and WO 2012/158811) University College Cardiff Consultants Limited (WO/2014/076490, WO 2010/081082; WO/2008/062206), Achillion Pharmaceuticals, Inc. (WO/2014/169278 and WO 2014/169280), Cocrystal Pharma, Inc. (U.S. Pat. No. 9,173,893), Katholieke Universiteit Leuven (WO 2015/158913), Catabasis (WO 2013/090420) and the Regents of the University of Minnesota (WO 2006/004637).

### Pharmaceutical Compositions and Dosage Forms

A principal aspect of the invention is an advantageous fixed-dose combination comprising bemnifosbuvir and ruzasvir or a pharmaceutically acceptable salt thereof and certain excipients. Selection of excipients in a pharmaceutical composition comprising multiple active compounds can be challenging, as an excipient which improves the bioavailability of one active compound may reduce or even eliminate the bioavailability of the second active compound.

In certain embodiments, the solid dosage form is formulated for oral administration. In certain embodiments, the solid oral dosage form is a pill, tablet, capsule, powder, oral pellet, granule, or lozenge form. In certain embodiments, the advantageous solid dosage form of the invention is a tablet for oral administration.

One of ordinary skill in the art will recognize that a therapeutically effective amount will vary with the infection or condition to be treated, its severity, the treatment regimen to be employed, the pharmacokinetic of the agent used, as well as the patient or subject (animal or human) to be treated, and such therapeutic amount can be determined by the attending physician or specialist.

The amount of bemnifosbuvir and ruzasvir or a pharmaceutically acceptable salt thereof included within the advantageous fixed-dose combination according to the present invention is an effective amount to achieve the desired outcome according to the present invention, for example, for treating the HCV infection, reducing the likelihood of a HCV infection or the inhibition, reduction, and/or abolition of HCV or its secondary effects, including disease states, conditions, and/or complications which occur secondary to HCV. Bemnifosbuvir may for example be administered in amounts ranging from about 0.1 mg/kg to about 15 mg/kg per day of the patient, depending upon the pharmacokinetics of the agent in the patient.

In certain embodiments, about 600 mg of bemnifosbuvir is provided in a dosage form. In certain embodiments, about 300 mg of bemnifosbuvir is provided in a dosage form.

In certain embodiments, the fixed-dose combination comprises from about 100 mg to about 800 mg, from about 150 mg to about 650 mg, from about 200 mg to about 400 mg, from about 300 mg to 600 mg, or from about 500 mg to about 650 mg of bemnifosbuvir in a unit dosage form in addition to from about 10 mg to 500 mg, 45 mg to about 360 mg, or from 50 mg to 150 mg about of ruzasvir or an equivalent amount of a pharmaceutically acceptable salt of ruzasvir.

In certain embodiments, the fixed-dose combination comprises from about 250 mg to about 350 mg of bemnifosbuvir and from about 60 to about 120 mg of ruzasvir.

In certain embodiments, the fixed-dose combination comprises at least about 200, 225, 250, 275, 300, 325, 350, or 375 mg of bemnifosbuvir and from about 60 to about 120 mg of ruzasvir.

In other embodiments, the fixed-dose combination as described herein contains up to about 300 mg of bemnifosbuvir and up to about 45, 90, 135 or 180 mg of ruzasvir or an equivalent amount of a pharmaceutically acceptable salt of ruzasvir. In certain embodiments, a pharmaceutical composition as described herein that contains up to about 300 mg of bemnifosbuvir and up to about 45, 90, 135 or 180 mg of ruzasvir or an equivalent amount of a pharmaceutically acceptable salt of ruzasvir is administered once, two, three, four, five, or six times per day.

In an alternative embodiment, the fixed-dose combination as described contains up to about 300 mg of bemnifosbuvir and up to about 45 mg of ruzasvir or an equivalent amount of a pharmaceutically acceptable salt of ruzasvir. In certain embodiments, a pharmaceutical composition as described herein that contains up to about 300 mg of bemnifosbuvir and up to about 45 mg of ruzasvir or an equivalent amount of a pharmaceutically acceptable salt of ruzasvir is administered once, two, three, four, five, or six times per day.

In an alternative embodiment, the fixed-dose combination as described contains up to about 300 mg of bemnifosbuvir and up to about 90 mg of Ruzasvir or an equivalent amount of a pharmaceutically acceptable salt of ruzasvir. In certain embodiments, a fixed-dose combination as described herein that contains up to about 300 mg of bemnifosbuvir and up to about 90 mg of ruzasvir or an equivalent amount of a pharmaceutically acceptable salt of ruzasvir is administered once, two, three, four, five, or six times per day.

In an alternative embodiment, the fixed-dose combination as described contains up to about 300 mg of bemnifosbuvir and up to about 135 mg of ruzasvir or an equivalent amount of a pharmaceutically acceptable salt of ruzasvir. In certain embodiments, a pharmaceutical composition as described herein that contains up to about 300 mg of bemnifosbuvir and up to about 135 mg of ruzasvir or an equivalent amount of a pharmaceutically acceptable salt of ruzasvir is administered once, two, three, four, five, or six times per day.

In one embodiment, the fixed-dose combination as described herein contains up to about 600 mg of bemnifosbuvir and up to about 90, 180, 270 or 360 mg of ruzasvir or an equivalent amount of a pharmaceutically acceptable salt of ruzasvir. In certain embodiments, a pharmaceutical composition as described herein that contains up to about 600 mg of bemnifosbuvir and up to about 90, 180, 270 or 360 mg of ruzasvir or an equivalent amount of a pharmaceutically acceptable salt of ruzasvir is administered one, two, or three times per day.

In an alternative embodiment, the fixed-dose combination as described contains up to about 600 mg of bemnifosbuvir and up to about 90 mg of ruzasvir or an equivalent amount of a pharmaceutically acceptable salt of ruzasvir. In certain embodiments, a pharmaceutical composition as described herein that contains up to about 600 mg of bemnifosbuvir and up to about 90 mg of ruzasvir or an equivalent amount of a pharmaceutically acceptable salt of ruzasvir is administered one, two, or three times per day.

In an alternative embodiment, the fixed-dose combination as described contains up to about 600 mg of bemnifosbuvir and up to about 180 mg of ruzasvir or an equivalent amount of a pharmaceutically acceptable salt of ruzasvir. In certain embodiments, a pharmaceutical composition as described herein that contains up to about 600 mg of bemnifosbuvir and up to about 180 mg of ruzasvir or an equivalent amount of a pharmaceutically acceptable salt of ruzasvir is administered one, two, or three times per day.

In an alternative embodiment, the fixed-dose combination as described contains up to about 600 mg of bemnifosbuvir and up to about 270 mg of ruzasvir or an equivalent amount of a pharmaceutically acceptable salt of ruzasvir. In certain embodiments, a pharmaceutical composition as described herein that contains up to about 600 mg of bemnifosbuvir and up to about 270 mg of ruzasvir or an equivalent amount of a pharmaceutically acceptable salt of ruzasvir is administered one, two, or three times per day.

In certain embodiments, the fixed-dose combination is administered at least once a day for up to 24 weeks. In certain embodiments, the fixed-dose combination is administered at least once a day for up to 12 weeks. In certain embodiments, the fixed-dose combination is administered at least once a day for up to 10 weeks. In certain embodiments, the fixed-dose combination is administered at least once a day for up to 8 weeks. In certain embodiments, the fixed-dose combination is administered at least once a day for up to 6 weeks. In certain embodiments, the fixed-dose combination is administered at least once a day for up to 4 weeks. In certain embodiments, the fixed-dose combination is administered at least once a day for at least 4 weeks.

In certain embodiments, the fixed-dose combination is administered at least once a day for at least 8 weeks. In certain embodiments, the fixed-dose combination is administered at least once a day for at least 10 weeks. In certain embodiments, the fixed-dose combination is administered at least once a day for at least 12 weeks. In certain embodiments, the fixed-dose combination is administered at least once a day for at least 24 weeks. In certain embodiments, the fixed-dose combination is administered at least every other day for up to 24 weeks, 12 weeks, up to 10 weeks, up to 8 weeks, up to 6 weeks, or up to 4 weeks. In certain embodiments, the fixed-dose combination is administered at least every other day for at least 4 weeks, at least 6 weeks, at least 8 weeks, at least 10 weeks, at least 12 weeks, or at least 24 weeks.

In certain embodiments, the fixed-dose combination is administered at least once a day for 8 weeks to a patient infected with HCV. In certain embodiments, the fixed-dose combination is administered at least once a day for 12 weeks to a patient who is infected with HCV and has developed cirrhosis. In certain embodiments, the fixed-dose combination is administered at least once a day for 6 weeks to a patient who is infected with HCV and has not developed cirrhosis.

For purposes of the present invention, a prophylactically or preventive effective amount of the compositions according to the present invention falls within the same concentration range as set forth above for therapeutically effective amount and is usually the same as a therapeutically effective amount.

To prepare the pharmaceutical compositions according to the present invention, a therapeutically effective amount of bemnifosbuvir and ruzasvir or pharmaceutically acceptable salts thereof may be intimately admixed with specific pharmaceutically acceptable carriers as described herein according to conventional pharmaceutical compounding techniques to produce a dose.

### Solid Dosage Forms

A principal aspect of the invention is a fixed dosage form comprising bemnifosbuvir, a secondary formulation comprising ruzasvir, and certain excipients as described in more detail herein.

In certain embodiments, the fixed-dose combination comprises an intragranular mix and an extragranular mix, wherein the intragranular mix comprises
(i) bemnifosbuvir;
(ii) a secondary formulation comprising ruzasvir; and
(iii) one or more excipients selected from a filler, a disintegrant, a glidant, a lubricant, and an osmogen, as described herein;
and the extragranular mix comprises one or more excipients selected from a disintegrant, a glidant, and a lubricant.

In certain embodiments, the intragranular mix is blended, compressed, and screened to form a solid dispersion. In certain embodiments, the intragranular mix solid dispersion is further blended with the extragranular mix. In certain embodiments, the extragranular mix comprises one or more excipients selected from a disintegrant, a glidant, and a lubricant. In certain embodiments, the extragranular mix comprises a disintegrant, a glidant, and a lubricant.

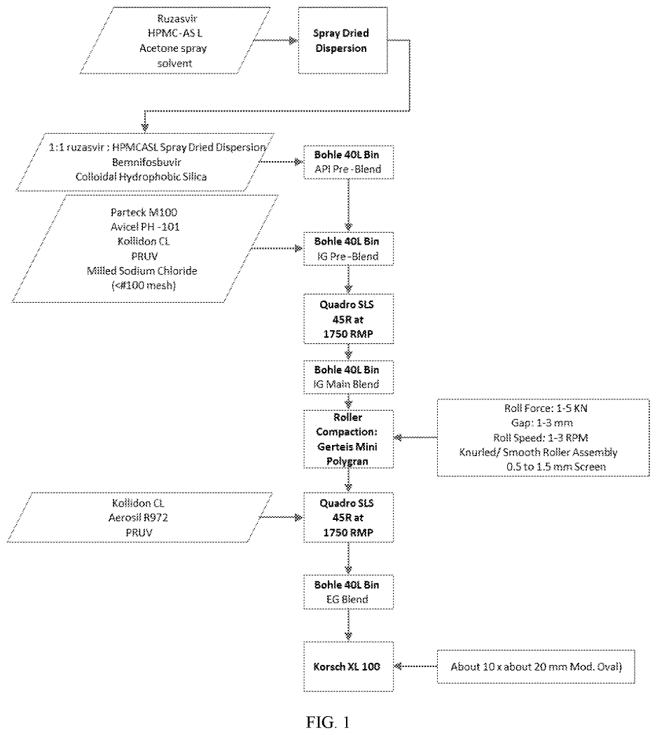

In certain embodiments, the blend comprising the intragranular mix and the extragranular mix is formed into the solid dosage form. In certain embodiments, the blend comprising the intragranular mix and the extragranular mix is compressed into tablets. In certain embodiments, the blend comprising the intragranular mix and the extragranular mix is compressed into oral pellets. In certain embodiments, the blend comprising the intragranular mix and the extragranular mix is filled into capsules. In certain embodiments, the blend comprising the intragranular mix and the extragranular mix is compressed and screened into granules. An exemplary process for preparing solid dosage forms for oral delivery can be found in FIG. 1.

In certain embodiments, bemnifosbuvir is provided in a crystalline form. In certain embodiments, bemnifosbuvir is provided in a crystalline form characterized by three or more XRPD peaks selected from 5.2±0.2°, 7.3±0.2°, 8.9±0.2°, 13.6±0.2°, 17.0±0.2°, 19.9±0.2°, and 21.8±0.2° 2theta. In certain embodiments, bemnifosbuvir is provided in a crystalline form characterized by four or more XRPD peaks selected from 5.2±0.2°, 7.3±0.2°, 8.9±0.2°, 13.6±0.2°, 17.0±0.2°, 19.9±0.2°, and 21.8±0.2° 2theta. In certain embodiments, bemnifosbuvir is provided in a crystalline form characterized by five or more XRPD peaks selected from 5.2±0.2°, 7.3±0.2°, 8.9±0.2°, 13.6±0.2°, 17.0±0.2°, 19.9±0.2°, and 21.8±0.2° 2theta. In certain embodiments, bemnifosbuvir is provided in a crystalline form characterized by six or more XRPD peaks selected from 5.2±0.2°, 7.3±0.2°, 8.9±0.2°, 13.6±0.2°, 17.0±0.2°, 19.9±0.2°, and 21.8±0.2° 2theta.

In certain embodiments, ruzasvir is provided as an amorphous solid. In certain embodiments, ruzasvir is provided as a spray-dried solid. In certain embodiments, ruzasvir is provided as a spray-dried dispersion in a pharmaceutically acceptable carrier. In certain embodiments, the spray-dried dispersion comprising ruzasvir includes hydroxypropylmethyl cellulose acetate succinate (HPMCAS). In certain embodiments, the spray-dried dispersion comprising ruzasvir includes HPMCAS-L. HPMCAS is produced in at least three grades, L, M, and H. The different grades correspond to the degree of acetylation and succinoylation on the sugar polymer backbone. In certain embodiments, HPMCAS-L comprises 5-9% acetyl content. In certain embodiments, HPMCAS-L comprises 14-18% succinoyl content. In certain embodiments, HPMCAS-L comprises 20-24% methoxyl content. In certain embodiments, HPMCAS-L comprises 5-9% hydroxypropoxy content. In certain embodiments, the spray-dried dispersion comprising ruzasvir includes HPMCAS-M. In certain embodiments, the spray-dried dispersion comprising ruzasvir includes HPMCAS-H.

### Embodiments of the Solid Dosage Form

In certain embodiments, the fixed-dose combination comprises an intragranular mix and an extragranular mix, wherein the intragranular mix comprises
(i) bemnifosbuvir, in a crystalline form;
(ii) a spray-dried dispersion of ruzasvir and HPMCAS-L; and
(iii) a filler, a disintegrant, a glidant, a lubricant, and an osmogen, as described herein;
and the extragranular mix comprises one or more excipients selected from a disintegrant, a glidant, and a lubricant.

In certain embodiments, the fixed-dose combination drug product comprises:
a) bemnifosbuvir optionally in a morphic form;
b) a spray-dried dispersion of ruzasvir, or a pharmaceutically acceptable salt thereof, in hydroxyalkylcellulose;
c) a mixture of
   i) a cellulosic polymer;
   ii) a sugar alcohol;
   iii) a disintegrant;
   iv) a glidant;
   v) a lubricant; and
   vi) an osmogen;
   vii) wherein the components are combined to create an intragranular mix;
d) combining the intragranular mix of c) with an extragranular material to form a solid material that holds the intragranular mix together; and
e) optionally coating with a tablet coating material to create the solid dosage form.

In certain embodiments, the fixed-dose combination comprises an intragranular mix and an extragranular mix, wherein the intragranular mix comprises
(i) bemnifosbuvir, in a crystalline form;
(ii) a spray-dried dispersion of ruzasvir and HPMCAS-L; and
(iii) one or more of microcrystalline cellulose, mannitol, crospovidone, silicon dioxide, sodium stearyl fumarate, and sodium chloride;
and the extragranular mix comprises one or more excipients selected from a disintegrant, a glidant, and a lubricant.

In certain embodiments, the fixed-dose combination comprises an intragranular mix and an extragranular mix, wherein the intragranular mix comprises
(i) bemnifosbuvir, in a crystalline form;
(ii) a spray-dried dispersion of ruzasvir and HPMCAS-L; and
(iii) microcrystalline cellulose, mannitol, crospovidone, silicon dioxide, sodium stearyl fumarate, and sodium chloride;
and the extragranular mix comprises one or more excipients selected from a disintegrant, a glidant, and a lubricant.

In certain embodiments, the fixed-dose combination comprises an intragranular mix and an extragranular mix, wherein the intragranular mix comprises
(i) bemnifosbuvir, in a crystalline form;
(ii) a spray-dried dispersion comprising from about 40% to about 60% ruzasvir and HPMCAS-L; and
(iii) microcrystalline cellulose, mannitol, crospovidone, silicon dioxide, sodium stearyl fumarate, and sodium chloride;
and the extragranular mix comprises one or more excipients selected from a disintegrant, a glidant, and a lubricant.

In certain embodiments, the fixed-dose combination comprises an intragranular mix and an extragranular mix, wherein the intragranular mix comprises
(i) bemnifosbuvir, in a crystalline form;
(ii) a spray-dried dispersion comprising about 50% ruzasvir and at least about 40% HPMCAS-L; and
(iii) one or more of microcrystalline cellulose, mannitol, crospovidone, silicon dioxide, sodium stearyl fumarate, and sodium chloride;
and the extragranular mix comprises one or more excipients selected from a disintegrant, a glidant, and a lubricant.

In certain embodiments, the fixed-dose combination comprises an intragranular mix and an extragranular mix, wherein the intragranular mix comprises
(i) bemnifosbuvir, in a crystalline form;
(ii) a spray-dried dispersion comprising about 50% ruzasvir and at least about 40% HPMCAS-L; and
(iii) microcrystalline cellulose, mannitol, crospovidone, silicon dioxide, sodium stearyl fumarate, and sodium chloride;
and the extragranular mix comprises one or more excipients selected from a disintegrant, a glidant, and a lubricant.

In certain embodiments, the fixed-dose combination comprises an intragranular mix and an extragranular mix, wherein the intragranular mix comprises
(i) bemnifosbuvir, in a crystalline form;
(ii) a spray-dried dispersion comprising about 50% ruzasvir and about 50% HPMCAS-L; and
(iii) microcrystalline cellulose, mannitol, crospovidone, silicon dioxide, sodium stearyl fumarate, and sodium chloride;
and the extragranular mix comprises one or more excipients selected from a disintegrant, a glidant, and a lubricant.

In certain embodiments, the fixed-dose combination comprises an intragranular mix and an extragranular mix, wherein the intragranular mix comprises
(i) from about 250 mg to about 350 mg of bemnifosbuvir in a crystalline form; (ii) a spray-dried dispersion comprising about 50% ruzasvir and about 50% HPMCAS-L; and
(iii) one or more of microcrystalline cellulose, mannitol, crospovidone, silicon dioxide, sodium stearyl fumarate, and sodium chloride;
and the extragranular mix comprises one or more excipients selected from a disintegrant, a glidant, and a lubricant.

In certain embodiments, the fixed-dose combination comprises an intragranular mix and an extragranular mix, wherein the intragranular mix comprises
(i) from about 250 mg to about 350 mg of bemnifosbuvir in a crystalline form;
(ii) a spray-dried dispersion comprising about 50% ruzasvir and about 50% HPMCAS-L; and
(iii) one or more of microcrystalline cellulose, mannitol, crospovidone, silicon dioxide, sodium stearyl fumarate, and sodium chloride;
and the extragranular mix comprises one or more excipients selected from a disintegrant, a glidant, and a lubricant.

In certain embodiments, the fixed-dose combination comprises an intragranular mix and an extragranular mix, wherein the intragranular mix comprises
(i) from about 250 mg to about 350 mg of bemnifosbuvir in a crystalline form;
(ii) from about 150 mg to about 250 mg of a spray-dried dispersion comprising about 50% ruzasvir and about 50% HPMCAS-L; and
(iii) one or more of microcrystalline cellulose, mannitol, crospovidone, silicon dioxide, sodium stearyl fumarate, and sodium chloride;
and the extragranular mix comprises one or more excipients selected from a disintegrant, a glidant, and a lubricant.

In certain embodiments, the fixed-dose combination comprises an intragranular mix and an extragranular mix, wherein the intragranular mix comprises
(i) from about 250 mg to about 350 mg of bemnifosbuvir in a crystalline form;
(ii) from about 150 mg to about 250 mg of a spray-dried dispersion comprising about 50% ruzasvir and about 50% HPMCAS-L; and
(iii) microcrystalline cellulose, mannitol, crospovidone, silicon dioxide, sodium stearyl fumarate, and sodium chloride;
and the extragranular mix comprises one or more excipients selected from a disintegrant, a glidant, and a lubricant.

In certain embodiments, the fixed-dose combination comprises an intragranular mix and an extragranular mix, wherein the intragranular mix comprises:

| Intragranular Mix Component | Amount (milligrams) |
|---|---|
| Bemnifosbuvir | From about 250 mg to about 350 mg |
| Spray-dried dispersion | From about 150 mg to about 250 mg |
| comprising about 50% ruzasvir | |
| and about 50% HPMCAS-L | |
| Microcrystalline cellulose | From about 300 mg to about 450 mg |
| Mannitol | From about 125 mg to about 250 mg |
| Crospovidone | From about 90 mg to about 140 mg |
| Silicon dioxide | From about 5 mg to about 35 mg |
| Sodium stearyl fumarate | From about 5 mg to about 35 mg |
| Sodium chloride | From about 50 mg to about 150 mg |

an the extragranular mix comprises one or more excipients selected from a disintegrant, a glidant, and a lubricant.

In certain embodiments, the fixed-dose combination comprises an intragranular mix and an extragranular mix, wherein the intragranular mix comprises:

| Intragranular Mix Component | Amount (wt %) |
|---|---|
| Bemnifosbuvir, in a | From about 15% to about 30%, |
| crystalline form | typically from about 20% to |
| | about 25% |
| Spray-dried dispersion | From about 5% to about 25%, |
| comprising about 50% ruzasvir | typically from about 12% to |
| and about 50% HPMCAS-L | about 27% |
| Microcrystalline cellulose | From about 15% to about 30%, |
| | typically from about 22% to |
| | about 29% |
| Sugar alcohol, ,such as | From about 7% to about 20%, |
| Mannitol | typically from about 10% to |
| | about 15% |
| Degradant such as | From about 5% to about 15%, |
| Crospovidone | typically from about 8% to |
| | about 12% |
| Glidant such as | From about 0.5% to about 5%, |
| Silicon dioxide | typically from about 1% to 2% |
| Lubricant, such as Sodium | From about 0.5% to about 5%, |
| stearyl fumarate | typically from about 1% to 2% |
| Osmogen, such as Sodium | From about 3% to about 12%, |
| chloride | typically from about 5% to |
| | about 10% |
| Total | 100%, in proportions selected |
| | from the ranges such that the |
| | total is 100% |

and
the extragranular mix comprises one or more excipients selected from a disintegrant, a glidant, and a lubricant.

In certain embodiments, the fixed-dose combination comprises an intragranular mix and an extragranular mix, wherein the intragranular mix comprises:

| Intragranular Mix Component | Amount (milligrams) |
|---|---|
| Bemnifosbuvir, in a | From about 250 mg to about 350 mg |
| crystalline form | |
| Spray-dried dispersion | From about 150 mg to about 250 mg |
| comprising about 50% ruzasvir | |
| and about 50% HPMCAS-L | |
| Microcrystalline cellulose | From about 300 mg to about 450 mg |
| Mannitol | From about 125 mg to about 250 mg |
| Crospovidone | From about 90 mg to about 140 mg |
| Silicon dioxide | From about 5 mg to about 35 mg |
| Sodium stearyl fumarate | From about 5 mg to about 35 mg |
| Sodium chloride | From about 50 mg to about 150 mg |

and the extragranular mix comprises:

| | |
|---|---|
| Crospovidone | From about 15 mg to about 45 mg |
| Silicon dioxide | From about 2 mg to about 20 mg |
| Sodium stearyl fumarate | From about 2 mg to about 20 mg |

In certain embodiments, the fixed-dose combination comprises an intragranular mix and an extragranular mix, wherein the intragranular mix comprises:

| Intragranular Mix Component | Amount (wt %) |
|---|---|
| Bemnifosbuvir, in a | From about 15% to about 30%, |
| crystalline form | typically from about 20% to |
| | about 25% |
| Spray-dried dispersion | From about 5% to about 25%, |
| comprising about 50% ruzasvir | typically from about 12% to |
| and about 50% HPMCAS-L | about 27% |
| Microcrystalline cellulose | From about 15% to about 30%, |
| | typically from about 22% to |
| | about 29% |
| Mannitol | From about 7% to about 20%, |
| | typically from about 10% to |
| | about 15% |
| Crospovidone | From about 5% to about 15%, |
| | typically from about 8% to |
| | about 12% |
| Silicon dioxide | From about 0.5% to about 5%, |
| | typically from about 1% to 2% |
| Sodium stearyl fumarate | From about 0.5% to about 5%, |
| | typically from about 1% to 2% |
| Sodium chloride | From about 3% to about 12%, |
| | typically from about 5% to |
| | about 10% |
| Total Intragranular | From about 90% to about 99% |

and the extragranular mix comprises:

| | |
|---|---|
| Crospovidone | From about 0.5% to about 5%, |
| | typically from about 1% to 3% |
| Silicon dioxide | From about 0.05% to about 2%, |
| | typically from about 0.1% to 1% |
| Sodium stearyl fumarate | From about 0.05% to about 2%, |
| | typically from about 0.1% to 1% |
| Total Extragranular | From about 1% to about 10% |

Wherein the intragranular and extragranular excipients are in proportions selected from the ranges such that the total of Intragranular components and Extragranular components totals 100%.

In certain embodiments, the fixed-dose combination comprises an intragranular mix and an extragranular mix, wherein the intragranular mix comprises:

| Intragranular Mix Component | Amount (milligrams) |
|---|---|
| Bemnifosbuvir, in a | From about 290 mg to about 310 mg |
| crystalline form | |
| Spray-dried dispersion | From about 175 mg to about 185 mg |
| comprising about 50% ruzasvir | |
| and about 50% HPMCAS-L | |
| Microcrystalline cellulose | From about 340 mg to about 350 mg |
| Mannitol | From about 170 mg to about 180 mg |
| Crospovidone | From about 87 mg to about 95 mg |
| Silicon dioxide | From about 10 mg to about 15 mg |
| Sodium stearyl fumarate | From about 10 mg to about 15 mg |
| Sodium chloride | From about 85 mg to about 95 mg |

and the extragranular mix comprises:

| | |
|---|---|
| Crospovidone | From about 20 mg to about 30 mg |
| Silicon dioxide | From about 5 mg to about 7 mg |
| Sodium stearyl fumarate | From about 5 mg to about 7 mg |

In certain embodiments, the fixed-dose combination comprises an intragranular mix and an extragranular mix, wherein the intragranular mix comprises:

| Intragranular Mix Component | Amount (milligrams) |
|---|---|
| Bemnifosbuvir, in a crystalline form | About 300 mg |
| Spray-dried dispersion comprising about | About 180 mg |
| 50% ruzasvir and about 50% HPMCAS-L | |
| Microcrystalline cellulose | About 350 mg |
| Mannitol | About 175 mg |
| Crospovidone | About 90 mg |
| Silicon dioxide | About 12 mg |
| Sodium stearyl fumarate | About 12 mg |
| Sodium chloride | About 90 mg |

and the extragranular mix comprises:

| | |
|---|---|
| Crospovidone | About 25 mg |
| Silicon dioxide | About 6 mg |
| Sodium stearyl fumarate | About 6 mg |

### Alternative Embodiments of the Fixed-Dose Combination

In certain embodiments, the fixed-dose combination comprises:

| Intragranular Mix Component | Amount (milligrams) |
|---|---|
| Bemnifosbuvir, in a crystalline | From about 250 mg to about 350 mg |
| form | |
| Amorphous ruzasvir | From about 45 mg to about 135 mg |
| Microcrystalline cellulose | From about 200 mg to about 300 mg |
| Mannitol | From about 100 mg to about 150 mg |
| Crospovidone | From about 60 mg to about 120 mg |
| Colloidal Silicon dioxide | From about 5 mg to about 35 mg |
| Sodium stearyl fumarate | From about 5 mg to about 35 mg |
| Sodium lauryl sulfate | From about 60 mg to about 120 mg |

In certain embodiments, the fixed-dose combination comprises:

| Intragranular Mix Component | Amount (wt %) |
|---|---|
| Bemnifosbuvir, in a crystalline | From about 20% to about 40%, |
| form | typically from about 25% to about 35% |
| Spray-dried dispersion | From about 5% to about 20%, |
| comprising about 50% ruzasvir | typically from about 7% to about 12% |
| and about 50% HPMCAS-L | |
| Microcrystalline cellulose | From about 15% to about 30%, |
| | typically from about 22% to about 29% |
| Mannitol | From about 7% to about 20%, |
| | typically from about 10% to about 15% |
| Crospovidone | From about 5% to about 15%, |
| | typically from about 8% to about 12% |
| Silicon dioxide | From about 0.5% to about 5%, |
| | typically from about 1% to 2% |
| Sodium stearyl fumarate | From about 0.5% to about 5%, |
| | typically from about 1% to 2% |
| Sodium lauryl sulfate | From about 5% to about 15%, |
| | typically from about 8% to about 12% |
| Total | 100%, in proportions selected from the |
| | ranges such that the total is 100% |

In certain embodiments, the fixed-dose combination comprises:

| Intragranular Mix Component | Amount (milligrams) |
|---|---|
| Bemnifosbuvir, in a crystalline | From about 250 mg to about 350 mg |
| form | |
| Amorphous ruzasvir | From about 45 mg to about 135 mg |
| Microcrystalline cellulose | From about 275 mg to about 350 mg |
| Mannitol | From about 130 mg to about 190 mg |
| Crospovidone | From about 80 mg to about 140 mg |
| Colloidal Silicon dioxide | From about 5 mg to about 35 mg |
| Sodium stearyl fumarate | From about 5 mg to about 35 mg |
| Sodium chloride | From about 25 mg to about 75 mg |
| Sodium lauryl sulfate | From about 90 mg to about 140 mg |

In certain embodiments, the fixed-dose combination comprises:

| Intragranular Mix Component | Amount (wt %) |
|---|---|
| Bemnifosbuvir, in a crystalline | From about 20% to about 40%, |
| form | typically from about 20% to about 30% |
| Amorphous ruzasvir | From about 3% to about 15%, |
| | typically from about 5% to about 10% |
| Microcrystalline cellulose | From about 15% to about 30%, |
| | typically from about 22% to about 29% |
| Mannitol | From about 7% to about 20%, |
| | typically from about 10% to about 15% |
| Crospovidone | From about 5% to about 15%, |
| | typically from about 8% to about 12% |
| Colloidal silicon dioxide | From about 0.5% to about 5%, |
| | typically from about 1% to 2% |
| Sodium stearyl fumarate | From about 0.5% to about 5%, |
| | typically from about 1% to 3% |
| Sodium chloride | From about 2% to about 10%, |
| | typically from about 3% to about 8% |
| Sodium lauryl sulfate | From about 5% to about 15%, |
| | typically from about 8% to about 12% |
| Total | 100%, in proportions selected from the |
| | ranges such that the total is 100% |

In certain embodiments, the fixed-dose combination comprises:

| Intragranular Mix Component | Amount (milligrams) |
|---|---|
| Bemnifosbuvir, in a crystalline | From about 250 mg to about 350 mg |
| form | |
| Spray-dried dispersion | From about 135 mg to about 235 mg |
| comprising about 50% ruzasvir | |
| and about 50% HPMCAS-L | |
| Microcrystalline cellulose | From about 275 mg to about 350 mg |
| Mannitol | From about 130 mg to about 190 mg |
| Crospovidone | From about 80 mg to about 140 mg |
| Silicon dioxide | From about 5 mg to about 35 mg |
| Sodium stearyl fumarate | From about 5 mg to about 35 mg |
| Sodium chloride | From about 75 mg to about 125 mg |
| Sodium lauryl sulfate | From about 15 mg to about 40 mg |

In certain embodiments, the fixed-dose combination comprises:

| Intragranular Mix Component | Amount (wt %) |
|---|---|
| Bemnifosbuvir, in a crystalline | From about 20% to about 40%, |
| form | typically from about 20% to |
| | about 30% |
| Spray-dried dispersion comprising | From about 5% to about 25%, |
| about 50% ruzasvir and about | typically from about 10% to |
| 50% HPMCAS-L | about 20% |
| Microcrystalline cellulose | From about 15% to about 30%, |
| | typically from about 22% to |
| | about 29% |
| Mannitol | From about 7% to about 20%, |
| | typically from about 10% to |
| | about 15% |
| Crospovidone | From about 5% to about 15%, |
| | typically from about 8% to |
| | about 12% |
| Silicon dioxide | From about 0.5% to about 5%, |
| | typically from about 1% to 2% |
| Sodium stearyl fumarate | From about 0.5% to about 5%, |
| | typically from about 1% to 3% |
| Sodium chloride | From about 2% to about 10%, |
| | typically from about 3% to |
| | about 8% |
| Sodium lauryl sulfate | From about 0.5% to about 5%, |
| | typically from about 1% to 3% |
| Total | 100%, in proportions selected |
| | from the ranges such that the |
| | total is 100% |

In certain embodiments, the fixed-dose combination comprises:

| Intragranular Mix Component | Amount (milligrams) |
|---|---|
| Bemnifosbuvir, in a crystalline | From about 250 mg to about 350 mg |
| form | |
| Spray-dried dispersion | From about 350 mg to about 450 mg |
| comprising ruzasvir and | |
| HPMC-E3 | |
| Microcrystalline cellulose | From about 275 mg to about 350 mg |
| Mannitol | From about 130 mg to about 190 mg |
| Croscarmellose sodium | From about 120 mg to about 180 mg |
| Colloidal silicon dioxide | From about 5 mg to about 15 mg |
| Magnesium stearate | From about 15 mg to about 35 mg |
| Sodium chloride | From about 130 mg to about 180 mg |

In certain embodiments, the fixed-dose combination comprises:

| Intragranular Mix Component | Amount (wt %) |
|---|---|
| Bemnifosbuvir, in a crystalline | From about 12% to about 30%, |
| form | typically from about 15% to about 22% |
| Spray-dried dispersion | From about 15% to about 35%, |
| comprising ruzasvir and | typically from about 20% to about 30% |
| HPMC-E3 | |
| Microcrystalline cellulose | From about 15% to about 30%, |
| | typically from about 18% to about 25% |
| Mannitol | From about 7% to about 20%, |
| | typically from about 8% to about 15% |
| Croscarmellose sodium | From about 5% to about 15%, |
| | typically from about 8% to about 12% |
| Colloidal silicon dioxide | From about 0.05% to about 5%, |
| | typically from about 0.1% to 2% |
| Magnesium stearate | From about 0.05% to about 5%, |
| | typically from about 0.5% to 2% |
| Sodium chloride | From about 5% to about 15%, |
| | typically from about 8% to about 12% |
| Total | 100%, in proportions selected from |
| | the ranges such that the total is 100% |

In certain embodiments, the fixed-dose combination comprises an intragranular mix and an extragranular mix, wherein the intragranular mix comprises:

| Intragranular Mix Component | Amount (milligrams) |
|---|---|
| Bemnifosbuvir, in a crystalline | From about 250 mg to about 350 mg |
| form | |
| Spray-dried dispersion comprising | From about 150 mg to about 250 mg |
| about 50% ruzasvir and about | |
| 50% HPMCAS-L | |
| Granulation comprising | From about 500 mg to about 650 mg |
| microcrystalline cellulose, | |
| mannitol, and Vitamin E TPGS | |
| Mannitol | From about 90 mg to about 125 mg |
| Croscarmellose sodium | From about 70 mg to about 110 mg |
| Colloidal silicon dioxide | From about 5 mg to about 35 mg |
| Sodium stearyl fumarate | From about 5 mg to about 35 mg |
| Sodium chloride | From about 50 mg to about 150 mg |

and the extragranular mix comprises:

| | |
|---|---|
| Croscarmellose sodium | From about 30 mg to about 75 mg |
| Colloidal silicon dioxide | From about 2 mg to about 15 mg |
| Sodium stearyl fumarate | From about 2 mg to about 15 mg |

In certain embodiments, the fixed-dose combination comprises an intragranular mix and an extragranular mix, wherein the intragranular mix comprises:

| Intragranular Mix Component | Amount (wt %) |
|---|---|
| Bemnifosbuvir, in a crystalline | From about 15% to about 25%, |
| form | typically from about 18% to |
| | about 22% |
| Spray-dried dispersion comprising | From about 5% to about 20%, |
| about 50% ruzasvir and about | typically from about 8% to |
| 50% HPMCAS-L | about 15% |
| Granulation comprising | From about 30% to about 50%, |
| microcrystalline cellulose, | typically from about 35% to |
| mannitol, and Vitamin E TPGS | about 45% |
| Mannitol | From about 2% to about 10%, |
| | typically from about 5% to |
| | about 9% |
| Croscarmellose sodium | From about 2% to about 10%, |
| | typically from about 5% to |
| | about 9% |
| Colloidal silicon dioxide | From about 0.1% to about 5%, |
| | typically from about 0.5% to 2% |
| Sodium stearyl fumarate | From about 0.1% to about 5%, |
| | typically from about 0.5% to 2% |
| Sodium chloride | From about 3% to about 12%, |
| | typically from about 5% to |
| | about 10% |
| Total Intragranular | From about 90% to about 98% |

and the extragranular mix comprises:

| | |
|---|---|
| Croscarmellose sodium | From about 1% to about 8%, |
| | typically from about 3% to 6% |
| Colloidal silicon dioxide | From about 0.05% to about 2%, |
| | typically from about 0.1% to 1% |
| Sodium stearyl fumarate | From about 0.05% to about 2%, |
| | typically from about 0.1% to 1% |
| Total Extragranular | From about 2% to about 10% |

Wherein the intragranular and extragranular excipients are in proportions selected from the ranges such that the total of Intragranular components and Extragranular components totals 100%.

In certain embodiments, the fixed-dose combination comprises an intragranular mix and an extragranular mix, wherein the intragranular mix comprises:

| Intragranular Mix Component | Amount (milligrams) |
|---|---|
| Bemnifosbuvir, in a crystalline | From about 250 mg to about 350 mg |
| form | |
| Amorphous ruzasvir | From about 60 mg to about 120 mg |
| Granulation comprising | From about 500 mg to about 650 mg |
| microcrystalline cellulose, | |
| mannitol, and Vitamin E TPGS | |
| Mannitol | From about 175 mg to about 230 mg |
| Croscarmellose sodium | From about 70 mg to about 110 mg |
| Colloidal silicon dioxide | From about 5 mg to about 35 mg |
| Sodium stearyl fumarate | From about 5 mg to about 35 mg |
| Sodium chloride | From about 50 mg to about 150 mg |

and the extragranular mix comprises:

| | |
|---|---|
| Croscarmellose sodium | From about 30 mg to about 75 mg |
| Colloidal silicon dioxide | From about 2 mg to about 15 mg |
| Sodium stearyl fumarate | From about 2 mg to about 15 mg |

In certain embodiments, the fixed-dose combination comprises an intragranular mix and an extragranular mix, wherein the intragranular mix comprises:

| Intragranular Mix Component | Amount (wt %) |
|---|---|
| Bemnifosbuvir, in a crystalline | From about 15% to about 25%, |
| form | typically from about 18% to about 22% |
| Amorphous ruzasvir | From about 3% to about 10%, |
| | typically from about 5% to about 8% |
| Granulation comprising | From about 30% to about 50%, |
| microcrystalline cellulose, | typically from about 35% to about 45% |
| mannitol, and Vitamin E TPGS | |
| Mannitol | From about 5% to about 20%, |
| | typically from about 10% to about 15% |
| Croscarmellose sodium | From about 2% to about 10%, |
| | typically from about 5% to about 9% |
| Colloidal silicon dioxide | From about 0.1% to about 5%, |
| | typically from about 0.5% to 2% |
| Sodium stearyl fumarate | From about 0.1% to about 5%, |
| | typically from about 0.5% to 2% |
| Sodium chloride | From about 3% to about 12%, |
| | typically from about 5% to about 10% |
| Total Intragranular | From about 90% to about 98% |

and the extragranular mix comprises:

| | |
|---|---|
| Croscarmellose sodium | From about 1% to about 8%, |
| | typically from about 3% to 6% |
| Colloidal silicon dioxide | From about 0.05% to about 2%, |
| | typically from about 0.1% to 1% |
| Sodium stearyl fumarate | From about 0.05% to about 2%, |
| | typically from about 0.1% to 1% |
| Total Extragranular | From about 2% to about 10% |

Wherein the intragranular and extragranular excipients are in proportions selected from the ranges such that the total of Intragranular components and Extragranular components totals 100%.

In certain embodiments, the fixed-dose combination comprises:

| Intragranular Mix Component | Amount (milligrams) |
|---|---|
| Bemnifosbuvir, in a | From about 250 mg to about 350 mg |
| crystalline form | |
| Amorphous ruzasvir | From about 60 mg to about 120 mg |
| Microcrystalline cellulose | From about 375 mg to about 550 mg |
| Mannitol | From about 190 mg to about 250 mg |
| Crospovidone | From about 120 mg to about 180 mg |
| Silicon dioxide | From about 5 mg to about 35 mg |
| Sodium stearyl fumarate | From about 5 mg to about 20 mg |
| Fumaric acid | From about 110 mg to about 160 mg |

In certain embodiments, the fixed-dose combination comprises:

| Intragranular | |
|---|---|
| Mix Component | Amount (wt %) |
| Bemnifosbuvir, in a | From about 12% to about 30%, |
| crystalline form | typically from about 15% to about 25% |
| Amorphous ruzasvir | From about 2% to about 15%, |
| | typically from about 5% to about 10% |
| Microcrystalline | From about 25% to about 37%, |
| cellulose | typically from about 30% to about 35% |
| Mannitol | From about 10% to about 20%, |
| | typically from about 14% to about 18% |
| Crospovidone | From about 5% to about 15%, |
| | typically from about 8% to about 12% |
| Silicon dioxide | From about 0.05% to about 5%, |
| | typically from about 0.5% to 2% |
| Sodium stearyl | From about 0.05% to about 5%, |
| fumarate | typically from about 0.5% to 2% |
| Fumaric acid | From about 5% to about 15%, |
| | typically from about 8% to about 12% |
| Total | 100%, in proportions selected from the |
| | ranges such that the total is 100%. |

In certain embodiments, the fixed-dose combination described herein is coated. In certain embodiments, the coating comprises hypromellose. In certain embodiments, the coating comprises lactose. In certain embodiments, the coating comprises hydroxypropyl cellulose. In certain embodiments, the coating comprises titanium dioxide. In certain embodiments, the coating is Opadry® white.

In certain embodiments, the coating is applied to the fixed-dose combination at a rate of from about 1% to about 10%, from about 2% to about 5% or from about 3% to about 4%, wherein the weight percent refers to coating the tablet until the specified weight percent is added to the composition.

In certain embodiments, the fixed-dose combination further comprises a tablet coating. In certain embodiments, the tablet coating is a film coating. In certain embodiments the tablet coating is Opadry® II white. In certain embodiments, the tablet is coated with about 0.5, 1, 1.5, 2, 2.5, 3, 3.5, 4, 4.5, 5, 5.5, 6, 6.5, 7, or 7.5 weight percent of coating material. In certain embodiments, the tablet is coated with about 3, 3.5, 4, 4.5, or 5 weight percent of coating material. In certain embodiments, the tablet is coated with about 4% coating material. In certain embodiments, the tablet is coated with about 4% of a film coating. In certain embodiments, the tablet is coated with about 4% of Opadry® II white.

### Additional embodiments of the invention

A1. In certain embodiments, the advantageous fixed-dose combination comprises:
   a) Compound 1, or a pharmaceutically acceptable salt thereof,
   b) a spray-dried dispersion of ruzasvir, or a pharmaceutically acceptable salt thereof, in HPMCAS; and
   c) one or more excipients selected from a filler, a disintegrant, a glidant, a lubricant, and an osmogen, as described herein.
A2. The fixed-dose combination of embodiment A1, wherein Compound 1 or a pharmaceutically acceptable salt thereof is bemnifosbuvir.
A3. The fixed-dose combination of embodiment A2, wherein bemnifosbuvir is provided in a crystalline form.
A4. The fixed-dose combination of embodiment A2 or A3, wherein bemnifosbuvir is provided in a crystalline form characterized by three or more XRPD peaks selected from 5.2±0.2°, 7.3±0.2°, 8.9±0.2°, 13.6±0.2°, 17.0±0.2°, 19.9±0.2°, and 21.8±0.2° 2theta.
A5. The fixed-dose combination of any one of embodiments A1-A5, wherein the spray-dried dispersion of (b) comprises from about 40% to about 60% ruzasvir.
A6. The fixed-dose combination of any one of embodiments A1-A6, wherein the spray-dried dispersion of (b) comprises about 50% ruzasvir.
A7. The fixed-dose combination of any one of embodiments A1-A7, wherein the spray-dried dispersion of (b) comprises 50% ruzasvir and from about 30% to about 50% HPMCAS.
A8. The fixed-dose combination of any one of embodiments A1-A8, wherein the spray-dried dispersion of (b) comprises about 50% ruzasvir and about 50% HPMCAS.
A9. The fixed-dose combination of embodiments A7 or A8, wherein the HPMCAS is HPMCAS-L.
A10. The fixed-dose combination of any one of embodiments A1-A9, wherein the one or more excipients of (c) is a filler, a disintegrant, a glidant, a lubricant, and an osmogen.
A11. The fixed-dose combination of any one of embodiments A1-A10, wherein (c) comprises a ductile filler.
A12. The fixed-dose combination of any one of embodiments A1-A11, wherein (c) comprises microcrystalline cellulose.
A13. The fixed-dose combination of any one of embodiments A1-A10, wherein (c) comprises a brittle filler.
A14. The fixed-dose combination of any one of embodiments A1-A10 and A13, wherein (c) comprises mannitol.
A15. The fixed-dose combination of embodiment A14, wherein the mannitol is spray processed.
A16. The fixed-dose combination of any one of embodiments A1-A15, wherein (c) comprises a disintegrant.
A17. The fixed-dose combination of any one of embodiments A1-A16, wherein (c) comprises crospovidone.
A18. The fixed-dose combination of any one of embodiments A1-A17, wherein (c) comprises a glidant.
A19. The fixed-dose combination of any one of embodiments A1-A18, wherein (c) comprises silicon dioxide.
A20. The fixed-dose combination of any one of embodiments A1-A19, wherein (c) comprises a lubricant.
A21. The fixed-dose combination of any one of embodiments A1-A20, wherein (c) comprises sodium stearyl fumarate.
A22. The fixed-dose combination of any one of embodiments A1-A21, wherein (c) comprises an osmogen.
A23. The fixed-dose combination of any one of embodiments A1-A22, wherein (c) comprises sodium chloride.
A24. The fixed-dose combination of any one of embodiments A1-A23, wherein the combination is blended, compacted, and screened to form an intragranular mix.
A25. The fixed-dose combination of embodiment A24, wherein the intragranular mix is blended with one or more extragranular excipients.
A26. The fixed-dose combination of embodiment A24 or A25, wherein the intragranular mix is blended with one or more extragranular excipients selected from a disintegrant, a glidant, and a lubricant.
A27. The fixed-dose combination of any one of embodiments A24-A26, wherein the one or more extragranular excipients comprises crospovidone.
A28. The fixed-dose combination of any one of embodiments A24-A26, wherein the one or more extragranular excipients comprises silicon dioxide.
A29. The fixed-dose combination of any one of embodiments A24-A26, wherein the one or more extragranular excipients comprises sodium stearyl fumarate.
A30. The fixed-dose combination of any one of embodiments A24-A26, wherein the extragranular excipients comprise crospovidone, silicon dioxide, and sodium stearyl fumarate.
A31. The fixed-dose combination of any one of embodiments A24-A30, wherein the intragranular mix is blended with the extragranular excipients and then pressed into tablets.
A32. The fixed-dose combination of embodiment A31, wherein the tablets are further coated.
A33. The fixed-dose combination of embodiment A32, wherein the tablets are coated with a film coating.

In certain embodiments, the invention includes:
B1. An advantageous fixed-dose combination comprising:
   a) Compound 1, or a pharmaceutically acceptable salt thereof,
   b) a spray-dried dispersion of ruzasvir, or a pharmaceutically acceptable salt thereof, in an enteric polymer; and
   c) one or more excipients selected from a filler, a disintegrant, a glidant, a lubricant, and an osmogen, as described herein.
B2. An advantageous fixed-dose combination comprising
   a) Compound 1, or a pharmaceutically acceptable salt thereof,
   b) a spray-dried dispersion of ruzasvir, or a pharmaceutically acceptable salt thereof, in HPMCAS; and
   c) one or more excipients selected from a filler, a disintegrant, a glidant, a lubricant, and an osmogen, as described herein; wherein
   the fixed-dose combination provides
      (i) at least about 70% of the exposure of Compound 1, or a pharmaceutically acceptable salt thereof and ruzasvir dosed individually,
      (ii) does not contain an excipient that is an efflux inhibitor,
      (iii) an increase in exposure, or a decrease of less than about 10% in exposure when administered after a meal, and
      (iv) results in at least about 85% dissolution of Compound 1, or a pharmaceutically acceptable salt thereof, and ruzasvir within 15 minutes.
B3. The fixed-dose combination of embodiment B1 or B2, wherein the enteric polymer is hydroxypropyl methylcellulose acetate succinate (HPMCAS).
B4. The fixed-dose combination of any one of embodiments B1-B3, wherein Compound 1 or a pharmaceutically acceptable salt thereof is bemnifosbuvir.
B5. The fixed-dose combination of embodiment B4, wherein bemnifosbuvir is provided in a crystalline form.
B6. The fixed-dose combination of embodiment B4 or B5, wherein bemnifosbuvir is provided in a crystalline form characterized by three or more XRPD peaks selected from 5.2±0.2°, 7.3±0.2°, 8.9±0.2°, 13.6±0.2°, 17.0±0.2°, 19.9±0.2°, and 21.8±0.2° 2theta.
B7. The fixed-dose combination of any one of embodiments B1-B6, comprising from about 250 mg to about 300 mg of bemnifosbuvir.
B8. The fixed-dose combination of any one of embodiments B1-B7, comprising about 250 mg of bemnifosbuvir.
B9. The fixed-dose combination of any one of embodiments B1-B7, comprising about 275 mg of bemnifosbuvir.
B10. The fixed-dose combination of any one of embodiments B1-B7, comprising about 300 mg of bemnifosbuvir.
B11. The fixed-dose combination of any one of embodiments B1-B7, comprising about 325 mg of bemnifosbuvir.
B12. The fixed-dose combination of any one of embodiments B1-B7, comprising about 350 mg of bemnifosbuvir.
B13. The fixed-dose combination of any one of embodiments B1-B12, wherein the spray-dried dispersion of (b) comprises from about 40% to about 60% ruzasvir.
B14. The fixed-dose combination of any one of embodiments B1-B13, wherein the spray-dried dispersion of (b) comprises about 50% ruzasvir.
B15. The fixed-dose combination of any one of embodiments B1-B14, wherein the spray-dried dispersion of (b) comprises 50% ruzasvir and from about 30% to about 50% HPMCAS.
B16. The fixed-dose combination of any one of embodiments B1-B15, wherein the spray-dried dispersion of (b) comprises about 50% ruzasvir and about 50% HPMCAS.
B17. The fixed-dose combination of any one of embodiments B1-B16, wherein the HPMCAS is HPMCAS-L.
B18. The fixed-dose combination of any one of embodiments B1-B17, comprising from about 150 mg to about 250 mg of the spray-dried dispersion of (b).
B19. The fixed-dose combination of any one of embodiments B1-B17, comprising about 160 mg of the spray-dried dispersion of (b).
B20. The fixed-dose combination of any one of embodiments B1-B17, comprising about 180 mg of the spray-dried dispersion of (b).
B21. The fixed-dose combination of any one of embodiments B1-B17, comprising about 200 mg of the spray-dried dispersion of (b).
B22. The fixed-dose combination of any one of embodiments B1-B17, comprising about 220 mg of the spray-dried dispersion of (b).
B23. The fixed-dose combination of any one of embodiments B1-B17, comprising about 240 mg of the spray-dried dispersion of (b).
B24. The fixed-dose combination of any one of embodiments B1-B23, wherein the one or more excipients of (c) is a filler, a disintegrant, a glidant, a lubricant, and an osmogen.
B25. The fixed-dose combination of any one of embodiments B1-B24, wherein (c) comprises a ductile filler.
B26. The fixed-dose combination of any one of embodiments B1-B25, wherein (c) comprises microcrystalline cellulose.
B27. The fixed-dose combination of any one of embodiments B1-B24, wherein (c) comprises a brittle filler.
B28. The fixed-dose combination of any one of embodiments B1-B24 and B27, wherein (c) comprises mannitol.
B29. The fixed-dose combination of embodiment B28, wherein the mannitol is spray processed.
B30. The fixed-dose combination of any one of embodiments B1-B29, wherein (c) comprises a disintegrant.
B31. The fixed-dose combination of any one of embodiments B1-B30, wherein (c) comprises crospovidone.
B32. The fixed-dose combination of any one of embodiments B1-B31, wherein (c) comprises a glidant.
B33. The fixed-dose combination of any one of embodiments B1-B32, wherein (c) comprises silicon dioxide.
B34. The fixed-dose combination of any one of embodiments B1-B33, wherein (c) comprises a lubricant.
B35. The fixed-dose combination of any one of embodiments B1-B34, wherein (c) comprises sodium stearyl fumarate.
B36. The fixed-dose combination of any one of embodiments B1-B35, wherein (c) comprises an osmogen.
B37. The fixed-dose combination of any one of embodiments B1-B36, wherein (c) comprises sodium chloride.
B38. The fixed-dose combination of any one of embodiments B1-B37, wherein the combination is blended, compacted, and screened to form an intragranular mix.
B39. The fixed-dose combination of embodiment B38, wherein the intragranular mix is blended with one or more extragranular excipients.
B40. The fixed-dose combination of embodiment B38 or B39, wherein the intragranular mix is blended with one or more extragranular excipients selected from a disintegrant, a glidant, and a lubricant.
B41. The fixed-dose combination of any one of embodiments B38-B40, wherein the one or more extragranular excipients comprises crospovidone.
B42. The fixed-dose combination of any one of embodiments B38-B40, wherein the one or more extragranular excipients comprises silicon dioxide.
B43. The fixed-dose combination of any one of embodiments B38-B40, wherein the one or more extragranular excipients comprises sodium stearyl fumarate.
B44. The fixed-dose combination of any one of embodiments B38-B40, wherein the extragranular excipients comprise crospovidone, silicon dioxide, and sodium stearyl fumarate.
B45. The fixed-dose combination of any one of embodiments B38-B44, wherein the intragranular mix is blended with the extragranular excipients and then pressed into tablets.
B46. The fixed-dose combination of embodiment B45, wherein the tablets are further coated.
B47. The fixed-dose combination of embodiment B46, wherein the tablets are coated with a film coating.
B48. A fixed-dose combination comprising an intragranular mix and an extragranular mix, wherein the intragranular mix comprises
   (iv) bemnifosbuvir, in a crystalline form;
   (v) a spray-dried dispersion of ruzasvir and HPMCAS-L; and
   (vi) microcrystalline cellulose, mannitol, crospovidone, silicon dioxide, sodium stearyl fumarate, and sodium chloride;
   and the extragranular mix comprises one or more excipients selected from a disintegrant, a glidant, and a lubricant.
B49. The fixed-dose combination of embodiment B48, wherein the spray-dried dispersion of (ii) comprises from about 40% to about 60% ruzasvir and HPMCAS-L.
B50. The fixed-dose combination of embodiment B48 or B49, wherein the spray-dried dispersion of (ii) comprises about 50% ruzasvir and at least about 40% HPMCAS-L.
B51. The fixed-dose combination of any one of embodiments B48-B50, wherein the spray-dried dispersion of (ii) comprises about 50% ruzasvir and about 50% HPMCAS-L.
B52. The fixed-dose combination of any one of embodiments B48-B51, wherein the fixed-dose combination comprises from about 250 mg to about 350 mg of bemnifosbuvir, in a crystalline form.
B53. The fixed-dose combination of any one of embodiments B48-B52, wherein the fixed-dose combination comprises from about 250 mg to about 350 mg of bemnifosbuvir, in a crystalline form.
B54. The fixed-dose combination of any one of embodiments B48-B53, wherein the fixed-dose combination comprises about 250 mg of bemnifosbuvir in a crystalline form.
B55. The fixed-dose combination of any one of embodiments B48-B53, wherein the fixed-dose combination comprises about 275 mg of bemnifosbuvir, in a crystalline form.
B56. The fixed-dose combination of any one of embodiments B48-B53, wherein the fixed-dose combination comprises about 300 mg of bemnifosbuvir, in a crystalline form.
B57. The fixed-dose combination of any one of embodiments B48-B53, wherein the fixed-dose combination comprises about 325 mg of bemnifosbuvir, in a crystalline form.
B58. The fixed-dose combination of any one of embodiments B48-B53, wherein the fixed-dose combination comprises about 350 mg of bemnifosbuvir, in a crystalline form.
B59. The fixed-dose combination of any one of embodiments B48-B58, comprising from about 150 mg to about 250 mg of the spray-dried dispersion of (ii).
B60. The fixed-dose combination of any one of embodiments B48-B59, comprising about 160 mg of the spray-dried dispersion of (ii).
B61. The fixed-dose combination of any one of embodiments B48-B59, comprising about 180 mg of the spray-dried dispersion of (ii).
B62. The fixed-dose combination of any one of embodiments B48-B59, comprising about 200 mg of the spray-dried dispersion of (ii).
B63. The fixed-dose combination of any one of embodiments B48-B59, comprising about 220 mg of the spray-dried dispersion of (ii).
B64. The fixed-dose combination of any one of embodiments B48-B59, comprising about 240 mg of the spray-dried dispersion of (ii).
B65. A fixed-dose combination comprising an intragranular mix and an extragranular mix, wherein the intragranular mix comprises:

| Intragranular Mix Component | Amount (milligrams) |
|---|---|
| Bemnifosbuvir, in a | From about 250 mg to about 350 mg |
| crystalline form | |
| Spray-dried dispersion | From about 150 mg to about 250 mg |
| comprising about 50% ruzasvir | |
| and about 50% HPMCAS-L | |
| Microcrystalline cellulose | From about 300 mg to about 450 mg |
| Mannitol | From about 125 mg to about 250 mg |
| Crospovidone | From about 90 mg to about 140 mg |
| Silicon dioxide | From about 5 mg to about 35 mg |
| Sodium stearyl fumarate | From about 5 mg to about 35 mg |
| Sodium chloride | From about 50 mg to about 150 mg |

and the extragranular mix comprises one or more excipients selected from a disintegrant, a glidant, and a lubricant.
B66. A fixed-dose combination comprising an intragranular mix and an extragranular mix, wherein the intragranular mix comprises:

| Intragranular Mix Component | Amount (wt %) |
|---|---|
| Bemnifosbuvir, in a | From about 15% to about 30%, |
| crystalline form | typically from about 20% to |
| | about 25% |
| Spray-dried dispersion | From about 5% to about 25%, |
| comprising about 50% ruzasvir | typically from about 12% to |
| and about 50% HPMCAS-L | about 27% |
| Microcrystalline cellulose | From about 15% to about 30%, |
| | typically from about 22% to |
| | about 29% |
| Mannitol | From about 7% to about 20%, |
| | typically from about 10% to |
| | about 15% |
| Crospovidone | From about 5% to about 15%, |
| | typically from about 8% to |
| | about 12% |
| Silicon dioxide | From about 0.5% to about 5%, |
| | typically from about 1% to 2% |
| Sodium stearyl fumarate | From about 0.5% to about 5%, |
| | typically from about 1% to 2% |
| Sodium chloride | From about 3% to about 12%, |
| | typically from about 5% to |
| | about 10% |
| Total | 100% |

in proportions selected from the ranges such that the total is 100%; and the extragranular mix comprises one or more excipients selected from a disintegrant, a glidant, and a lubricant.
B67. A fixed-dose combination comprising an intragranular mix and an extragranular mix, wherein the intragranular mix comprises:

| Intragranular Mix Component | Amount (milligrams) |
|---|---|
| Bemnifosbuvir, in a | From about 250 mg to about 350 mg |
| crystalline form | |
| Spray-dried dispersion | From about 150 mg to about 250 mg |
| comprising about 50% ruzasvir | |
| and about 50% HPMCAS-L | |
| Microcrystalline cellulose | From about 300 mg to about 450 mg |
| Mannitol | From about 125 mg to about 250 mg |
| Crospovidone | From about 90 mg to about 140 mg |
| Silicon dioxide | From about 5 mg to about 35 mg |
| Sodium stearyl fumarate | From about 5 mg to about 35 mg |
| Sodium chloride | From about 50 mg to about 150 mg |

an the extragranular mix comprises:

| | |
|---|---|
| Crospovidone | From about 15 mg to about 45 mg |
| Silicon dioxide | From about 2 mg to about 20 mg |
| Sodium stearyl fumarate | From about 2 mg to about 20 mg |

B68. A fixed-dose combination comprising an intragranular mix and an extragranular mix, wherein the intragranular mix comprises:

| Intragranular Mix Component | Amount (wt %) |
|---|---|
| Bemnifosbuvir, in a | From about 15% to about 30%, |
| crystalline form | typically from about 20% to |
| | about 25% |
| Spray-dried dispersion | From about 5% to about 25%, |
| comprising about 50% ruzasvir | typically from about 12% to |
| and about 50% HPMCAS-L | about 27% |
| Microcrystalline cellulose | From about 15% to about 30%, |
| | typically from about 22% to |
| | about 29% |
| Mannitol | From about 7% to about 20%, |
| | typically from about 10% to |
| | about 15% |
| Crospovidone | From about 5% to about 15%, |
| | typically from about 8% to |
| | about 12% |
| Silicon dioxide | From about 0.5% to about 5%, |
| | typically from about 1% to 2% |
| Sodium stearyl fumarate | From about 0.5% to about 5%, |
| | typically from about 1% to 2% |
| Sodium chloride | From about 3% to about 12%, |
| | typically from about 5% to |
| | about 10% |
| Total Intragranular | From about 90% to about 99% |

an the extragranular mix comprises:

| | |
|---|---|
| Crospovidone | From about 0.5% to about 5%, |
| | typically from about 1% to 3% |
| Silicon dioxide | From about 0.05% to about 2%, |
| | typically from about 0.1% to 1% |
| Sodium stearyl fumarate | From about 0.05% to about 2%, |
| | typically from about 0.1% to 1% |
| Total Extragranular | From about 1% to about 10% |

in proportions selected from the ranges such that the total of Intragranular components and Extragranular components is 100%.
B69. The fixed-dose combination of any one of embodiments B48-B68, wherein the intragranular mix is blended with the extragranular excipients and then pressed into tablets.
B70. The fixed-dose combination of embodiment B69, wherein the tablets are further coated.
B71. The fixed-dose combination of embodiment B70, wherein the tablets are coated with a film coating.
B72. A method of treating HCV in a human patient in need thereof comprising administering the fixed-dose combination of any one of embodiments B1-B71.
B73. The method of embodiment B72, wherein the fixed-dose combination is administered once per day.
B74. The method of embodiment B72, wherein the fixed-dose combination is administered twice per day.
B75. The method of embodiment B72, wherein the fixed-dose combination is administered three times per day.
B76. The method of embodiment B72, wherein the fixed-dose combination is administered four times per day.
B77. The method of embodiment B72, wherein the fixed-dose combination is administered as two oral dosage forms once per day.
B78. The method of embodiment B72, wherein the fixed-dose combination is administered as two oral dosage forms twice per day.
B79. The method of embodiment B72, wherein the fixed-dose combination is administered as two oral dosage forms three times per day.
B80. The method of any one of embodiments B72-B79, wherein the fixed-dose combination is administered for about four to about sixteen weeks.
B81. The method of any one of embodiments B72-B79, wherein the fixed-dose combination is administered for about six to about twelve weeks.
B82. The method of any one of embodiments B72-B79, wherein the fixed-dose combination is administered for at least four weeks.
B83. The method of any one of embodiments B72-B79, wherein the fixed-dose combination is administered for at least five weeks.
B84. The method of any one of embodiments B72-B79, wherein the fixed-dose combination is administered for at least six weeks.
B85. The method of any one of embodiments B72-B79, wherein the fixed-dose combination is administered for at least seven weeks.
B86. The method of any one of embodiments B72-B79, wherein the fixed-dose combination is administered for at least eight weeks.
B87. The method of any one of embodiments B72-B79, wherein the fixed-dose combination is administered for at least nine weeks.
B88. The method of any one of embodiments B72-B79, wherein the fixed-dose combination is administered for at least ten weeks.
B89. The method of any one of embodiments B72-B79, wherein the fixed-dose combination is administered for at least eleven weeks.
B90. The method of any one of embodiments B72-B79, wherein the fixed-dose combination is administered for at least twelve weeks.

In alternative embodiments, a fixed-dose composition prepared from bemnifosbuvir and ruzasvir or a pharmaceutically acceptable salt thereof, also comprises one or more of the following excipients: a phosphoglyceride; phosphatidylcholine; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohol such as polyethylene glycol (PEG); polyoxyethylene-9-lauryl ether; a surface active fatty acid, such as palmitic acid or oleic acid; fatty acid; fatty acid monoglyceride; fatty acid diglyceride; fatty acid amide; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebroside; dicetylphosphate; dipalmitoylphosphatidylglycerol; stearylamine; dodecylamine; hexadecyl-amine; acetyl palmitate; glycerol ricinoleate; hexadecyl stearate; isopropyl myristate; tyloxapol; poly(ethylene glycol)5000-phosphatidylethanolamine; poly(ethylene glycol)400-monostearate; phospholipid; synthetic and/or natural detergent having high surfactant properties; deoxycholate; cyclodextrin; chaotropic salt; ion pairing agent; glucose, fructose, galactose, ribose, lactose, sucrose, maltose, trehalose, cellbiose, mannose, xylose, arabinose, mannuronic acid, glucosamine, galatosamine, and neuramic acid; pullulan, cellulose, microcrystalline cellulose, silicified microcrystalline cellulose, hydroxypropyl methylcellulose (HPMC), hydroxycellulose (HC), methylcellulose (MC), dextran, glycogen, hydroxyethylstarch, glycon, amylose, chitosan, N,O-carboxylmethylchitosan, algin and alginic acid, starch, chitin, inulin, konjac, pustulan, heparin, hyaluronic acid, curdlan, and xanthan, mannitol, sorbitol, xylitol, erythritol, maltitol, and lactitol, a pluronic polymer, polyethylene, polycarbonate (e.g., poly(1,3-dioxan-2one)), polyanhydride (e.g., poly(sebacic anhydride)), polypropylfumerate, polyamide (e.g. polycaprolactam), polyacetal, polyether, polyester (e.g., polylactide, polyglycolide, polylactide-co-glycolide, polycaprolactone, polyhydroxyacid (e.g., poly((β-hydroxyalkanoate))), poly(orthoester), polycyanoacrylate, polyvinyl alcohol, polyurethane, polyphosphazene, polyacrylate, polymethacrylate, polyurea, polystyrene, and polyamine, polylysine, polylysine-PEG copolymer, and poly(ethyleneimine), poly(ethylene imine)-PEG copolymer, glycerol monocaprylocaprate, propylene glycol, Vitamin E TPGS (also known as d-α-Tocopheryl polyethylene glycol 1000 succinate), gelatin, titanium dioxide, polyvinylpyrrolidone (PVP), hydroxypropyl methyl cellulose (HPMC), hydroxypropyl cellulose (HPC), methyl cellulose (MC), block copolymers of ethylene oxide and propylene oxide (PEO/PPO), polyethyleneglycol (PEG), sodium carboxymethylcellulose (NaCMC), or hydroxypropylmethyl cellulose acetate succinate (HPMCAS).

In alternative embodiments, a fixed-dose composition prepared from bemnifosbuvir and ruzasvir or a pharmaceutically acceptable salt thereof, also comprises one or more of the following surfactants: polyoxyethylene glycol, polyoxypropylene glycol, decyl glucoside, lauryl glucoside, octyl glucoside, polyoxyethylene glycol octylphenol, Triton X-100, glycerol alkyl ester, glyceryl laurate, cocamide MEA, cocamide DEA, dodecyldimethylamine oxide, and poloxamers. Examples of poloxamers include, poloxamers 188, 237, 338 and 407. These poloxamers are available under the trade name Pluronic® (available from BASF, Mount Olive, N.J.) and correspond to Pluronic® F-68, F-87, F-108 and F-127, respectively. Poloxamer 188 (corresponding to Pluronic® F-68) is a block copolymer with an average molecular mass of about 7,000 to about 10,000 Da, or about 8,000 to about 9,000 Da, or about 8,400 Da. Poloxamer 237 (corresponding to Pluronic® F-87) is a block copolymer with an average molecular mass of about 6,000 to about 9,000 Da, or about 6,500 to about 8,000 Da, or about 7,700 Da. Poloxamer 338 (corresponding to Pluronic® F-108) is a block copolymer with an average molecular mass of about 12,000 to about 18,000 Da, or about 13,000 to about 15,000 Da, or about 14,600 Da. Poloxamer 407 (corresponding to Pluronic® F-127) is a polyoxyethylene-polyoxypropylene triblock copolymer in a ratio of between about E101 P56 E101 to about E106 P70 E106, or about E101 P56E101, or about E106 P70 E106, with an average molecular mass of about 10,000 to about 15,000 Da, or about 12,000 to about 14,000 Da, or about 12,000 to about 13,000 Da, or about 12,600 Da.

In alternative embodiments, a fixed-dose composition prepared bemnifosbuvir and ruzasvir or a pharmaceutically acceptable salt thereof, also comprises one or more of the following surfactants: polyvinyl acetate, cholic acid sodium salt, dioctyl sulfosuccinate sodium, hexadecyltrimethyl ammonium bromide, saponin, sugar esters, Triton X series, sorbitan trioleate, sorbitan mono-oleate, polyoxyethylene (20) sorbitan monolaurate, polyoxyethylene (20) sorbitan monooleate, oleyl polyoxyethylene (2) ether, stearyl polyoxyethylene (2) ether, lauryl polyoxyethylene (4) ether, block copolymers of oxyethylene and oxypropylene, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, cetylpyridinium chloride, benzalkonium chloride, olive oil, glyceryl monolaurate, corn oil, cotton seed oil, and sunflower seed oil.

In alternative embodiments, a fixed-dose composition prepared from bemnifosbuvir and ruzasvir or a pharmaceutically acceptable salt thereof, is prepared by a process that includes solvent or dry granulation optionally followed by compression or compaction, spray drying, nano-suspension processing, hot melt extrusion, extrusion/spheronization, molding, spheronization, layering (e.g., spray layering suspension or solution), or the like. Examples of such techniques include direct compression, using appropriate punches and dies, for example wherein the punches and dies are fitted to a suitable tableting press; wet granulation using suitable granulating equipment such as a high shear granulator to form wetted particles to be dried into granules; granulation followed by compression using appropriate punches and dies, wherein the punches and dies are fitted to a suitable tableting press; extrusion of a wet mass to form a cylindrical extrudate to be cut into desire lengths or break into lengths under gravity and attrition; extrusion/spheronization where the extrudate is rounded into spherical particles and densified by spheronization; spray layering of a suspension or solution onto an inert core using a technique such as a convention pan or Wurster column; injection or compression molding using suitable molds fitted to a compression unit; and the like.

Exemplary disintegrants include alginic acid, carboxymethylcellulose calcium, carboxymethylcellulose sodium, cross-linked sodium carboxymethylcellulose (sodium croscarmellose), powdered cellulose, chitosan, croscarmellose sodium, crospovidone, guar gum, low substituted hydroxypropyl cellulose, methyl cellulose, microcrystalline cellulose, sodium alginate, sodium starch glycolate, partially pregelatinized starch, pregelatinized starch, starch, sodium carboxymethyl starch, and the like, or a combination thereof.

Exemplary lubricants include calcium stearate, magnesium stearate, glyceryl behenate, glyceryl palmitostearate, hydrogenated castor oil, light mineral oil, sodium lauryl sulfate, magnesium lauryl sulfate, sodium stearyl fumarate, stearic acid, zinc stearate, silicon dioxide, colloidal silicon dioxide, dimethyldichlorosilane treated with silica, talc, or a combination thereof.

The dosage form cores described herein may be coated to result in coated tablets. The dosage from cores can be coated with a functional or non-functional coating, or a combination of functional and non-functional coatings. “Functional coating” includes tablet coatings that modify the release properties of the total composition, for example, a sustained-release or delayed-release coating. “Non-functional coating” includes a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition. A non-functional coating can also mask the taste of the uncoated composition including the active pharmaceutical ingredient. A coating may comprise a light blocking material, a light absorbing material, or a light blocking material and a light absorbing material.

Exemplary polymethacrylates include copolymers of acrylic and methacrylic acid esters, such as a. an aminomethacrylate copolymer USP/NF such as a poly(butyl methacrylate, (2-dimethyl aminoethyl)methacrylate, methyl methacrylate) 1:2:1 (e.g., EUDRAGIT E 100, EUDRAGIT EPO, and EUDRAGIT E 12.5; CAS No. 24938-16-7); b. a poly(methacrylic acid, ethyl acrylate) 1:1 (e.g., EUDRAGIT L30 D-55, EUDRAGIT L100-55, EASTACRYL 30D, KOLLICOAT MAE 30D AND 30DP; CAS No. 25212-88-8); c. a poly(methacrylic acid, methyl methacrylate) 1:1 (e.g., EUDRAGIT L 100, EUDRAGIT L 12.5 and 12.5 P; also known as methacrylic acid copolymer, type ANF; CAS No. 25806-15-1); d. a poly(methacrylic acid, methyl methacrylate) 1:2 (e.g., EUDRAGIT S 100, EUDRAGIT S 12.5 and 12.5P; CAS No. 25086-15-1); e. a poly(methyl acrylate, methyl methacrylate, methacrylic acid) 7:3:1 (e.g., Eudragit FS 30 D; CAS No. 26936-24-3); f. a poly(ethyl acrylate, methylmethacrylate, trimethylammonioethyl methacrylate chloride) 1:2:0.2 or 1:2:0.1 (e.g., EUDRAGITS RL 100, RL PO, RL 30 D, RL 12.5, RS 100, RS PO, RS 30 D, or RS 12.5; CAS No. 33434-24-1); g. a poly(ethyl acrylate, methyl methacrylate) 2:1 (e.g., EUDRAGIT NE 30 D, Eudragit NE 40D, Eudragit NM 30D; CAS No. 9010-88-2); and the like, or a combination thereof.

Suitable alkylcelluloses include, for example, methylcellulose, ethylcellulose, and the like, or a combination thereof. Exemplary water based ethylcellulose coatings include AQUACOAT, a 30% dispersion further containing sodium lauryl sulfate and cetyl alcohol, available from FMC, Philadelphia, PA; SURELEASE a 25% dispersion further containing a stabilizer or other coating component (e.g., ammonium oleate, dibutyl sebacate, colloidal anhydrous silica, medium chain triglycerides, etc.) available from Colorcon, West Point, PA; ethyl cellulose available from Aqualon or Dow Chemical Co (Ethocel), Midland, MI. Those skilled in the art will appreciate that other cellulosic polymers, including other alkyl cellulosic polymers, can be substituted for part or all of the ethylcellulose.

Other suitable materials that can be used to prepare a functional coating include hydroxypropyl methylcellulose acetate succinate (HPMCAS); cellulose acetate phthalate (CAP); a polyvinylacetate phthalate; neutral or synthetic waxes, fatty alcohols (such as lauryl, myristyl, stearyl, cetyl or specifically cetostearyl alcohol), fatty acids, including fatty acid esters, fatty acid glycerides (mono-, di-, and tri-glycerides), hydrogenated fats, hydrocarbons, normal waxes, stearic acid, stearyl alcohol, hydrophobic and hydrophilic materials having hydrocarbon backbones, or a combination thereof. Suitable waxes include beeswax, glycowax, castor wax, carnauba wax, microcrystalline wax, candelilla, and wax-like substances, e.g., material normally solid at room temperature and having a melting point of from about 30 °C to about 100 °C, or a combination thereof.

In other embodiments, a functional coating may include digestible, long chain (e.g., C8-C50, specifically C12-C40), substituted or unsubstituted hydrocarbons, such as fatty acids, fatty alcohols, glyceryl esters of fatty acids, mineral and vegetable oils, waxes, or a combination thereof. Hydrocarbons having a melting point of between about 25 °C and about 90 °C may be used. Specifically, long chain hydrocarbon materials, fatty (aliphatic) alcohols can be used.

The coatings can optionally contain additional pharmaceutically acceptable excipients such as a plasticizer, a stabilizer, a water-soluble component (e.g., pore formers), an anti-tacking agent (e.g., talc), a surfactant, and the like, or a combination thereof.

A functional coating may include a release-modifying agent, which affects the release properties of the functional coating. The release-modifying agent can, for example, function as a pore-former or a matrix disrupter. The release-modifying agent can be organic or inorganic, and include materials that can be dissolved, extracted or leached from the coating in the environment of use. The release-modifying agent can comprise one or more hydrophilic polymers including cellulose ethers and other cellulosics, such as hydroxypropyl methylcellulose, hydroxypropylcellulose, hydroxyethylcellulose, methyl cellulose, cellulose acetate phthalate, or hydroxypropyl methylcellulose acetate phthalate; povidone; polyvinyl alcohol; an acrylic polymer, such as gastric soluble Eudragit FS 30D, Ph sensitive Eudragit L30D 55, L 100, S 100, or L 100-55; or a combination thereof. Other exemplary release-modifying agents include a povidone; a saccharide (e.g., lactose, and the like); a metal stearate; an inorganic salt (e.g., dibasic calcium phosphate, sodium chloride, and the like); a polyethylene glycol (e.g., polyethylene glycol (PEG) 1450, and the like); a sugar alcohol (e.g., sorbitol, mannitol, and the like); an alkali alkyl sulfate (e.g., sodium lauryl sulfate); a polyoxyethylene sorbitan fatty acid ester (e.g., polysorbate); or a combination thereof. Exemplary matrix disrupters include water insoluble organic or inorganic material. Organic polymers including but not limited to cellulose, cellulose ethers such as ethylcellulose, cellulose esters such as cellulose acetate, cellulose acetate butyrate and cellulose acetate propionate; and starch can function as matrix disrupters. Examples or inorganic disrupters include many calcium salts such as mono-, di- and tri calcium phosphate; silica and, talc.

The coating may optionally contain a plasticizer to improve the physical properties of the coating. For example, because ethylcellulose has a relatively high glass transition temperature and does not form flexible films under normal coating conditions, it may be advantageous to add plasticizer to the ethylcellulose before using the same as a coating material. Generally, the amount of plasticizer included in a coating solution is based on the concentration of the polymer, e.g., can be from about 1% to about 200% depending on the polymer but is most often from about 1 wt % to about 100 wt % of the polymer. Concentrations of the plasticizer, however, can be determined by routine experimentation.

Examples of plasticizers for ethylcellulose and other celluloses include plasticizers such as dibutyl sebacate, diethyl phthalate, triethyl citrate, tributyl citrate, triacetin, or a combination thereof, although it is possible that other water-insoluble plasticizers (such as acetylated monoglycerides, phthalate esters, castor oil, etc.) can be used.

Examples of plasticizers for acrylic polymers include citric acid esters such as triethyl citrate NF, tributyl citrate, dibutyl phthalate, 1,2-propylene glycol, polyethylene glycols, propylene glycol, diethyl phthalate, castor oil, triacetin, or a combination thereof, although it is possible that other plasticizers (such as acetylated monoglycerides, phthalate esters, castor oil, etc.) can be used.

Suitable methods can be used to apply the coating material to the surface of the dosage form cores. Processes such as simple or complex coacervation, interfacial polymerization, liquid drying, thermal and ionic gelation, spray drying, spray chilling, fluidized bed coating, pan coating, or electrostatic deposition may be used.

In certain embodiments, an optional intermediate coating is used between the dosage form core and an exterior coating. Such an intermediate coating can be used to protect the active agent or other component of the core subunit from the material used in the exterior coating or to provide other properties. Exemplary intermediate coatings typically include water-soluble film forming polymers. Such intermediate coatings may include film forming polymers such as hydroxyethyl cellulose, hydroxypropyl cellulose, gelatin, hydroxypropyl methylcellulose, polyethylene glycol, polyethylene oxide, and the like, or a combination thereof; and a plasticizer. Plasticizers can be used to reduce brittleness and increase tensile strength and elasticity. Exemplary plasticizers include polyethylene glycol propylene glycol and glycerin.

### Methods of Treatment

Treatment, as used herein, refers to the administration of the combination of the advantageous fixed-dose combination of the present invention in an effective amount to a host, for example a human, that is or may become infected with an HCV virus. In one embodiment the method of treatment comprises administration of a fixed-dose combination described herein, to a host, for example a human that is or may become infected with a HCV virus.

The term “prophylactic” or preventative, when used, refers to the administration of a fixed-dose combination of the present invention to prevent or reduce the likelihood of an occurrence of the viral disorder. The present invention includes in an alternative embodiment, treatment and prophylactic or preventative therapies. In one embodiment, the fixed-dose combination is administered to a host who has been exposed to and thus is at risk of infection by a hepatitis C virus infection.

The invention includes a method of treatment of a hepatitis C virus, by administering an effective amount of the fixed-dose combination of Compound 1 (such as bemnifosbuvir) and ruzasvir including drug resistant and multidrug resistant forms of HCV and related disease states, conditions, or complications of an HCV infection, including cirrhosis and related hepatotoxicities, as well as other conditions that are secondary to an HCV infection, such as weakness, loss of appetite, weight loss, breast enlargement (especially in men), rash (especially on the palms), difficulty with clotting of blood, spider-like blood vessels on the skin, confusion, coma (encephalopathy), buildup of fluid in the abdominal cavity (ascites), esophageal varices, portal hypertension, kidney failure, enlarged spleen, decrease in blood cells, anemia, thrombocytopenia, jaundice, and hepatocellular cancer, among others. The method comprises administering to a host in need thereof, typically a human, an effective amount of the fixed-dose combination described herein, optionally in combination with at least one additional bioactive agent, for example, an additional anti-HCV agent, further optionally in combination with a pharmaceutically acceptable carrier additive and/or excipient. In another embodiment the method comprises administering to a patient at risk of an HCV infection, an effective amount of a fixed-dose combination of the present invention. In another embodiment the fixed-dose combination as described above is used with a pharmaceutically acceptable carrier, additive, or excipient, optionally in combination with a third anti-HCV agent. In another embodiment, the fixed-dose combination of the present invention can be administered to a patient after a hepatitis-related liver transplantation to protect the new organ.

The fixed-dose combination can also be used to treat conditions related to or occurring as a result of an HCV viral exposure. For example, the fixed-dose combination can be used to treat HCV antibody-positive and HCV antigen-positive conditions, viral-based chronic liver inflammation, liver cancer resulting from advanced hepatitis C (e.g., hepatocellular carcinoma), cirrhosis, acute hepatitis C, fulminant hepatitis C, chronic persistent hepatitis C, and anti-HCV-based fatigue. The fixed-dose combination can also be used prophylactically to prevent or restrict the progression of clinical illness in individuals who are anti-HCV antibody- or antigen-positive or who have been exposed to hepatitis C.

### Combination and Alternation Therapy

Drug resistance sometimes occurs by mutation of a gene that encodes for an enzyme used in viral replication. The efficacy of a combination therapy against an HCV infection, can be prolonged, augmented, or restored by adding an additional compound to the fixed-dose combination therapy. The fixed-dose combination may be administered together or in alternation with another, and perhaps even two or three other, antiviral compounds that induce a different mutation or act through a different pathway, from that of the principal combination. Alternatively, the pharmacokinetic, bio-distribution, half-life, or other parameter of the combination can be altered by such combination therapy (which may include alternation therapy if considered concerted).

This invention already provides an advantageous fixed-dose combination therapy for the treatment of HCV, or a disorder associated with an HCV infection, by administering a selected NS5B inhibitor with an NS5A inhibitor. Additional therapeutic effects may be achieved by adding a third, fourth, or even fifth active agent either co-formulated or provided separately.

Since the fixed-dose combination comprises an NS5B inhibitor (Compound 1, or a pharmaceutically acceptable salt thereof, e.g. bemnifosbuvir) and an NS5A inhibitor (ruzasvir, or a pharmaceutically acceptable salt thereof) the fixed-dose combination can be administered to a host in combination or alternation with, for example, a
(1) Protease inhibitor, such as an NS3/4A protease inhibitor;
(2) Another NS5A inhibitor;
(3) Another NS5B polymerase inhibitor;
(4) NS5B non-substrate inhibitor;
(5) Interferon alfa-2a, which may be pegylated or otherwise modified, and/or ribavirin;
(6) Non-substrate-based inhibitor;
(7) Helicase inhibitor;
(8) Antisense oligodeoxynucleotide (S-ODN);
(9) Aptamer;
(10) Nuclease-resistant ribozyme;
(11) Irna, including microRNA and SiRNA;
(12) Antibody, partial antibody or domain antibody to the virus, or
(13) Viral antigen or partial antigen that induces a host antibody response.

Non limiting examples of additional anti-HCV agents that can be administered in further combination or alternation with the combination of the present invention, include
(i) protease inhibitors such as telaprevir (Incivek®), boceprevir (Victrelis™), simeprevir (Olysio™), paritaprevir (ABT-450), glecaprevir (ABT-493), ritonavir (Norvir), ACH-2684, AZD-7295, BMS-791325, danoprevir, Filibuvir, GS-9256, GS-9451, MK-5172, Setrobuvir, Sovaprevir, Tegobuvir, VX-135, VX-222, ALS-220, and voxilaprevir.
(ii) NS5A inhibitor such as ACH-2928, ACH-3102, IDX-719, daclatasvir, ledispasvir, velpatasvir (Epclusa), elbasvir (MK-8742), grazoprevir (MK-5172), and Ombitasvir (ABT-267);
(iii) NS5B inhibitors such as AZD-7295, Clemizole, dasabuvir (Exviera), ITX-5061, PPI-461, PPI-688, sofosbuvir (Sovaldi®), MK-3682, and mericitabine;
(iv) NS5B inhibitors such as ABT-333, and MBX-700;
(v) Antibody such as GS-6624;
(vi) Combination drugs such as Harvoni (ledipasvir/sofosbuvir); Viekira Pak (ombitasvir/paritaprevir/ritonavir/dasabuvir); Viekirax (ombitasvir/paritaprevir/ritonavir); G/P (paritaprevir and glecaprevir); Technivie™ (ombitasvir/paritaprevir/ritonavir), Epclusa (sofosbuvir/velpatasvir), Zepatier (elbasvir and grazoprevir), Mavyret (glecaprevir and pibrentasvir), and Vosevi (Sofosbuvir, velpatasvir, and voxilaprevir).

If the combination is administered to treat advanced hepatitis C virus that may lead, or has led, to liver cancer or cirrhosis, in one embodiment, the compound can be administered in combination or alternation with another drug that is typically used to treat hepatocellular carcinoma (HCC), for example, as described by Andrew Zhu in “New Agents on the Horizon in Hepatocellular Carcinoma” Therapeutic Advances in Medical Oncology, V 5(1), January 2013, 41-50. Examples of suitable compounds for combination therapy where the host has or is at risk of HCC include anti-angiogenic agents, sunitinib, brivanib, linifanib, ramucirumab, bevacizumab, cediranib, pazopanib, TSU-68, 81envatinib, antibodies against EGFR, mTor inhibitors, MEK inhibitors, and histone decetylace inhibitors, capecitabine, cisplatin, carboplatin, doxorubicin, 5-fluorouracil, gemcitabine, irinotecan, oxaliplatin, topotecan, and other topoisomerases.

### General Methods

¹H, ¹⁹F and ³¹P NMR spectra were recorded on a 400 MHz Fourier transform Brucker spectrometer. Spectra were obtained DMSO-d₆ unless stated otherwise. The spin multiplicities are indicated by the symbols s (singlet), d (doublet), t (triplet), m (multiplet) and, br (broad). Coupling constants (J) are reported in Hz. The reactions were generally carried out under a dry nitrogen atmosphere using Sigma-Aldrich anhydrous solvents. All common chemicals were purchased from commercial sources.

The following abbreviations are used in the Examples:
BID: Twice a day
DCM: Dichloromethane
EtOAc: Ethyl acetate
EtOH: Ethanol
GT: Genotype
HPLC: High pressure liquid chromatography
LD: Loading dose
NaOH: Sodium hydroxide
Na₂SO₄: Sodium sulphate (anhydrous)
MeOH: Methanol
Na₂SO₄: Sodium sulfate
NH₄Cl: Ammonium chloride
PE: Petroleum ether
Silica gel (230 to 400 mesh, Sorbent)
t-BuMgCl: t-Butyl magnesium chloride
THF: Tetrahydrofuran (THF), anhydrous
TP: Triphosphate

### Example 1. Synthesis of Compound 1 and Bemnifosbuvir

### Part A: Synthesis of (2R,3R,4R,5R)-5-(2-amino-6-(methylamino)-9H-purin-9-yl)-4-fluoro-2-(hydroxymethyl)-4-methyltetrahydrofuran-3-ol (1-7)

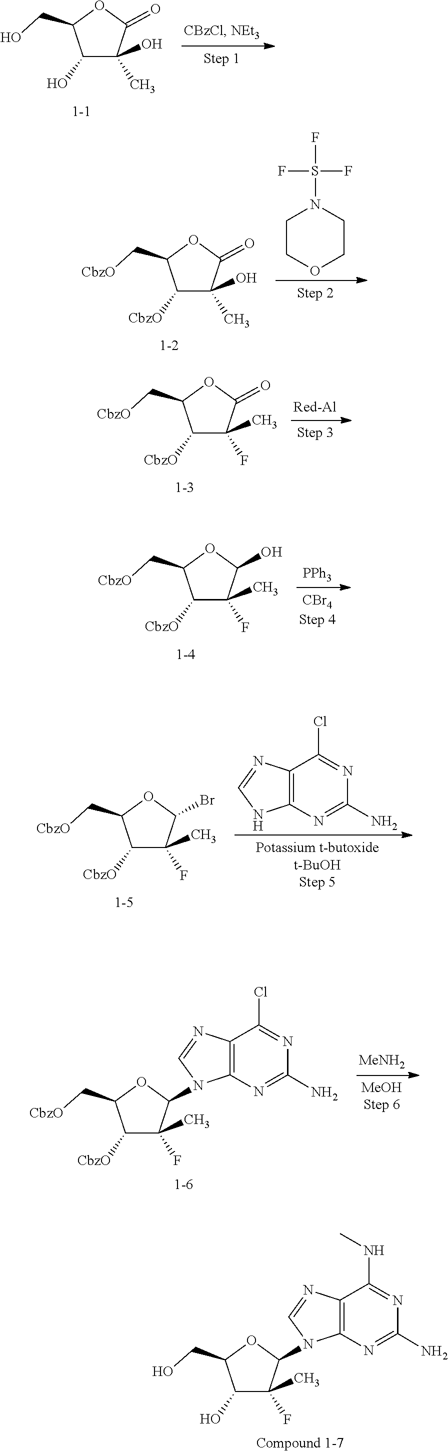

In Step 1, Compound 1-1 is dissolved in DCM and the reaction is cooled to 10 °C before benzyl chloroformate is added followed by Net₃. The reaction is allowed to warm to room temperature and stir for 12-14 hours. Following appropriate work-up and purification conditions, Compound 1-2 is isolated. In Step 2, Compound 1-2 is dissolved in acetonitrile and cooled to −15 to 5 °C before Morpho DAST is added. The reaction is allowed to stir for 6 hours. Following appropriate work-up and purification conditions, Compound 1-3 is isolated. In Step 3, Compound 1-3 is dissolved in toluene and the reaction is cooled to 0-10 °C before Red A1 is added. Following appropriate work-up and purification conditions, Compound 1-4 is isolated as the diastereomer with CAN-stereochemistry at the hydroxyl position. In Step 4, Compound 1-4 is dissolved in acetonitrile and cooled to −15 to 5 °C before CBr₄ and PPh₃ are added. Following appropriate work-up and purification conditions, Compound 1-5 is isolated. In Step 5, Compound 1-5 is dissolved is acetonitrile and t-BuOH, t-BuOK, and 6-chloro-9H-purin-2-amine are added. The reaction is heated to 40-50 °C. Following appropriate work-up and purification conditions, Compound 1-6 is isolated. In Step 6, Compound 1-6 is dissolved in MeOH and MeNH₂ is added. The reaction is heated to 20-30 C. Following appropriate work-up and purification conditions, Compound 1-7 is isolated.

### Example 2A: Synthesis of Dihydroquinine Salt of Isopropyl (hydroxy(phenoxy)phosphoryl)-L-alaninate (1-12)

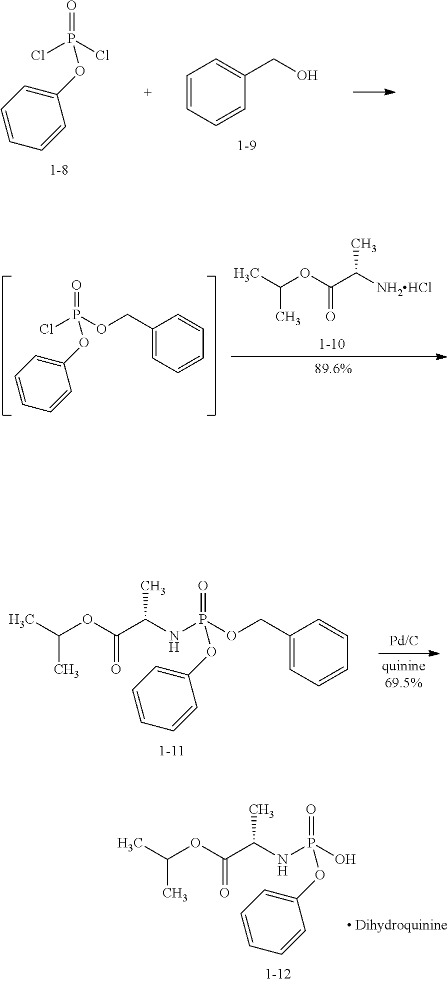

Phenyl dichlorophosphate (1-8, 150 g, 1.0 eq.) was added into 1300 Ml of isopropyl acetate. The solution was cooled to −10 °C±5 °C and then a solution of benzyl alcohol (1-9, 80.6 g, 1.05 eq.) and Et₃N (86.3 g, 1.2 eq.) was added. The mixture was stirred for 3 hours at −10±5 °C. The end point of reaction was monitored by TLC.

L-Alanine isopropyl ester hydrochloride (1-10, 125 g, 1.05 eq.) and Et₃N (152 g, 2.1 eq.) were added at −10 °C±5 °C. The reaction mixture was stirred at −10±5 °C for 2 hours. The end point of reaction was monitored by TLC.

The reaction mixture was filtered, and the filter cake was washed with 20 Ml of isopropyl acetate. The filtrate was washed with 1N HCl, water, and aqueous sodium bicarbonate. The separated organic layer was dried with anhydrous Na₂SO₄ and then concentrated to dryness under vacuum at 40 °C-50 °C to give 240 g of crude product 1-11 as a diastereomeric mixture (approximately, 1:1). (Pale yellow oil; yield: 89.6% mol/mol; HPLC purity: 83.4% by area; HPLC assay: 86.2% w/w). The product contained around 6%-7% residual benzyl alcohol. The crude 1-4 was used directly in the next step.

Compound 1-11 (135 g, 1.0 eq., 86.2% assay) and quinine (100 g, 1.0 eq.) were added into 650 Ml of i-PrOH. After 5% Pd/C (19.2 g, 60% water by KF) was added, hydrogenation was performed at 20 °C-25 °C for 8 hours using a hydrogen bag in a closed system. After completion of reaction, the mixture was filtered through a Buchner funnel. The filtrate was concentrated under vacuum to remove the solvent.

To the above residue, 300 Ml of TBME was added. The mixture was concentrated to remove the solvent under vacuum at 40 °C-45 °C, and then this step was repeated with another 300 Ml of MTBE. To the above, 600 Ml of MTBE was added, and the mixture was stirred at 40 °C-45 °C for 1 hour and then stirred at 0 °C-5 °C for additional 1 hour. The mixture was filtered, and the filter cake was washed with 100 Ml of MTBE. The cake was dried at 45 °C for 16 hours without vacuum to give 152 g of the dihydroquinine salt of isopropyl (hydroxy(phenoxy)phosphoryl)-L-alaninate (1-12, white solid; yield: 69.5% mol/mol; HPLC Purity: 97.91%).

### Example 2B: Synthesis of Dihydroquinine Salt of Isopropyl ((2-fluorophenoxy)(hydroxy)phosphoryl)-L-alaninate (1-13)

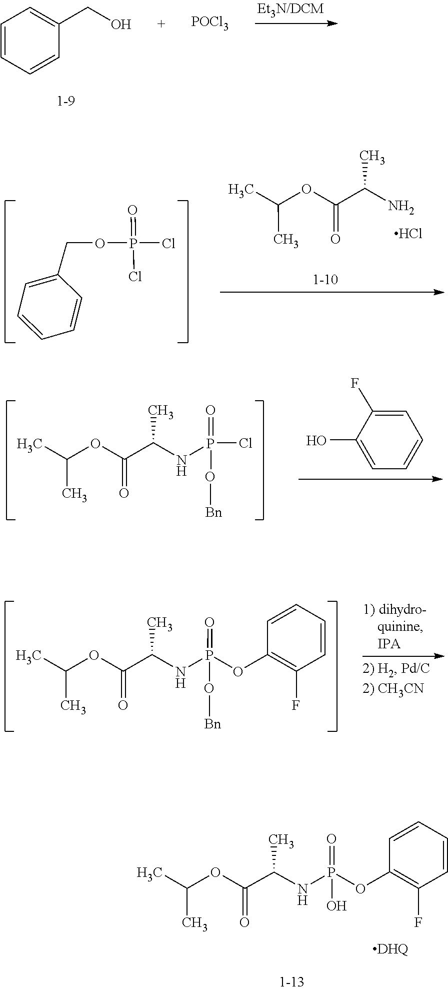

Dichloromethane (2.0 L, 10 vol) was charged to a three-necked flask at 10 °C. The flask was evacuated and refilled with nitrogen gas three times. Phosphorous oxychloride (200 g, 1.0 equiv) was charged to the flask, before the mixture was cooled to −55 °C. Dichloromethane (200 Ml, 1 vol), benzyl alcohol (141 g, 1 equiv), and triethylamine (139 g, 1.05 equiv) were added to a mixing flask at 10 °C, before charging the resultant mixture to the phosphorous oxychloride solution over 40 minutes under nitrogen protection. The reaction mixture was stirred for 1 hour at −55 °C, during which time white solids precipitated. The reaction appeared to be 99.6% completed, by HPLC analysis of a reaction mixture sample derivatized with benzylamine. L-Alanine isopropyl ester hydrochloride (219 g, 1 equiv) was charged to the reaction mixture, followed by triethylamine (271 g, 2.05 equiv) over 30 minutes.

The resultant reaction mixture was stirred for 1-2 hours, then analyzed for consumption of the first intermediate by HPLC analysis of a sample derivatized with benzylamine. The reaction appeared to be 97.7% complete.

Dichloromethane (400 Ml, 2 vol), 2-fluorophenol (146 g, 1 equiv), and triethylamine (139 g, 1.05 eq, added over 20 minutes) were combined in a mixing flask at 10 °C, before addition to the reaction mixture over 43 minutes. The batch was warmed from −55 °C to 10 °C over a period of 3 hours, then stirred 2-4 hours. HPLC analysis of a sample derivatized with benzylamine showed 2.0% area of the second intermediate. The batch was filtered, and the filter cake was rinsed with dichloromethane (400 Ml, 2 vol). Water (600 Ml, 3 vol) was charged to the filtrate, and the mixture was stirred at 10 °C for 5 minutes. The mixture was allowed to settle, and the layers were separated. Hydrochloric acid in water (3.7% w/w, 1 L, 5 vol) was charged to the organics and the mixture was stirred for 5 minutes at 10 °C. The mixture was allowed to settle, and the layers were separated. Sodium bicarbonate in water (5% w/w, 1 L, 5 vol) was charged to the organics and the mixture was stirred for 5 minutes at 5 °C. The mixture was allowed to settle, and the layers were separated. Water (600 Ml, 3 vol) was charged to the organics, and the mixture was stirred at 10 °C for 5 minutes. The mixture was allowed to settle, and the layers were separated. Charcoal (10 g, 5% w/w) was charged to the organics, and the resulting suspension was stirred for 2 hours at 10 °C. The suspension was filtered to remove the charcoal, and the filter was rinsed with dichloromethane (100 Ml). The filtrate was concentrated under vacuum at 45 °C to a volume of about 600 Ml (3 vol). Isopropanol (600 Ml, 3 vol) was charged to the mixture, which was again concentrated to about 600 Ml (3 vol). Isopropanol (600 Ml, 3 vol) was charged to the mixture, the mixture was adjusted to 25 °C, and a sample was taken to quantify the amount of dichloromethane remaining (0.25% remained).

Isopropanol (1.9 L, 9.5 vol) was charged to the mixture, followed by dihydroquinine (DHQ, 387 g, 0.908 equiv). The mixture was stirred until a clear solution was formed. Palladium on carbon (10% w/w, KF=63%, 13.7 g) was charged to the mixture. The resulting suspension was degassed with nitrogen twice, then degassed with hydrogen three times. The mixture was stirred for 18-20 hours at 25 °C under 1 atm of hydrogen gas. Analysis of a sample by HPLC showed 0.05% of the third intermediate remaining.

The suspension was filtered and the cake was rinsed with isopropanol (100 Ml, 0.5 vol). Solvent was distilled form the mixture under vacuum at 55 °C to a volume of approximately 1200 Ml (6 vol), then acetonitrile (1.5 L, 7.5 vol) was charged to the mixture to provide a suspension. The distillation and acetonitrile addition were repeated two additional times. A sample of the resultant suspension was diluted with N-methyl pyrrolidone and analyzed to check for residual isopropanol (0.7% found). The suspension was stirred at 80-90 °C for 1-2 hours to provide a clear solution. The mixture was cooled to 5 °C with a cooling rate of 20 °C per hour, then stirred for 2-3 hours. Th suspension was filtered and the cake was washed with cold acetonitrile (400 Ml, 2 vol, at 5 °C). The cake was dried at 55 °C for 18-20 hours without vacuum to provide 591.7 g of 1-13 as a white powder (97.9% AUC purity, 71.8% yield over 4 steps.).

Characterization data for 1-13:

¹H NMR (400 MHz, DMSO) δ 12.50 (s, 1H), 8.75 (d, J=4.5 Hz, 1H), 7.96 (d, J=9.2 Hz, 1H), 7.70-7.48 (m, 3H), 7.41 (dd, J=9.2, 2.5 Hz, 1H), 7.12 (dd, J=11.0, 8.2 Hz, 1H), 7.00 (t, J=7.5 Hz, 1H), 6.97-6.86 (m, 1H), 6.59 (s, 1H), 6.05 (s, 1H), 4.77 (dq, J=12.5, 6.2 Hz, 1H), 3.97 (s, 4H), 3.87-3.66 (m, 2H), 3.57-3.34 (m, 2H), 3.07 (s, 1H), 2.84 (d, J=6.4 Hz, 1H), 1.97 (d, J=36.8 Hz, 3H), 1.87-1.61 (m, 2H), 1.38 (t, J=11.8 Hz, 1H), 1.33-1.18 (m, 2H), 1.16 (d, J=6.6 Hz, 3H), 1.09 (dd, J=6.2, 1.2 Hz, 6H), 0.75 (t, J=7.3 Hz, 3H).

¹³C NMR (101 MHz, DMSO) δ 174.69 (d, J=5.9 Hz), 158.27 (s), 154.97 (s), 152.58 (d, J=6.7 Hz), 147.78 (s), 146.04 (s), 144.19 (s), 142.55-142.06 (m), 131.72 (s), 126.16 (s), 124.38 (d, J=3.4 Hz), 122.73 (s), 122.67-122.04 (m), 119.39 (s), 116.16 (d, J=18.9 Hz), 102.09 (s), 67.71 (s), 66.45 (s), 9.32 (s), 56.87 (s), 55.27 (s), 50.96 (s), 43.12 (s), 35.28 (s), 26.04 (s), 24.89 (s), 21.83 (d, J=2.3 Hz), 21.21 (d, J=5.4 Hz), 17.74 (s), 11.86 (s).

³¹P NMR (162 MHz, DMSO) δ 1.04 (s).

¹⁹F NMR (377 MHz, DMSO) δ _-132.65 (s).

Intermediate 1-12 can be synthesized with different stereochemistry, for example

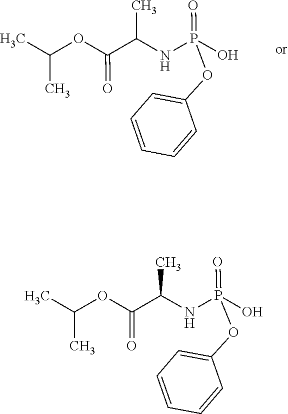

by replacing Intermediate 1-10 with the corresponding amino acid precursor, for example

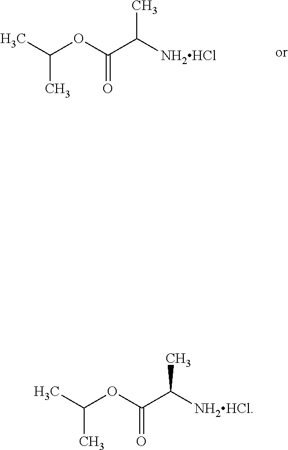

Intermediate 1-13 can be synthesized with different stereochemistry, for example

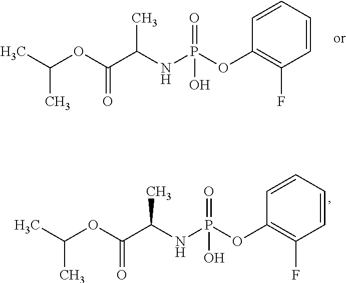 [SMILES: CC(C)OC(=O)C(C)NP(=O)(O)Oc1ccccc1F ; CC(C)OC(=O)[C@@H](C)NP(=O)(O)Oc1ccccc1F]

by replacing Intermediate 1-10 with the corresponding amino acid precursor, for example

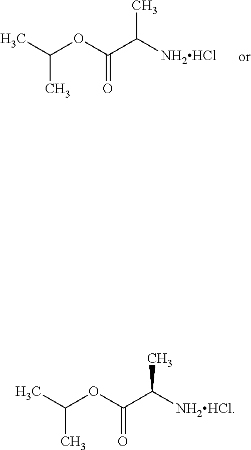

### Example 3. Synthesis of Compound 1

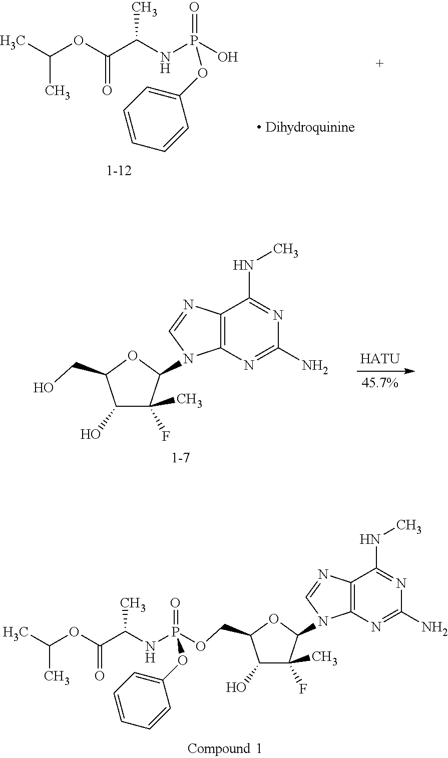

The dihydroquinine salt of isopropyl (hydroxy(phenoxy)phosphoryl)-L-alaninate (1-12, 5.9 g, 1.5 eq.), Compound 1-7 (2.0 g, 1.0 eq), DIPEA (0.83 g, 1.0 eq), and HATU (3.65 g, 1.5 eq) were added into 100 Ml of dichloromethane. The mixture was heated to 40 °C and stirred for 18 hours. The reaction was monitored by TLC and HPLC.

After the reaction was completed, the reaction mixture was cooled to room temperature, washed with 1N hydrochloric acid (100 Ml×2), water (100 Ml×2), and 5% aqueous sodium bicarbonate 15 Ml×1). The separated organic phase was dried with 2 g of anhydrous sodium sulfate, filtered, and concentrated at 40 °C-45 °C under vacuum to give a yellow oil.

Isopropyl acetate (10 Ml of) was added. After stirring, the mixture was concentrated under vacuum. Then, 25 Ml of isopropyl acetate was added. The mixture was heated to 45 °C to afford a clear solution. After stirring at room temperature for 2 hours, the solid precipitate was filtered and dried without vacuum at 45 °C for 15 hours to give 2.0 g of crude Compound 1 (yield: 53.8% mol/mol; HPLC purity: 93.1% by area (containing 3.7% of the compound with the opposite Rp-configuration).

The mixture of crude Compound 1 (2.0 g) and 15 Ml of isopropyl acetate was heated to 80 °C-85 °C to afford a solution. The solution was cooled to 20 °C-25 °C and stirred for 1 hour. The precipitated solid was filtered, washed with isopropyl acetate (1 Ml), and dried without vacuum at 50 °C for 16 hours to give 1.7 g of Compound 1 (yield: 45.7% mol/mol; HPLC purity: 98.99%). ¹H NMR, ¹⁹F NMR, and ³¹P NMR spectra confirmed the structure of Compound 1.

An analogous procedure to the process described above can be used to prepare compounds of alternate stereochemistry by replacing 1-12 with the an intermediate having the desired stereochemistry. For example, these include but are not limited to:

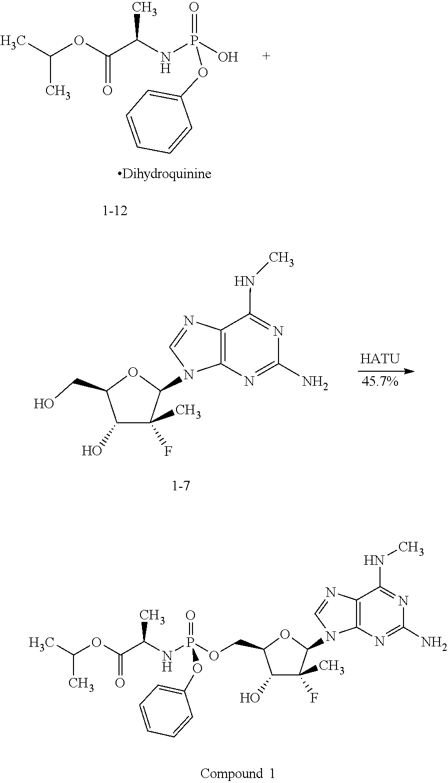

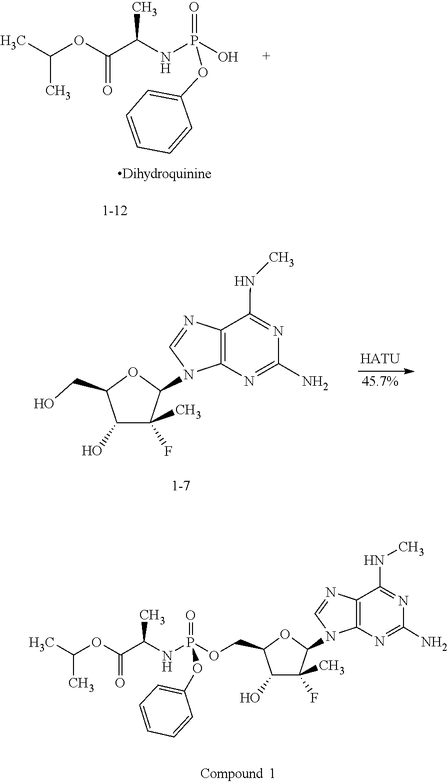

### Example 3B. Synthesis of Isopropyl ((S)-(((2R,3R,4R,5R)-5-(2-amino-6-(methylamino)-9H-purin-9-yl)-4-fluoro-3-hydroxy-4-methyltetrahydrofuran-2-yl)methoxy)(2-fluorophenoxy)phosphoryl)-L-alaninate (1-14)

### Step 1: Coupling of 1-13 and 1-7

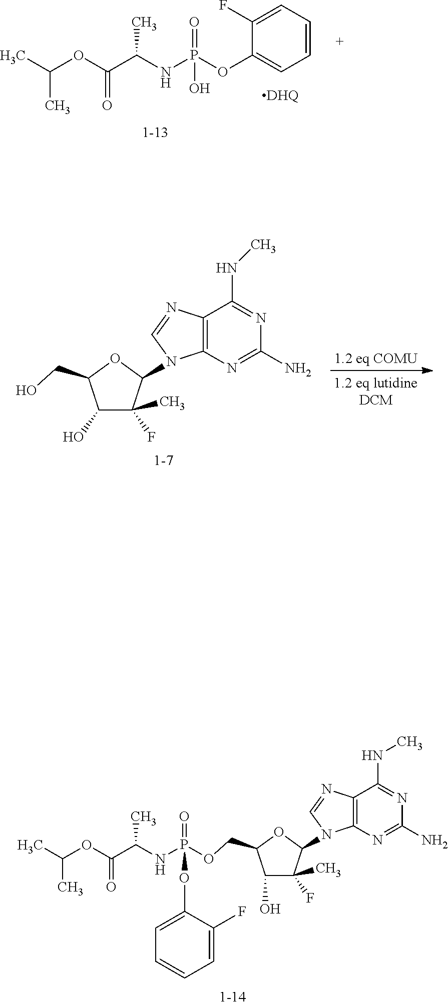

Dichloromethane (1.33 L, 20 vol), Intermediate B (50.0 g, 1.0 equiv, 0.160 mol), dihydroquinine salt of Intermediate A (222 g, 2.2 equiv), and (1-Cyano-2-ethoxy-2-oxoethylidenaminooxy)dimethylamino-morpholino-carbenium hexafluorophosphate (82.3 g, 1.2 equiv) were under nitrogen charged to a 2 L three-neck flask equipped with a reflux condenser and thermometer.

The resulting suspension was heated to 30-35 °C. 2,6-Lutidine (20.6 g, 1.2 equiv) was charged to the reaction mixture over 2 hours and the suspension gradually dissolved during the addition. The mixture was stirred at 30-35 °C for 5-6 hours to provide a clear solution. The reaction was sampled and analysis by HPLC showed less than 5% of Intermediate B remaining. Solution yield calculated by assay of the sample was 64.2 g (66.9% yield). The reaction mixture was cooled to 0-10 °C over 1 hour.

Water (500 Ml, 10 vol) was charged to the mixture and the batch was stirred for 2 minutes. Agitation was halted and the batch was allowed to settle into layers before removal of the aqueous layer. Aqueous hydrochloric acid (20% w/w, 133 g, 4.5 equiv) was charged over a period of 1 hour, over which time dihydroquinine salt precipitated. The solution was filtered, and the filter cake was rinsed with dichloromethane (50 Ml, 1 vol) and the rinse was combined with the filtrate. Aqueous hydrochloric acid (7% w/w, 167 g, 2.0 equiv) was charged to the filtrate and the batch was agitated for 2 min. Agitation was stopped, the batch was allowed to settle into two layers, and the aqueous layer was removed. The organics were washed with aqueous sodium bicarbonate (5% w/w, 500 g, 1.9 equiv) three times, then washed with water (500 Ml, 10 vol). Assay yield of the resultant organic layer was 59.4 g (61.9%). The organic layer was concentrated under vacuum at a temperature of 30-50 °C to a total volume of approximately 200 Ml (4 vol). Toluene (500 Ml, 10 vol) was charged to the mixture and the mixture was concentrated under vacuum at a temperature of 40-55 °C to a total volume of approximately 250 Ml (5 vol). The toluene addition and concentration was repeated one additional time. Ethyl acetate (250 Ml, 5 vol) was charged to the batch, and the mixture was heated to 65-75 °C. While agitating, the mixture was cooled to 5 °C over 3 hours, during which time crystalline solid formed. The batch was held for 4 hours, then filtered to provide a yellow crystalline solid. Theis solid and ethyl acetate (250 Ml, 5 vol) were charged to the flask and heated to 65-75 °C to provide a clear solution. The solution was cooled to 5 °C over 3 hours as crystalline solid formed. The batch was held at 5 °C for 4 hours, then filtered to provide a yellow solid. The yellow solid was dried at 45 °C for 15-16 hours to provide 1-14 (79 g, 56.2% corrected yield) as a yellow crystalline solid.

Characterization data for 1-14:

¹H NMR (400 MHz, DMSO) δ 7.80 (s, 1H), 7.53-7.40 (m, 1H), 7.40-7.26 (m, 2H), 7.25-7.11 (m, 2H), 6.14 (dd, J=12.9, 10.4 Hz, 1H), 6.11-5.91 (m, 3H), 5.78 (d, J=6.4 Hz, 1H), 4.91-4.71 (m, H), 4.58-4.23 (m), 4.15-4.05 (m, 1H), 3.91-3.72 (m, 1H), 2.88 (s, 3H), 1.32-1.20 (m, 3H), 1.17-0.97 (m, 9H).

¹⁹F NMR (377 MHz, DMSO) δ −131.41 (s), −159.74 (s).

³¹P NMR (162 MHz, DMSO) δ 4.42 (s).

MS (ES+): Expected: 600.2; found: 600.3

### Step 2: Aryloxide Displacement

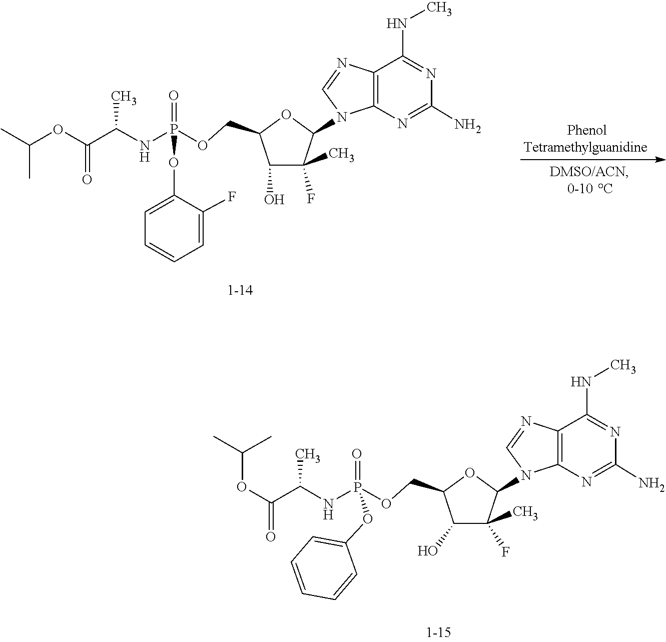

Acetonitrile (255 Ml, 8.5 vol), dimethyl sulfoxide (45 Ml, 1.5 vol), Intermediate C-1 (30 g, 0.05 mol, 1.0 equiv), phenol (94.1 g, 20.0 equiv) 1,1,3,3-tetramethylguanidine (17.3 g, 3.0 equiv) were charged to a 350 Ml flask under nitrogen equipped with a thermometer. The batch was heated to 30 °C for 22 hours while stirring.

The reaction was sampled and remaining Intermediate C-1 was found to be less than 2%. The reaction mixture was cooled to 0-10 °C over 1 hour, and the batch was poured into a reactor containing water (600 Ml, 20 vol) while stirring. Methyl-tert-butyl ether (600 Ml, 20 vol) was charged to the mixture. Aqueous hydrochloric acid (20% w/w, 90 g, 10 equiv) was charged over 0.5 hours to a Ph value of 3-4 while maintaining the temperature of the batch at 0-10 °C. The mixture was stirred for an additional 2 minutes, stirring was halted, the aqueous and organic layers were allowed to separate, and the aqueous layer was collected. Water (300 Ml, 10 vol) was charged to the organic layer, followed by aqueous hydrochloric acid (20% w/w, 32 g, 3.5 equiv) over 0.5 hours while maintaining the temperature of the batch at 0-10 °C.

The mixture was stirred for an additional 2 minutes, stirring was halted, the aqueous and organic layers were allowed to separate, and the aqueous layer was collected. Both aqueous layers were combined and cooled to 0-10 °C. Aqueous sodium bicarbonate (5% w/w, 85 g, 1.0 equiv) was added over a period of 0.5 h to bring the Ph to 8-9, followed by dichloromethane (300 Ml, 10 vol) and water (300 Ml, 10 vol). The mixture was stirred for 2 minutes, allowed to settle into two phases, and the aqueous phase was removed. The resultant organics were concentrated at 30-40 °C under vacuum to an approximate volume of 60 Ml (2 vol).

Acetone (300 Ml, 10 vol) was charged to the mixture. The batch was concentrated at 30-40 °C under vacuum to an approximate volume of 60 Ml (2 vol). Acetone (180 Ml, 6 vol) was charged to the mixture to provide a clear solution, which was cooled to 0-5 °C. Aqueous hydrochloric acid (36.5% w/w, 10 g, 2.0 equiv) was charged in one portion and the mixture was stirred at 0-5 °C for 12 hours. The resultant suspension was filtered, and the recovered solid was dried to provide the bis-hydrochloride salt of 1-15 (19.2 g, 59% yield) as an off-white solid.

### Characterization Data for the Bis-Hydrochloride Salt of 1-15:

¹H NMR (400 MHz, DMSO) δ 9.68 (d, J=4.7 Hz, 1H), 8.14 (s, 3H), 7.36 (t, J=7.9 Hz, 2H), 7.18 (dd, J=17.8, 7.9 Hz, 3H), 6.11 (d, J=18.6 Hz, 2H), 4.84 (hept, J=6.2 Hz, 1H), 4.47 (dd, J=10.4, 5.9 Hz, 1H), 4.43-4.2, 1H), 3.77 (s, 1H), 3.15 (d, J=4.8 Hz, 3H), 1.28-1.07 (m, 12H).

¹³C NMR (101 MHz, DMSO) δ 173.14 (d, J=4.3 Hz), 153.46 (s), 151.11 (d, J=6.0 Hz), 149.58 (s), 130.03 (s), 124.98 (s), 120.62 (d, J=4.6 Hz), 112.08 (s), 102.03 (s), 100.24 (s), 80.59 (s), 72.00 (d, J=18.2 Hz), 68.43 (s), 65.85 (s), 50.42 (s), 40.49 (d, J=13.9 Hz), 40.35 (s), 40.14 (s), 39.93 (s), 39.72 (s), 39.51 (s), 39.30 (s), 31.16 (s), 30.07 (s), 21.84 (d, J=7.2 Hz), 20.08 (d, J=7.2 Hz), 17.15 (s), 16.91 (s).

¹⁹F NMR (377 MHz, DMSO) δ −160.09 (s).

³¹P NMR (162 MHz, DMSO) δ 3.66 (s).

MS (ES+): Expected: 582.22; found: 582.35

### Example 4. Synthesis of Bemnifosbuvir

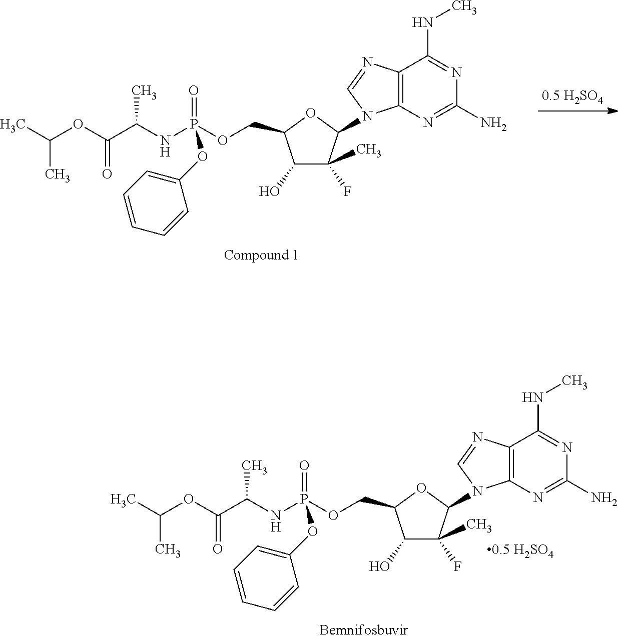 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F ; CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F]

A 250 Ml flask was charged with MeOH (151 Ml) and the solution was cooled to 0-5 °C. A concentrated solution of H₂SO₄ was added dropwise over 10 minutes. A separate flask was charged with Compound 1 (151 g) and acetone (910 Ml), and the H₂SO₄/MeOH solution was added dropwise at 25-30 °C over 2.5 hours. A large amount of solid was precipitated. After the solution was stirred for 12-15 hours at 25-30 °C, the mixture was filtered, washed with MeOH/acetone (25 Ml/150 Ml), and dried at 55-60 °C in vacuum to afford Compound 1-A (121 g, 74%). ¹HNMR: (400 MHz, DMSO-d₆): δ 8.41 (br, 1H), 7.97 (s, 1H), 7.36 (t, J=8.0 Hz, 2H), 7.22 (d, J=8.0 Hz, 2H), 7.17 (t, J=8.0 Hz, 1H), 6.73 (s, 2H), 6.07 (d, J=8.0 Hz, 1H), 6.00 (dd, J=12.0, 8.0 Hz, 1H), 5.81 (br, 1H), 4.84-4.73 (m, 1H), 4.44-4.28 (m, 3H), 4.10 (t, J=8.0 Hz, 2H), 3.85-3.74 (m, 1H), 2.95 (s, 3H), 1.21 (s, J=4.0 Hz, 3H), 1.15-1.10 (m, 9H).

An analogous procedure to the process described above can be used to prepare the hemisulfate salts of compounds of alternate stereochemistry by replacing Compound 1 with the nucleotide phosphoramidate of alternate stereochemistry. For example, these include but are not limited to:

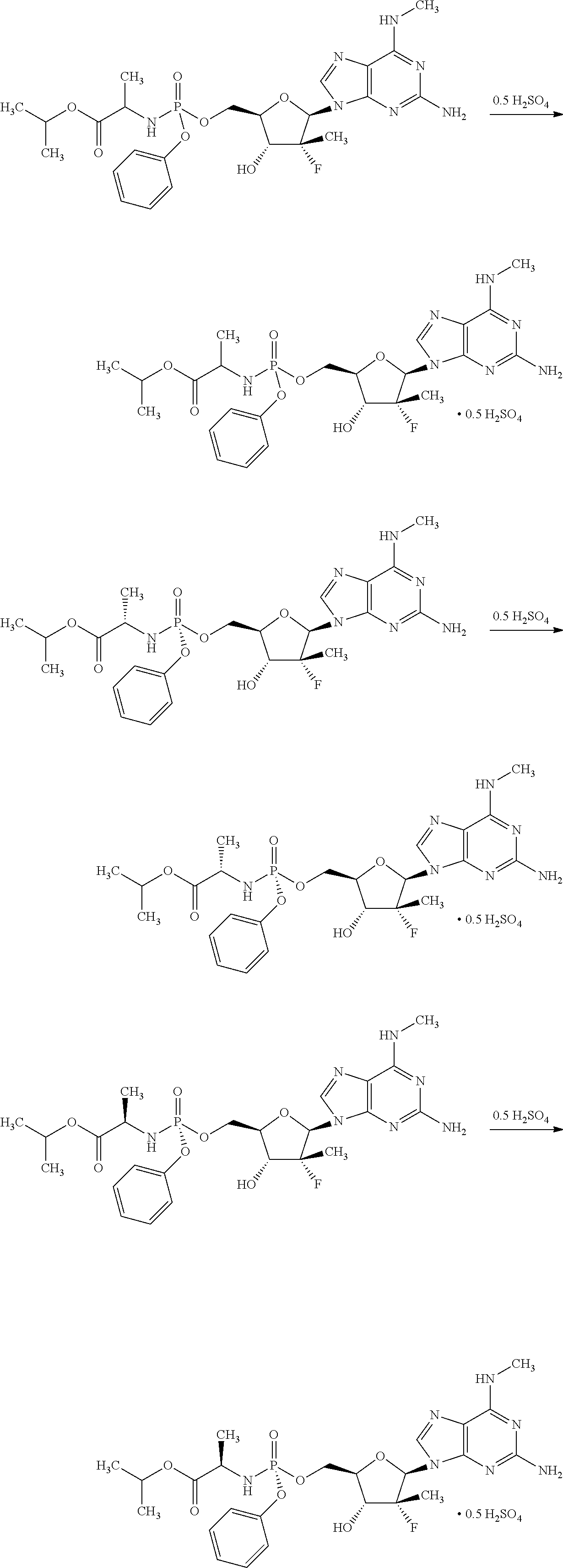

Analytic Method for Bemnifosbuvir: The purity of bemnifosbuvir was obtained using an Agilent 1100 HPLC system with a Waters Xterra Phenyl 5 μm 4.6*250 mm column with the following conditions: 1 Ml/min flow rate, read at 254 nm, 30 °C column temperature, 10 Ml injection volume, and a 30 minute run time. The sample was dissolved in CAN:water (90:10, v/v). The Gradient method for separation is shown below. Rₜ(min) of bemnifosbuvir was approximately 12.0 minutes.

| Time (min) | 0.1% H₃PO₄ in Water (A)% | Acetonitrile (B)% |
|---|---|---|
| 0 | 90 | 10 |
| 20 | 20 | 80 |
| 20.1 | 90 | 10 |
| 30 | 90 | 10 |

### Example 5: Synthesis of Ruzasvir

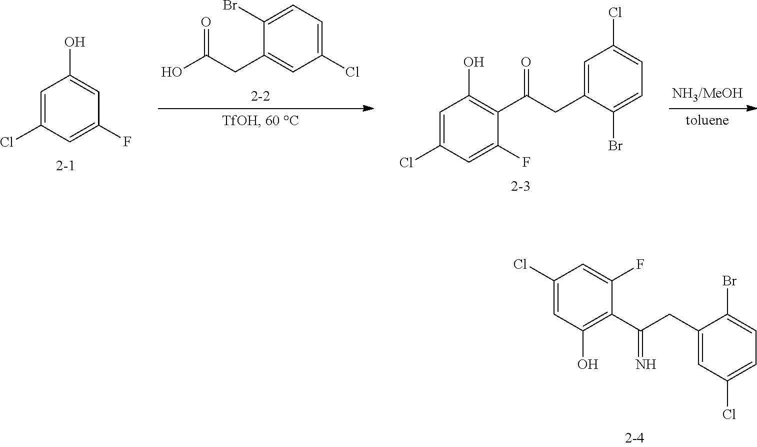 [SMILES: Oc1cc(F)cc(Cl)c1 ; O=C(O)Cc1cc(Cl)ccc1Br ; O=C(Cc1cc(Cl)ccc1Br)c1c(O)cc(Cl)cc1F ; N=C(Cc1cc(Cl)ccc1Br)c1c(O)cc(Cl)cc1F]

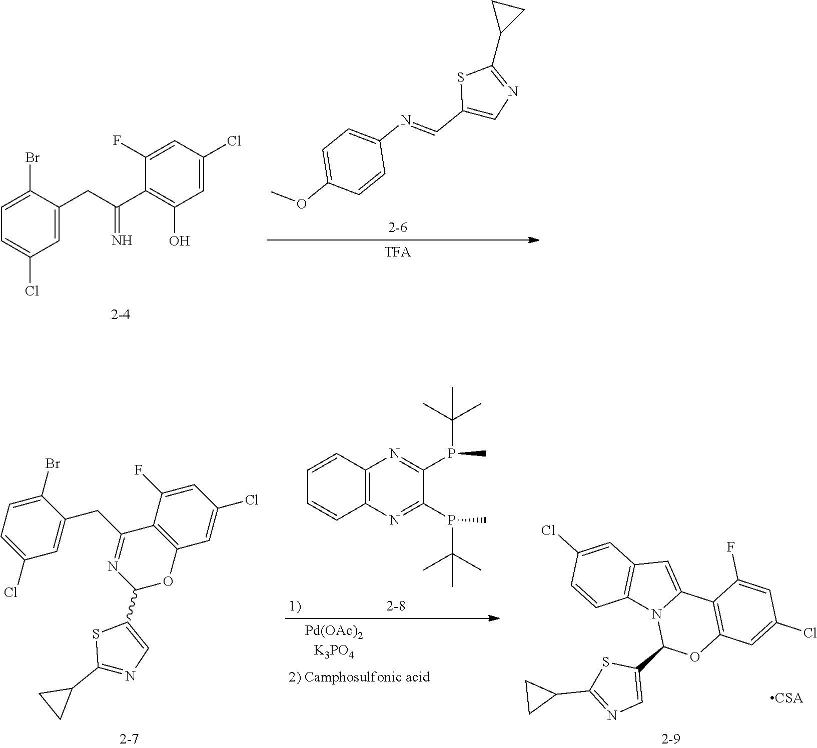 [SMILES: N=C(Cc1cc(Cl)ccc1Br)c1c(O)cc(Cl)cc1F ; COc1ccc(/N=C/c2cnc(C3CC3)s2)cc1 ; Fc1cc(Cl)cc2c1C(Cc1cc(Cl)ccc1Br)=NC(c1cnc(C3CC3)s1)O2 ; C[P@@](c1nc2ccccc2nc1[P@](C)C(C)(C)C)C(C)(C)C ; Fc1cc(Cl)cc2c1-c1cc3cc(Cl)ccc3n1[C@H](c1cnc(C3CC3)s1)O2]

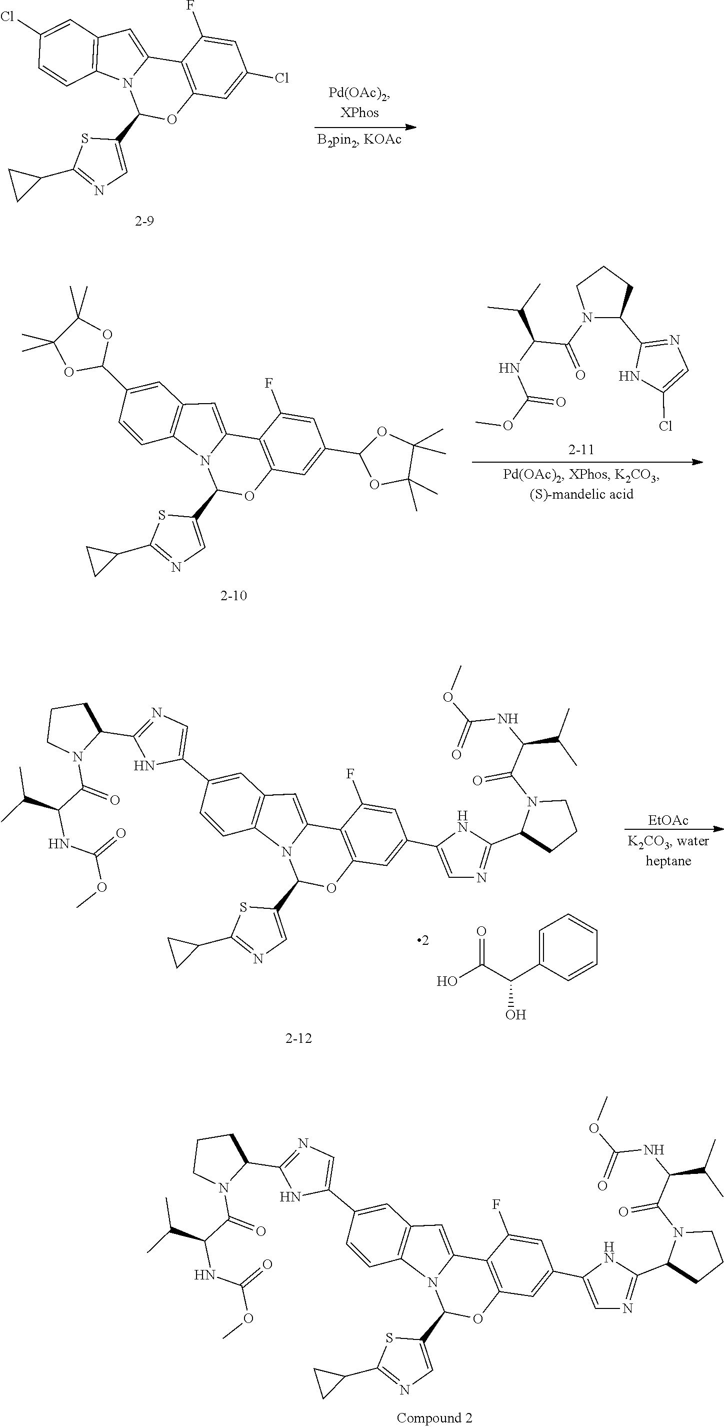

### Synthesis of Compound 2-9

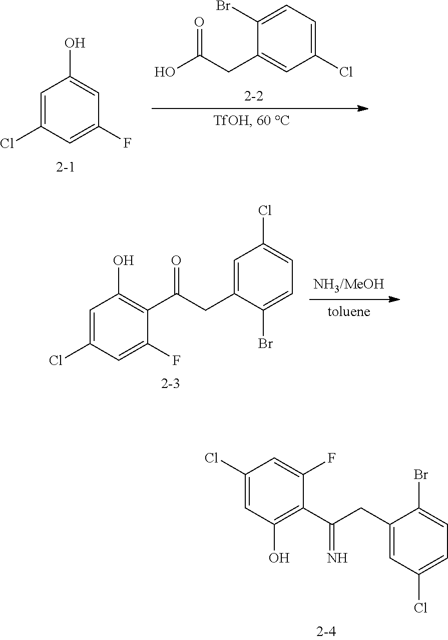 [SMILES: Oc1cc(F)cc(Cl)c1 ; O=C(O)Cc1cc(Cl)ccc1Br ; O=C(Cc1cc(Cl)ccc1Br)c1c(O)cc(Cl)cc1F ; N=C(Cc1cc(Cl)ccc1Br)c1c(O)cc(Cl)cc1F]

To a 250 Ml round bottom flask was added 3-chloro-5-florophenol (Compound 2-1) and 2-(2-bromo-5-chlorophenyl) acetic acid (Compound 2-2 20.02 g, 80.0 mmol) were mixed with TfOH (91 Ml) and heated to 60 °C under an nitrogen atmosphere. After stirring at this temperature for 16 hours, the mixture was cooled to room temperature and poured over a 20 minute period into isopropanol (500 Ml) that had been cooled in an ice/water bath. The resulting slurry was diluted with water (125 Ml), which was added over 10 minutes. After aging for 30 minutes in the ice/water bath, the mixture was filtered, and the collected solid washed with 4:1 isopropanol/water (50 Ml). The solid was dried in vacuo to provide Compound 2-3 (20.14 g, 53.3 mmol, 80% yield).

Compound 2-3 (2.03 g, 5.37 mmol) was taken up in 2-methyltetrahydrofuran (20.3 Ml, 10 volumes), and to this solution was added ammonia in methanol (11.51 Ml of 7N, 81 mmol, 15 eq.). The resulting solution was aged at room temperature for 16 hours, then concentrated by the removal of 25 Ml of solvent and the slurry treated with toluene (70 Ml). The resulting solution was then redistilled to remove a further 35 Ml of solvent and achieve a final solution of the Compound 2-5 in 20 volumes of toluene. This solution was used without further purification.

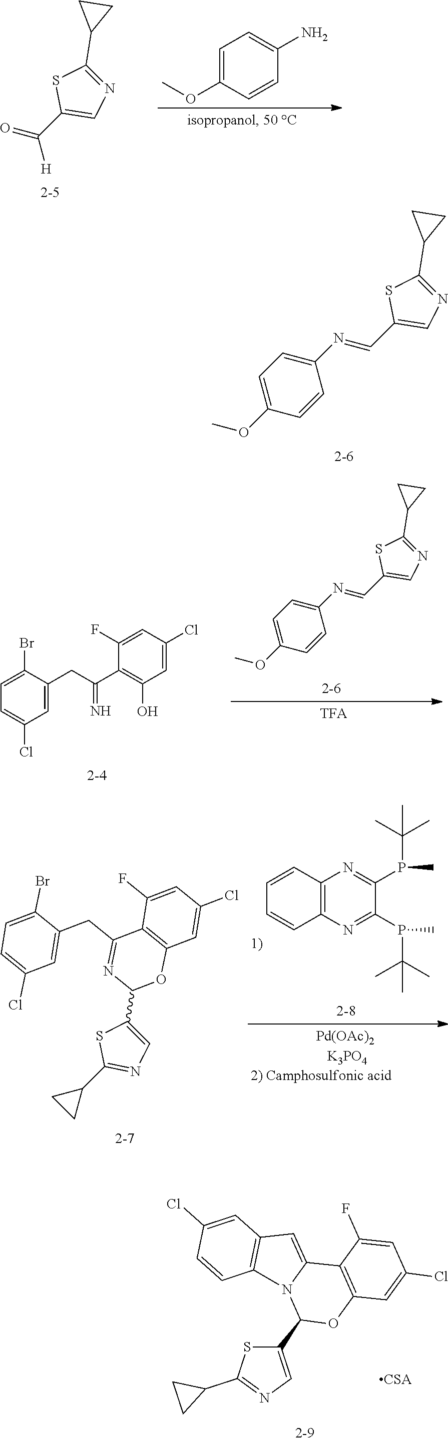

A 1 L round-bottom flask equipped with an air condenser was charged with Compound 2-5 (25 g, 163 mmol), 4-methoxyaniline (22.1 g, 180 mmol), and isopropanol (250 Ml). The resulting slurry was warmed to 50 °C and stirred for 3.5 hours, during which time a precipitate formed. The resulting slurry was cooled to 0 °C, aged for 1 hour, and filtered. The flask and pad were rinsed twice with 0 °C isopropanol (84 Ml), and the solid dried to a constant weight in a vacuum oven at 50 °C to provide Compound 2-6. (39.0 g, 93% yield). ¹H NMR (DMSO-d₆, 400 MHz): δ 8.77 (s, 1H), 8.06 (s, 1H), 7.26 (d, J=8.8 Hz, 2H), 6.96 (d, J=8.8 Hz, 2H), 3.77 (s, 3H), 2.45 (m, 1H), 1.19 (m, 2H), 1.05 (m, 2H)—imine geometry not determined, drawn as CAN for convenience.

A 3-neck roundbottom flask equipped with a magnetic stirbar, a temperature probe, and a nitrogen inlet was charged with Compound 2-6 (7.54 g, 29.2 mmol) and a solution of Compound 2-4 in toluene (5.72 wt %, 174.8 g, 26.5 mmol) was added. The resulting suspension was allowed to stir at room temperature until the solid dissolved, and the resulting solution was cooled using an ice/water bath. TFA (2.45 Ml, 31.8 mmol) was added while the internal temperature was maintained below 5 °C. The resulting solution was allowed to stir in the ice/water bath for 16 hours as it warmed to room temperature. The resulting slurry was filtered, the flask and pad were washed with toluene (27 Ml), and the organic solution washed with aqueous NaHCO₃ (4 wt %, 54 Ml) and water (54 Ml). The organic layer was concentrated in vacuo to ~25 Ml, diluted with isopropanol (110 Ml), and concentrated in vacuo to ~50 Ml total volume. The resulting slurry was warmed to 40 °C, diluted with water (10 Ml, added over 30 minutes), aged at 0 °C for 1 hour, and filtered. The flask and pad were washed with 4:1 isopropanol/water (25 Ml), and the solid dried to a constant weight in a vacuum oven at 50 °C to provide Compound 2-7. (10.1 g, 74% yield)

To a solution of Pd(Oac)₂ (219 mg, 0.98 mmol) and CAN-QuinoxP* (Compound 2-8) (343 mg, 1.03 mmol) in a 100 Ml round bottom flask was added degassed toluene (45 Ml). The solution was subjected to three cycles of evacuation with vacuum and backfill with nitrogen and then purged with nitrogen above surface for 5 minutes. The catalyst solution was then allowed to age at 20 °C for 2 hours. A 1 L, 3-neck round bottom flask fitted with an overhead stirrer was then charged with Compound 2-7 (25 g, 48.8 mmol) and K₃PO₄ (41.4 g, 195 mmol) and toluene (700 Ml). The mixture was subjected to three cycles of vacuum evacuation and nitrogen backfill and then purged with nitrogen above surface for 5 minutes. Degassed water (0.88 Ml, 48.8 mmol) was then added dropwise, after which the premade catalyst solution was added and the resulting reaction heated to 50-55 °C and stirred at this temperature for 11 hours. During the first 6 hours of the reaction time, additional water (5.28 Ml, 293 mmol) was added in six equal portions, each hour. After a total of 11 hours at 50-55 °C, the reaction mixture was cooled to 20 °C and charged with 75 Ml of water and 5 Ml 50% w/v KOH (9N). The aqueous layer was cut away and the organic layer washed with 100 Ml of water. The organic layer was then filtered and concentrated in vacuo and the resulting residue was purified using flash column chromatography to provide Compound 2-9.

The ee was determined using SFC under the following conditions:
Column: ChiralCel OJ-3; 4.6 mm×150 mm; 3 μm particle size
Temperature: 40 °C.
Pressure: 200 bar
Modifier: IPA with 25 Mm isobutyl amine added
Flowrate: 3.0 Ml·min
Conditions: 1% modifier/99% CO₂ to 40% modifier/60% CO₂ over 5 minutes with 1 minute hold at 40% modifier

For purified Compound 2-9: (23 mg, 90% yield, in 91% ee). ¹H NMR (CDCl3, 500 MHz): δ 7.663 (d, J=2.0 Hz, 1H), 7.407 (d, J=0.4 Hz, 1H), 7.200 (dd, J=2.0, 8.8 Hz, 1H), 7.092 (d, J=0.4 8.4 Hz, 1H), 7.048-7.039 (m, 2H), 6.958-6.910 (m, 2H), 2.194-2.153 (m, 1H), 1.275-1.075 (m, 2H), 1.018-0.991 (m, 2H).

The crude product Compound 2-9 was dissolved in about 50 Ml of toluene and 128 Ml of Ipac at 45 °C (S)-Camphorsulfonic acid (10.8 g, 46.4 mmol) was added over 2.5 hours in three portions at 45 °C. It was cooled to room temperature and additional(S)-camphorsulfonic acid (0.57 g, 2.4 mmol) was added. The mixture was aged at room temperature for 16 hrs, then filtered. The solid was washed with 50 ml ½. 5 toluene/isopropyl acetate and then 50 ml isopropyl acetate, and dried with vacuum to afford 27.1 g of Compound 2-9 as the camphorsulfonic acid salt in 96 to >99% ee.

### Synthesis of Compound 2

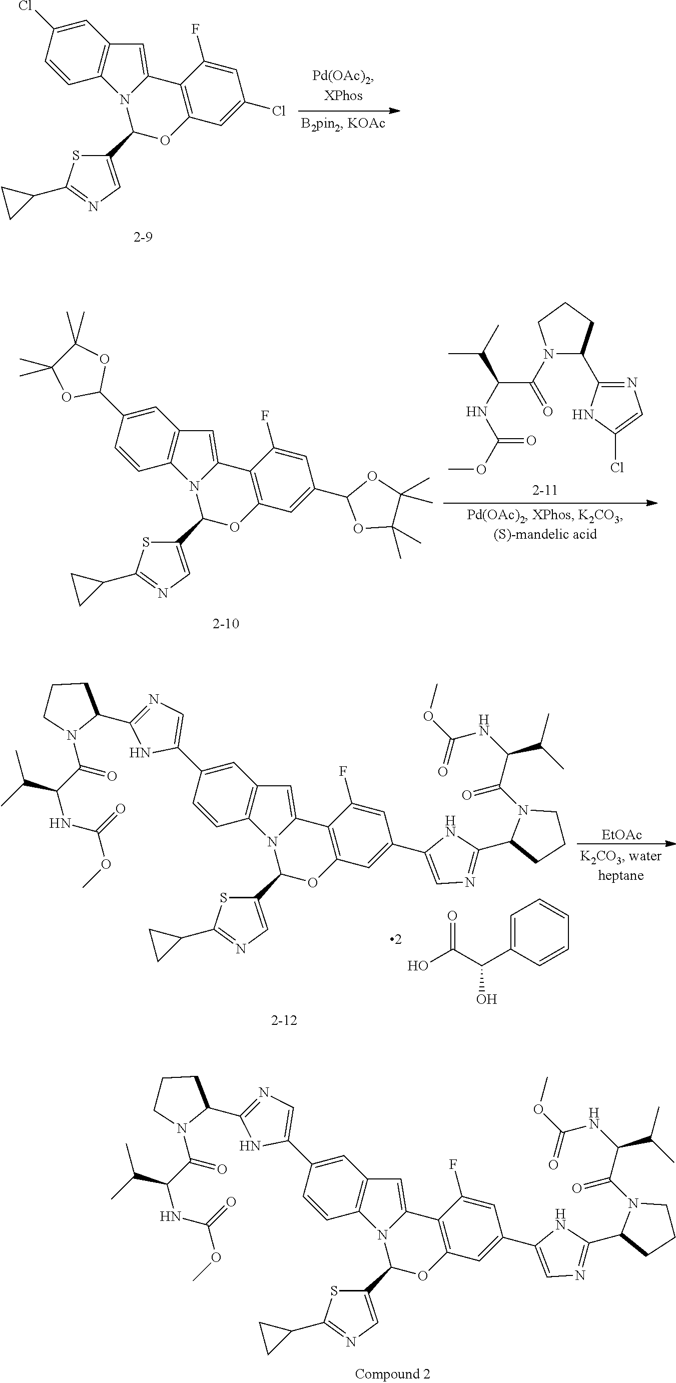

A 500 Ml 3-necked round-bottomed flask with an overhead stirrer was charged with Compound 2-9 ((S)-CSA salt, 10.0 g, or equivalent amount of the free base), bis(pinacolato)diboron (8.50 g), potassium acetate (8.78 g), and 5-chloroindole (0.46 g) under nitrogen. Degassed 2-Me-THF (130 Ml) and water (0.54 Ml) were added. A separate vessel was charged with palladium acetate (0.067 g) and Xphos (2-dicyclohexylphosphino-2′,4′,6′-triisopropylbiphenyl) (0.293 g) and degassed 2-Me-THF (20 Ml) under nitrogen, and the mixture was allowed to stir for 30 minutes then added to the flask Compound 2-9. The mixture was then heated to 75 °C and allowed to age at this temperature for 1 hour or until complete conversion, then allowed to cool to room temperature. Water (30 Ml) was added to the mixture, and the layers separated. The organic layer was washed with 10% brine (30 Ml), then treated with Cuno-3-carbon (1.0 g) for about 15 hours. The mixture was filtered through a Celite-pad to remove carbon. The solution was concentrated in vacuo to about approximately 35 Ml mixture. Seed crystals were added to initiate the crystallization. The mixture was allowed to age at room temperature for 10 minutes before acetonitrile (105 Ml) was added slowly. The resultant slurry was filtered and the collected solid washed with a mixture of acetonitrile/2-Me-THF (3:7, 30 Ml), then dried in a nitrogen stream to provide Compound 2-10. ¹H NMR (CDCl3, 500 MHz), 8.23 (s, 1H), 7.70 (d, J=8.3 Hz, 1H), 7.50 (s, 1H), 7.31 (d, J=9.6 Hz, 1H), 7.29 (d, J=7.1 Hz, 1H), 7.23 (d, J=8.3 Hz, 1H), 7.18 (d, J=3.3 Hz, 1H), 7.10 (s, 1H), 2.15-2.10 (m, 1H), 1.39 (s, 6H), 1.37 (s, 3H), 1.37 (s, 3H), 1.06-1.02 (m, 4H), 1.01-0.95 (m, 4H).

To a high-pressure vessel was charged with compound 2-10 (10.0 g, 16.28 mmol, 1.0 eq), compound 2-11 (11.5 g, 2.15 eq.), 2-Me-THF (90 Ml) and K₂CO₃ (98 Ml, 1 M, 6 eq.). The vessel was degassed. A second reaction vessel was charged with Pd (Oac) 2 (0.11 g, 3%) and Xphos (0.58 g, 7.5%), then degassed followed by addition of degassed 2-Me-THF (20 Ml). The resulting catalyst/ligand slurry was allowed to age for 2 hours at room temperature under nitrogen atmosphere. It was then transferred to the reaction vessel containing Compound 2-11 and rinsed with degassed 2-Me-THF (10 Ml). The resulting reaction mixture was degassed again, and the reaction vessel sealed and heated to 85 to 90 °C for about 8 hours until >99.5% conversion was reached. The reaction was cooled down to room temperature, and the organic layer was sequentially washed with 10% NaCl solution (18 Ml) and 3% NaCl solution (18 Ml). The organic layer was then concentrated in vacuo and azeotropically dried via distillation to provide crude product (14.66 g)

The crude product (14.66 g) in 2-Me-THF (135 Ml) was added to a solution of tri-n-butylphosphine (2.32 Ml) in MeOH (19.4 Ml). The mixture was heated to 70 °C followed by addition of a solution of(S)-mandelic acid (0.94 g) dissolved in 2-Me-THF (3.87 Ml). After aging at 70 °C for several hours, the reaction mixture was cooled to 60 °C and another portion of(S)-mandelic acid (3.06 g) in 2-Me-THF (12.58 Ml) was added. The batch was seeded with the mandelate salt Compound 2-12. A final portion of S-mandelic acid (35.41 g) in 2-Me-THF (22.25 Ml) was charged over 4 hours at 60 °C. The reaction mixture was gradually cooled to 20 °C over 8 hours and aged at 20 °C for 1 hour. The slurry was filtered, and rinsed with 2-Me-THF (containing 2% wt(S)-mandelic acid). The collected solid was dried at 60 °C to provide the bis-mandelate salt Compound 2-12 as a solid (18.74 g)

The bis-mandelate salt Compound 2-12 (6 g) was mixed with ethyl acetate (48 Ml) and water (25.7 Ml). To the biphasic mixture was added 2M potassium carbonate solution (6 Ml, 2.5 equivalents) over 10 minutes, during which a biphasic solution results. The lower aqueous layer was removed, and the organic layer washed sequentially with 8% brine solution (30 Ml) and water (2×30 Ml). The organic layer was azeotropically dried via distillation (final solution volume=30 Ml). Heptane (66 Ml) was charged to an inerted flask. The ethyl acetate stream containing product was added to the heptane over a period of 2 hours. After aging for another 2 hours, the product slurry was filtered, and the wet filter cake washed with a mixture of heptane (10.8 Ml) and EtOAc (2 Ml). The solid was dried in vacuo for about 15 hours at 60 °C to provide ruzasvir as the free base. (4.40 g, MS: M+H 947.4047). 1H NMR (d6-DMSO, 500 MHz) δ (ppm) 8.30 (s, 1H), 8.22 (br s, 1H), 8.10 (br s, 1H), 8.00 (s, 1H), 7.78 (d, J=8.7 Hz, 1H), 7.67-7.65 (m, 2H), 7.52 (br s, 1H), 7.38 (s, 1H), 7.31-7.28 (m, 2H), 7.19 (d, J=3.2 Hz, 1H), 5.16 (t, J=7.4 Hz, 1H), 5.14 (t, J=7.4 Hz, 1H), 4.15-4.11 (m, 2H), 3.91-3.81 (m, 4H), 3.55 (s, 6H), 2.45-2.36 (m, 2H), 2.26 (m, 1H), 2.20-2.13 (m, 2H), 2.13-2.06 (m, 2H), 2.06-2.00 (m, 4H), 0.99 (m, 2H), 0.85-0.77 (m, 14H). 13C NMR (d6-DMSO, 126 MHz) δ (ppm) 175.20, 171.21, 171.15, 158.61 (d, J=251.0 Hz), 156.95, 156.94, 150.05, 149.54 (d, J=7.3 Hz), 148.88, 141.37, 133.88, 133.00, 131.11, 130.68, 129.14, 128.78, 125.35, 121.40, 120.38, 118.15, 117.01, 114.18, 111.04, 110.75, 107.30 (d, J=23.4 Hz), 106.67 (d, J=18.2 Hz), 102.94 (d, J=8.6 Hz), 78.46, 57.95, 57.93, 53.06, 52.91, 51.51, 47.16, 47.11, 31.02, 30.95, 29.08, 24.80, 24.75, 19.35, 19.32, 17.74, 13.88, 11.17, 11.05.

### Example 6. Spray-Dried Dispersions of Ruzasvir

Acetone was dispensed into a solution tank. The mixer in the solution tank was then turned on and ruzasvir was slowly added to the acetone. The mixture was stirred until the solution is clear and free of undissolved solids, approximately 30 minutes. Next, the other excipient (as described in the table below) is slowly added to the solution tank and the solution stirred until free of undissolved solids, approximately 30 minutes.

A spray drying apparatus was set up with a swirl orifice, swirl insert, and spraying systems nozzle body. The drying gas flow rate was set to 80 cfm, the inlet temperature was set to 96 °C, the outlet temperature was set to 40 °C, and the pump set to provide 500 psi of solution feed pressure. The mixture in the solution tank was then processed through the spray dryer and the spray dry processing continued until the mixture in the solution tank was used up. The spray dried material was then placed into drying trays and dried at a temperature of 40 °C for 24 hours.

To prepare ruzasvir in 10% Tween 80, the compound is dissolved in Tween 80 and then the solution of ruzasvir in Tween 80 is added to water.

| | Fasted | | | | Fed | | | |
|---|---|---|---|---|---|---|---|---|
| Formulation | Cmax | Tmax | AUC | SD | Cmax | Tmax | AUC | SD |
| Ruzasvir Neat API | 1415 | 2.4 | 7525 | ±3415 | 655 | 2.4 | 5318 | 1681 |
| Ruzasvir in 10% Tween 80 | 4064 | 3.2 | 18348 | ±7886 | | | | |
| 20% Ruzasvir: 10% TPGS: HPMC E3 | 2636 | 2.8 | 13360 | ±3901 | 1043 | 2.6 | 7144 | 1950 |
| 50% Ruzasvir: HPMC E3 | 2144 | 2.8 | 9550 | ±1659 | | | | |
| 50% Ruzasvir: HPMCAS-M | 2176 | 2.8 | 10279 | ±1833 | | | | |
| 25% Ruzasvir: HPMCAS-L | 1992 | 3.2 | 9147 | ±2689 | | | | |
| 50% Ruzasvir: HPMCAS-L | 2143 | 3.2 | 9367 | ±3420 | 705 | 1.4 | 5657 | 1949 |
| 50% Ruzasvir: HPMCAS-L + SLS | 4084 | 3.2 | 19855 | ±5634 | 1406 | 3.2 | 11436 | 3823 |

### Example 7. Formulation Description and Manufacturing of Exemplary Fixed-Dose Combinations

### Spray-Dried Dispersion

30.8 kg of acetone was dispensed into a 50 L solution tank. Next, 984 g of HPMCAS-L was weighed into a polyethylene bag and 1,000 g of ruzasvir was weighed into a separate polyethylene bag. The mixer in the solution tank was then turned on and the ruzasvir was slowly added to the acetone. The mixture was stirred until the solution is clear and free of undissolved solids, approximately 30 minutes. Next, the HPMCAS-L is slowly added to the solution tank and the solution stirred until free of undissolved solids, approximately 30 minutes.

A spray drying apparatus was set up with a swirl orifice, swirl insert, and spraying systems nozzle body. The drying gas flow rate was set to 80 cfm, the inlet temperature was set to 96 °C, the outlet temperature was set to 40 °C, and the pump set to provide 500 psi of solution feed pressure. The mixture in the solution tank was then flowed at 250 gallons per minute and the spray dry processing continued until the mixture in the solution tank has been used up. The spray dried material was then placed into drying trays and dried at a temperature of 40 °C for 24 hours. The ruzasvir spray dried dispersion was then weighed and stored in heat sealed bags.

### Intragranular Mix

An in-bin hopper equipped with a lid, purge port, vent port and disposable filter was tared. To the tared hopper was added 2.88 kg of the ruzasvir spray dried dispersion, 4.77 kg of bemnifosbuvir, 0.2 kg of colloidal silicon dioxide and a portion of the microcrystalline cellulose (2 scoops). The mixture was blended for approximately 250 revolutions. To this blend was added 2.78 kg of mannitol, the remaining microcrystalline cellulose, 1.5 kg of crospovidone, and 1.5 kg of sodium chloride. The lid of the hopper was secured and the mixture was blended for approximately 200 revolutions. The mixture was slowly added to a conical mill (Quadro Comil® U5, 1750 rpm) and collected in a drum.

A small scoop of the milled mixture was added to sodium stearyl fumarate and shook for 1 minute. The sodium stearyl fumarate mix and the remaining milled mixture was then loaded into a blender and blended for approximately 200 revolutions. The blended material (intragranular mix) was then collected in a drum.

A roller compactor (Gerteis Roller Compactor TG87) was set up with a 1.0 mm square screen, smooth rollers, and a star granulator. The roller compactor was then purged with nitrogen. The intragranular mix was then slowly added into the hopper of the roller compactor and compacted into granules. The granules were collected in a polyethylene lined drum.

### Extragranular Mix

A blender (PDS Globe Pharma Blender (PD139)) was then setup with a 2 cubic foot hopper and nitrogen purge. The extragranular excipients (0.4 kg crospovidone, 0.1 kg colloidal silicon dioxide, and 0.1 kg of sodium stearyl fumarate) were screened through a 0.5 mm (#35 mesh) screen into a double lined drum. Next, the granules of intragranular mix and the screened extragranular excipients were added to the hopper of the blender and the lid was secured. The mixture was blended for approximately 250 revolutions. A small scoop of the blended mixture was added to sodium stearyl fumarate and shook for 1 minute. The sodium stearyl fumarate mix added to the blender containing the granules of intragranular mix and extragranular excipients and the resulting mixture was blended for approximately 200 revolutions. The blend was collected into polyethylene bags.

### Tableting

A tablet press was then configured to press approximately 10 mm×20 mm modified oval tablets. The blend was then hand scooped into the tablet press and any necessary adjustments to produce 1,250 mg tablets were made. The remaining blend was then added to the tablet press and pressed into tablets. The tablets were stored in approximately 10 kg lots in heat seal bags.

| | FDC1A | FDC1B | FDC2A | FDC2B | FDC3 | FDC4A | FDC5A | FDC6 |
|---|---|---|---|---|---|---|---|---|
| | 1000 | 1200 | 1250 | 1250 | 1593 | 1500 | 1500 | 1400 |
| Component | mgW | mgW | mgW | mgW | mgW | mgW | mgW | mgW |
| Ruzasvir | 9 | 8 | 7 | 7 | 6 | 6 | 6 | 6 |
| HPMC E3 | — | — | — | — | 20 | — | — | — |
| HPMCAS-L | — | — | 7 | 7 | — | 6 | — | — |
| Bemnifosbuvir | 30 | 25 | 24 | 24 | 19 | 20 | 20 | 21 |
| TPGS Granulation | — | — | — | — | — | 40 | 40 | — |
| (56.7:28.3:15 | | | | | | | | |
| MCC:Mannitol:TPGS) | | | | | | | | |
| Microcrystalline Cellulose | 26 | 26 | 28 | 27 | 21 | — | — | 33 |
| Mannitol, spray processed | 13 | 13 | 14 | 13 | 10 | 8 | 14 | 16 |
| Crospovidone | 10 | 10 | 10 | 10 | — | — | — | 10 |
| Croscarmellose Sodium | — | — | — | — | 10 | 10 | 10 | — |
| Silicon Dioxide | — | — | 2 | 2 | — | — | — | 2 |
| Colloidal Silicon Dioxide | 2 | 2 | — | — | 1 | 2 | 2 | — |
| Magnesium Stearate | — | — | — | — | 2 | — | — | — |
| Sodium Stearyl Fumarate | 2 | 2 | 2 | 2 | — | 2 | 2 | 2 |
| Fumaric Acid | — | — | — | — | — | — | — | 10 |
| Sodium Chloride | — | 5 | 8 | 8 | 10 | 8 | 8 | — |
| Sodium Lauryl Sulfate | 10 | 10 | — | 2 | — | — | — | — |

| Component | | FDC4A | FDC4B | FDC4C | FDC5A | FDC5B | FDC5C |
|---|---|---|---|---|---|---|---|
| Intragranular | Ruzasvir:HPMCAS-L (1:1) | 12 | 12 | 12 | — | — | — |
| | Ruzasvir | — | — | — | 6 | 6 | 6 |
| | Bemnifosbuvir | 20 | 20 | 20 | 20 | 20 | 20 |
| | TPGS Granulation | 40 | 19 | 27 | 40 | 19 | 27 |
| | Microcrystalline Cellulose | — | 14 | 10 | — | 17 | 13 |
| | Mannitol, spray processed | 8 | 14 | 10 | 14 | 17 | 13 |
| | Croscarmellose Sodium | 6 | 6 | 6 | 6 | 6 | 6 |
| | Colloidal Silicon Dioxide | 1 | 1 | 1 | 1 | 1 | 1 |
| | Sodium Stearyl Fumarate | 1 | 1 | 1 | 1 | 1 | 1 |
| | Sodium Chloride | 8 | 8 | 8 | 8 | 8 | 8 |
| | Percent Intragranular | 95 | 95 | 95 | 95 | 95 | 95 |
| EG | Croscarmellose Sodium | 4 | 4 | 4 | 4 | 4 | 4 |
| | Colloidal Silicon Dioxide | 1 | 1 | 1 | 1 | 1 | 1 |
| | Sodium Stearyl Fumarate | 1 | 1 | 1 | 1 | 1 | 1 |

| | | | | | | |
|---|---|---|---|---|---|---|
| Total | 100 | 100 | 100 | 100 | 100 | 100 |

### Example 8. Dissolution Study

| | |
|---|---|
| Parameter | Setting |

Dissolution Apparatus Parameters

| | |
|---|---|
| Dissolution Apparatus | USP Apparatus II (Paddles) |
| Dissolution Media | 10 Mm Sodium Phosphate, Ph 6.5 + 1% SLS |
| Media Volume | 900 Ml |
| Paddle Speed | 75 rpm, infinity spin 250 rpm |
| Sample Time Points | 10, 15, 30, 45, 60, 75 (infinity spin) |
| Bath Temperature | 37 ± 0.5 °C |
| Filter | QLA 10 micron full flow filter |
| Sinkers | No |

HPLC Method Parameters

| Column | Infinity Lab Poroshell 120 EC-C18, 4.6 | | |
|---|---|---|---|
| | mm × 50 mm, 2.7 μm (PN: 699975-902) | | |
| Flow Rate | 1 Ml/min | | |
| Detection Wavelength | 250 nm | | |
| Column Temperature | 40 °C | | |
| Injection Volume | 10 μL | | |
| Mobile Phase A | 95:5 5 Mm Sodium Phosphate, Ph 7.0: | | |
| | Acetonitrile | | |
| Mobile Phase B | Acetonitrile | | |
| Standard Diluent | 9:1 Methanol:Water | | |
| Needle Wash | 9:1 Methanol:Water | | |
| | Time (min) | % | % |
| Gradient | 0.00 | 60 | 40 |
| | 0.50 | 60 | 40 |
| | 3.00 | 10 | 90 |
| | 3.50 | 10 | 90 |
| | 3.51 | 60 | 40 |
| | 5.00 | 60 | 40 |

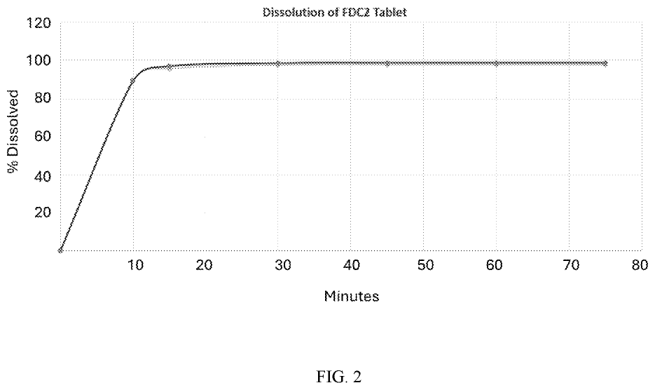

A USP II dissolution apparatus is set to the parameters in the table above. Next, 900 Ml of dissolution media (10 Mm Sodium Phosphate, Ph 6.5+1% SLS) is placed in the apparatus along with one FDC2A tablet. The apparatus is immediately turned on. At the sample time points (10, 15, 30, 45, 60, and 75 minutes) a sample is taken from midway between the surface of the dissolution medium and the top of the rotating blade. After withdrawing each sample, an equivalent volume of 37 C dissolution media is added to maintain a consistent volume. The vessel is covered throughout the test. The samples are analyzed by LC-MS against calibration curves prepared with pure samples of bemnifosbuvir and ruzasvir. The dissolution curve for FDC2A is shown in FIG. 2.

### Example 9. Pharmacokinetic Measurements of Fixed-Dose Combinations

### Dose Administration and Sample Collection:

Tablets were administered by placing the tablet in the back of the dog's mouth and if necessary inducing swallowing. After administration the dog's mouth was inspected to ensure the tablet was swallowed. Each blood collection (about 0.5 Ml per time point) was performed from peripheral vein of each animal into pre-chilled commercial tube containing potassium (K2) EDTA (0.85-1.15 mg) and placed on wet ice until centrifugation.

### Plasma Processing:

Samples were centrifuged (3200 g for 10 minutes at 2 to 8 °C) within one hour of collection. The plasma samples (about 0.2 Ml) were divided into approximate 0.1 Ml×2 aliquots (one for BA, and the other one for back up) and transferred into labeled polypropylene micro-centrifuge tubes. The samples were stored frozen in a freezer set to maintain −60 °C or lower until bio-analysis. Analyte concentrations were measured by LC-MS/MS against a calibration curve.

### Data Analysis

Plasma concentration versus time data will be plotted in graph and analyzed by non-compartmental approaches using the Phoenix WinNonlin 6.3 software program. Related PK parameters will be calculated according to dosing route, e.g. Cₘₐₓ, Tₘₐₓ or % F for extravascular administration, and T_{1/2}, AUC₍₀₋ₜ₎, AUC_{(0-inf)}, MRT₍₀₋ₜ₎, MRT_{(0-inf)} for all routes.

| | Compound 1 Pharmacokinetics | | | | | | |
|---|---|---|---|---|---|---|---|
| | | | | | | | AUC % Rel |
| | | | | | AUC₀₋ₗₐₛₜ | | to Ref. |
| | Cₘₐₓ (ng/Ml) | | Tₘₐₓ (h) | | (ng · h/Ml) | | TAB + CAP |
| | Mean | SD % | Mean | SD % | Mean | SD % | Mean |
| Bemnifosbuvir | 1382 | 88 | 1 | 61 | 1701 | 53 | 100 |
| Tablet + | | | | | | | |
| Ruzasvir | | | | | | | |
| Capsule | | | | | | | |
| FDC1A | 862 | 69 | 1.2 | 37 | 1274 | 58 | 75 |
| FDC2A | 2275 | 81 | 1 | 61 | 2059 | 63 | 121 |
| FDC2B | 1271 | 40 | 0.8 | 34 | 1276 | 26 | 75 |
| FDC3 | 784 | 106 | 2.5 | 104 | 1277 | 71 | 75 |
| FDC4 | 2133 | 40 | 0.7 | 39 | 2600 | 31 | 153 |
| FDC5 | 2667 | 30 | 1.1 | 50 | 3219 | 27 | 189 |
| FDC6 | 1447 | 60 | 0.5 | 0 | 1300 | 39 | 76 |

| | Compound 1-2 Pharmacokinetics | | | | | | |
|---|---|---|---|---|---|---|---|
| | | | | | | | AUC % Rel |
| | | | | | AUC₀₋ₗₐₛₜ | | to Ref. |
| | Cₘₐₓ (ng/Ml) | | Tₘₐₓ (h) | | (ng · h/Ml) | | TAB + CAP |
| | Mean | SD % | Mean | SD % | Mean | SD % | Mean |
| Bemnifosbuvir | 375 | 48 | 1.4 | 39 | 990 | 26 | 100 |
| Tablet + | | | | | | | |
| Ruzasvir | | | | | | | |
| Capsule | | | | | | | |
| FDC1A | 286 | 18 | 1.8 | 25 | 840 | 32 | 85 |
| FDC2A | 511 | 63 | 1.6 | 34 | 1055 | 46 | 106 |
| FDC2B | 374 | 27 | 1.2 | 37 | 856 | 14 | 86 |
| FDC3 | 274 | 54 | 2.8 | 85 | 747 | 25 | 75 |
| FDC4 | 391 | 33 | 1.2 | 37 | 858 | 19 | 87 |
| FDC5 | 440 | 48 | 1.4 | 39 | 1060 | 59 | 107 |
| FDC6 | 343 | 29 | 1.2 | 37 | 790 | 18 | 80 |

| | Compound 1-8 Pharmacokinetics | | | | | | |
|---|---|---|---|---|---|---|---|
| | | | | | | | AUC % Rel |
| | | | | | AUC₀₋ₗₐₛₜ | | to Ref. |
| | Cₘₐₓ (ng/Ml) | | Tₘₐₓ (h) | | (ng · h/Ml) | | TAB + CAP |
| | Mean | SD % | Mean | SD % | Mean | SD % | Mean |
| Bemnifosbuvir | 332 | 66 | 2.4 | 63 | 1054 | 49 | 100 |
| Tablet + | | | | | | | |
| Ruzasvir | | | | | | | |
| Capsule | | | | | | | |
| FDC1A | 182 | 62 | 2 | 61 | 755 | 41 | 72 |
| FDC2A | 282 | 40 | 1.8 | 80 | 809 | 16 | 77 |
| FDC2B | 388 | 71 | 1.8 | 72 | 1276 | 69 | 121 |
| FDC3 | 153 | 68 | 3.4 | 64 | 738 | 52 | 70 |
| FDC4 | 455 | 110 | 1.4 | 39 | 1212 | 87 | 115 |
| FDC5 | 247 | 30 | 1.2 | 37 | 768 | 34 | 73 |
| FDC6 | 472 | 10 | 1.3 | 52 | 1397 | 9 | 133 |

| | Compound 1-7 Pharmacokinetics | | | | | | |
|---|---|---|---|---|---|---|---|
| | | | | | AUC₀₋ₗₐₛₜ | | AUC % Rel |
| | Cₘₐₓ (ng/Ml) | | Tₘₐₓ (h) | | (ng · h/Ml) | | to Ref. |
| | | SD | | SD | | SD | TAB + CAP |
| | Mean | % | Mean | % | Mean | % | Mean |
| Bemnifosbuvir | 1416 | 30 | 4 | 0 | 11155 | 29 | 100 |
| Tablet + | | | | | | | |
| Ruzasvir | | | | | | | |
| Capsule | | | | | | | |
| FDC1A | 882 | 16 | 3.8 | 54 | 7749 | 10 | 69 |
| FDC2A | 1070 | 8.1 | 4.2 | 43 | 11447 | 32 | 103 |
| FDC2B | 1195 | 32 | 3.6 | 25 | 9959 | 16 | 89 |
| FDC3 | 979 | 37 | 4.8 | 63 | 8173 | 18 | 73 |
| FDC4 | 1224 | 26 | 2.8 | 39 | 10175 | 15 | 91 |
| FDC5 | 1114 | 24 | 3.2 | 34 | 8865 | 26 | 79 |
| FDC6 | 1373 | 7 | 3.6 | 25 | 10271 | 14 | 92 |

| | Ruzasvir Pharmacokinetics | | | |
|---|---|---|---|---|
| | | | | AUC % Rel. to |
| | Cmax (ng/Ml) | Tmax (h) | AUC0-last | Reference |
| | (SD %) | (SD %) | (SD %) | TAB + CAP |
| Bemnifosbuvir | 1558 (63) | 3.2 (34) | 6002 (57) | 100 |
| Tablet + | | | | |
| Ruzasvir | | | | |
| Capsule | | | | |
| FDC1 | 474 (56) | 2 (37) | 2095 (53) | 35 |
| FDC2A | 946 (51) | 2.2 (50) | 4597 (58) | 77 |
| FDC2B | 578 (49) | 3 (47) | 2523 (48) | 42 |
| FDC3 | 787 (56) | 3 (75) | 3222 (45) | 54 |
| FDC4 | 1512 (73) | 2.8 (39) | 7641 (74) | 127 |
| FDC5 | 1502 (36) | 2.8 (39) | 7394 (31) | 123 |
| FDC6 | 865 (32) | 2.4 (37) | 3937 (22) | 66 |

### Example 10. Food Effect Study in Dogs

### Pentagastrin Pre-Treatment:

In phase 1, animals were pre-dosed with pentagastrin (0.25 mg/Ml and 0.024 Ml/kg) at 6 μg/kg by intramuscular injection at approximately 30 minutes (±5 min) prior to administration of the combination.

### Famotidine Pre-Treatment:

In phase 2, animals were pretreated with famotidine tablet (40 mg/dog, 2×20 mg tablet/dog) by oral administration at approximately 180 minutes (±10 min) before administration of the combination.

In both phase 1 and phase 2, the tablets were dosed orally as in Example 9.

### Sample Collection

### Blood Collection:

Approximately 0.5 Ml whole blood was collected from animals into commercially available tubes containing Potassium (K2) EDTA as anti-coagulant and 20 μL of 5 Mm dichlorvos solution in water as stabilizer. Each tube was inverted 6-8 times to thoroughly mix the samples and then placed in wet ice before plasma processing.

### Plasma Processing:

Blood samples were centrifuged (3200×g for 10 minutes at 2 to 8 °C) to obtain plasma within one hour of collection. Immediately following centrifugation, 200 Ml of plasma was pipetted into another pre-chilled tube containing 800 Ml 75% MeOH/25% CAN (v/v) with internal standards (100 ng/ml) to precipitate protein.

Each sample was vortex mixed and centrifuged at 12,000 rpm for 15 minutes at approximately 4 °C to separate the supernatant extract and cellular/protein fraction. 400 Ml of the supernatant was transferred into a pre-labeled 1.5 Ml polypropylene tube for bioanalysis. The rest of the supernatant was transferred into another pre-labeled 1.5 Ml polypropylene tube for back up. The samples were maintained at −60 °C or below until LC/MS/MS analysis. Analyte concentrations were measured by LC-MS/MS against a calibration curve.

### Data Analysis

Plasma concentration versus time data was plotted and analyzed by non-compartmental approaches using the Phoenix WinNonlin 6.3 software program. Related PK parameters were calculated and tabulated.

| | | | | | | PHASE 3 - Fed | |
|---|---|---|---|---|---|---|---|
| PHASE 1 - FAST | | | PHASE 2 - FAST | | | (Normal Chow) | |
| Formulation | Treatment | | Formulation | Treatment | | Formulation | Treatment |
| BEM tablet + | pentagastrin | 1 week | BEM tablet + | famotidine | 1 week | BEM tablet + | none |
| RZR capsule | | washout | RZR capsule | | washout | RZR capsule | |
| FDC1A | pentagastrin | | FDC1A | famotidine | | FDC1A | none |
| FDC2A | pentagastrin | | FDC2A | famotidine | | FDC2A | none |
| FDC2B | pentagastrin | | not studied | not studied | | not studied | |
| FDC3 | pentagastrin | | | | | not studied | |
| FDC4 | pentagastrin | | FDC4 | famotidine | | FDC4 | none |
| FDC5 | pentagastrin | | FDC5 | famotidine | | not studied | |
| FDC6 | pentagastrin | | FDC6 | famotidine | | not studied | |

PHASE 1, 2 & 3

| | BEM tablet + | | | | | | | | | | | |
|---|---|---|---|---|---|---|---|---|---|---|---|---|
| | RZR capsule | | FDC1A | | FDC2 | | FDC4 | | FDC5 | | FDC6 | |
| | | Cpd. | | Cpd. | | Cpd. | | Cpd. | | Cpd. | | Cpd. |
| Exposure [AUClast] | RZR | 1-7 | RZR | 1-7 | RZR | 1-7 | RZR | 1-7 | RZR | 1-7 | RZR | 1-7 |
| PHASE 1 | 6002 | 11155 | 2095 | 7749 | 4597 | 11447 | 7641 | 10175 | 7394 | 8865 | 3937 | 10271 |
| [Pentagastrin - FAST] | | | | | | | | | | | | |
| Ratio Rel to TAB + | 100 | 100 | 35 | 69 | 77 | 103 | 127 | 91 | 123 | 79 | 66 | 92 |
| CAP - Phase 1 | | | | | | | | | | | | |
| PHASE 2 | 2172 | 10079 | 1035 | 7396 | 1752 | 11505 | 1696 | 11707 | 650 | 7953 | 2030 | 8860 |
| [Famotidine] | | | | | | | | | | | | |
| Ratio Rel to TAB + | 100 | 100 | 48 | 73 | 81 | 114 | 78 | 116 | 30 | 79 | 93 | 88 |
| CAP - Phase 2 | | | | | | | | | | | | |
| PHASE 3 [FED - Normal | 1167 | 3684 | 694 | 4231 | 1525 | 5177 | 1631 | 4268 | not studied | | not studied | |
| Chow] | | | | | | | | | | | | |
| Ratio Rel to TAB + | 100 | 100 | 59 | 115 | 131 | 141 | 140 | 116 | not studied | | not studied | |
| CAP - Phase 3 | | | | | | | | | | | | |

### Example 11. Preparation of Crystalline Bemnifosbuvir

(Isopropyl ((S)-(((2R,3R,4R,5R)-5-(2-amino-6-(methylamino)-9H-purin-9-yl)-4-fluoro-3-hydroxy-4-methyltetrahydrofuran-2-yl) methoxy) (phenoxy)phosphoryl)-L-alaninate (150 g) was added to acetone (180 Ml) and the mixture was stirred at 20-30 °C to afford a solution. Then, sulfuric acid (12.6 g, 0.5 eq) was slowly added at 15-20 °C and solids gradually precipitated. The mixture was stirred at 15-20 °C for 30 minutes and then stirred at 40-45 °C for 4-5 hours. The mixture was cooled to 25-30 °C and stirred at this temperature for one hour before the mixture was filtered. The resulting cake was rinsed with acetone (150 Ml).

The wet material was dissolved in methanol (150 ml) at 30-40 °C. Acetone (450 ml) was added and then additional acetone was added slowly at 40-45 °C. The mixture was stirred at 40-45 °C for 8-10 hours and then cooled to 25-30 °C. The mixture was filtered, and the resulting cake was rinsed with acetone (150 Ml). The XRPD pattern of the wet bemnifosbuvir Form III is shown in FIG. 3 and the peaks are listed in Table 11A. The peaks in the table correspond to the numbered peaks in FIG. 3.

The wet material was vacuum-dried at 30-35 °C for 4-5 hours and then vacuum-dried at 50-60 °C for around 15 hours to afford dry bemnifosbuvir Form III (130g) in 87% yield. The XRPD pattern of bemnifosbuvir Form III is shown in FIG. 4 and the peaks are listed in Table 111B.

TABLE 11A
Wet Bemnifosbuvir Form III XRPD Peaks

| Peak | | d-spacing | | | | | | |
|---|---|---|---|---|---|---|---|---|
| No. | 2-Theta | (Angstroms) | BG | Height | Height % | Area | Area % | FWHM |
| 1 | 5.159 | 17.1143 | 91 | 521 | 100.0 | 8131 | 100.0 | 0.265 |
| 2 | 6.999 | 12.6199 | 72 | 124 | 23.8 | 3071 | 37.8 | 0.421 |
| 3 | 7.260 | 12.1659 | 71 | 161 | 30.9 | 3991 | 49.1 | 0.421 |
| 4 | 8.860 | 9.9719 | 78 | 220 | 42.4 | 4935 | 60.7 | 0.381 |
| 5 | 9.257 | 9.5455 | 88 | 68 | 13.1 | 796 | 9.8 | 0.199 |
| 6 | 9.898 | 8.9284 | 101 | 61 | 11.7 | 967 | 11.9 | 0.269 |
| 7 | 10.337 | 8.5507 | 82 | 130 | 25.0 | 4312 | 53.0 | 0.564 |
| 8 | 10.860 | 8.1396 | 106 | 106 | 20.3 | 1038 | 12.8 | 0.166 |
| 9 | 12.263 | 7.2119 | 111 | 61 | 11.7 | 1982 | 24.4 | 0.552 |
| 10 | 12.959 | 6.8257 | 138 | 96 | 18.4 | 1506 | 18.5 | 0.267 |
| 11 | 13.580 | 6.5153 | 137 | 163 | 31.3 | 4453 | 54.8 | 0.464 |
| 12 | 13.938 | 6.3484 | 142 | 102 | 19.6 | 2069 | 25.4 | 0.345 |
| 13 | 14.661 | 6.0372 | 159 | 113 | 21.7 | 2527 | 31.1 | 0.380 |
| 14 | 16.258 | 5.4473 | 196 | 70 | 13.4 | 2245 | 27.6 | 0.545 |
| 15 | 16.842 | 5.2597 | 196 | 124 | 23.8 | 2864 | 35.2 | 0.393 |
| 16 | 17.215 | 5.1466 | 191 | 71 | 13.6 | 2286 | 35.5 | 0.691 |
| 17 | 18.138 | 4.8868 | 173 | 101 | 19.4 | 2295 | 28.2 | 0.386 |
| 18 | 19.882 | 4.4619 | 254 | 238 | 45.7 | 5376 | 66.1 | 0.384 |
| 19 | 20.718 | 4.2387 | 282 | 68 | 13.1 | 1304 | 16.0 | 0.326 |
| 20 | 20.999 | 4.2270 | 292 | 66 | 12.7 | 1307 | 16.1 | 0.337 |
| 21 | 21.760 | 4.0809 | 288 | 266 | 51.1 | 6026 | 74.1 | 0.385 |
| 22 | 22.764 | 3.9031 | 258 | 58 | 11.1 | 523 | 6.4 | 0.153 |
| 23 | 24.719 | 3.5988 | 216 | 140 | 26.9 | 3376 | 41.5 | 0.410 |
| 24 | 25.989 | 3.4256 | 191 | 69 | 13.2 | 888 | 10.9 | 0.219 |
| 25 | 31.559 | 2.8326 | 123 | 59 | 11.3 | 801 | 9.9 | 0.231 |

TABLE 11B
Dry Bemnifosbuvir Form III XRPD Peaks

| Peak | | d-spacing | | | | | | |
|---|---|---|---|---|---|---|---|---|
| No. | 2-Theta | (Angstroms) | BG | Height | Height % | Area | Area % | FWHM |
| 1 | 5.180 | 17.0458 | 73 | 517 | 100.0 | 7821 | 100.0 | 0.257 |
| 2 | 6.965 | 12.6814 | 72 | 100 | 19.3 | 2589 | 33.1 | 0.440 |
| 3 | 7.340 | 12.0343 | 72 | 158 | 30.6 | 3452 | 44.1 | 0.371 |
| 4 | 8.921 | 9.9040 | 69 | 229 | 44.3 | 5095 | 65.1 | 0.378 |
| 5 | 9.297 | 9.5049 | 75 | 71 | 13.7 | 1013 | 13.0 | 0.243 |
| 6 | 9.962 | 8.8720 | 92 | 76 | 14.7 | 1355 | 17.3 | 0.303 |
| 7 | 10.360 | 8.5315 | 75 | 131 | 25.3 | 4363 | 55.8 | 0.566 |
| 8 | 10.921 | 8.0944 | 95 | 99 | 19.1 | 1130 | 14.4 | 0.194 |
| 9 | 11.539 | 7.6622 | 88 | 44 | 8.5 | 1380 | 17.6 | 0.533 |
| 10 | 12.259 | 7.2138 | 113 | 61 | 11.8 | 1742 | 22.3 | 0.485 |
| 11 | 12.998 | 6.8057 | 144 | 96 | 18.6 | 1077 | 13.8 | 0.191 |
| 12 | 13.561 | 6.5239 | 127 | 173 | 33.5 | 4822 | 61.7 | 0.474 |
| 13 | 13.901 | 6.3654 | 138 | 90 | 17.4 | 2585 | 33.1 | 0.488 |
| 14 | 14.739 | 6.0053 | 144 | 142 | 27.5 | 2838 | 36.3 | 0.340 |
| 15 | 15.016 | 5.8950 | 150 | 98 | 19.0 | 2844 | 36.4 | 0.493 |
| 16 | 15.614 | 5.6707 | 164 | 80 | 15.5 | 1074 | 13.7 | 0.228 |
| 17 | 16.241 | 5.4531 | 178 | 94 | 18.2 | 2540 | 32.5 | 0.459 |
| 18 | 16.958 | 5.2243 | 173 | 153 | 29.6 | 3419 | 43.7 | 0.380 |
| 19 | 17.281 | 5.1273 | 166 | 92 | 17.8 | 1876 | 24.0 | 0.347 |
| 20 | 18.161 | 4.8806 | 162 | 116 | 22.4 | 2941 | 37.6 | 0.431 |
| 21 | 19.879 | 4.4625 | 249 | 219 | 42.4 | 5302 | 67.8 | 0.412 |
| 22 | 20.678 | 4.2919 | 277 | 69 | 13.3 | 1158 | 14.8 | 0.285 |
| 23 | 21.078 | 4.2114 | 293 | 87 | 16.8 | 1162 | 14.9 | 0.227 |
| 24 | 21.820 | 4.0699 | 282 | 264 | 51.1 | 5395 | 69.0 | 0.347 |
| 25 | 22.829 | 3.8922 | 238 | 86 | 16.6 | 798 | 10.2 | 0.158 |
| 26 | 23.452 | 3.7901 | 233 | 53 | 10.3 | 244 | 3.1 | 0.078 |
| 27 | 24.882 | 3.5755 | 219 | 107 | 20.7 | 2090 | 26.7 | 0.332 |
| 28 | 26.021 | 3.4215 | 184 | 54 | 10.4 | 897 | 11.5 | 0.282 |
| 29 | 31.640 | 2.8255 | 118 | 52 | 10.1 | 864 | 11.0 | 0.282 |

### Example 12. Relative Bioavailability of Fixed-Dose Combination FDC2A

FDC2A (Treatment B) exhibited a high relative bioavailability to the reference formulations (Treatment A): ranging from approximately 7800 to 114% based on the Cₘₐₓ and from 87% to 113% (based on the AUC_{0-inf.} Of Compound 1, Compound 1-7, and RZR.
Treatment-A: BEM tablets (Reference-1) and RZR capsules (Reference-2) taken concomitantly under fasted conditions;
Treatment-B: FDC2A (Test) administered under fasted conditions;
Treatment-C: FDC2A (Test) administer under fed conditions (HFHC meal);
Treatment-D: BEM tablets (Reference-1) and RZR capsules (Reference-2) taken concomitantly under fed conditions (HFHC meal)

Data are presented as arithmetic mean (arithmetic CV %) except for Tₘₐₓ, which is presented as median (minimum-maximum). Abbreviation: NC: Not Calculated.

TABLE 12A
Mean (CV %) Plasma Compound 1 Pharmacokinetic Parameter Estimates
Following a Single-Dose Oral Administration of BEM/RZR

| | Treatment-A | Treatment-B | Treatment-C | Treatment-D |
|---|---|---|---|---|
| | (Ref, Fasted) | (FDC2A, Fasted) | (FDC2A, Fed) | (Ref, Fed) |
| | N = 22 | N = 24 | N = 24 | N = 12 |
| Cₘₐₓ (ng/Ml) | 4183.42 (36.6) | 3300.16 (36.2) | 1933.24 (69.3) | 1883.97 (41.4) |
| Tₘₐₓ (h) | 0.50 (0.25-1.00) | 0.50 (0.25-1.00) | 2.00 (0.75-6.00) | 1.75 (0.78-4.00) |
| AUC₀₋ₜ (h · ng/Ml) | 3722.05 (40.4) | 3226.96 (45.7) | 3543.74 (48.2) | 3881.82 (51.2) |
| AUC_{0-∞} (h · ng/Ml) | 3726.20 (40.4) | 3245.13 (46.4)a | 3483.33 (45.5)c | 4072.45 (48.4)d |
| T_{half} (h) | 0.96 (23.0) | 0.90 (19.3)a | 0.72 (27.6)c | 0.79 (25.9)d |
| T_{lag} (h) | 0.00 (NC) | 0.00 (NC) | 0.04 (228.4) | 0.02 (346.4) |

aN = 23;
^{b} N = 16;
cN = 21;
dN = 11;
^{e} N = 20

TABLE 12B
Mean (CV %) Plasma Compound 1-2 Pharmacokinetic Parameter Estimates
Following a Single-Dose Oral Administration of BEM/RZR

| | Treatment-A | Treatment-B | Treatment-C | Treatment-D |
|---|---|---|---|---|
| | (Ref, Fasted) | (FDCA2, Fasted) | (FDC2A, Fed) | (Ref, Fed) |
| | N = 22 | N = 24 | N = 24 | N = 12 |
| Cₘₐₓ (ng/Ml) | 778.10 (43.8) | 796.49 (56.9) | 528.00 (44.0) | 458.00 (32.4) |
| Tₘₐₓ (h) | 1.00 (0.75-1.50) | 0.88 (0.50-4.00) | 3.00 (1.50-8.18) | 3.00 (2.00-4.02) |
| AUC₀₋ₜ (h · ng/Ml) | 2438.98 (40.1) | 2335.29 (27.6) | 2211.50 (38.6) | 2060.02 (36.7) |
| AUC_{0-∞} (h · ng/Ml) | 2467.20 (39.1) | 2354.91 (27.3) | 2253.88 (38.1)b | 2072.07 (36.2) |
| T_{half} (h) | 2.54 (21.8) | 2.57 (21.8) | 2.45 (24.0)b | 2.65 (9.8) |
| T_{lag} (h) | 0.02 (323.7) | 0.04 (228.5) | 0.31 (71.3)b | 0.23 (56.2) |

^{a} N = 19;
bN = 23;
^{c} N = 20

TABLE 12C
Mean (CV %) Plasma Compound 1-8 Pharmacokinetic Parameter Estimates
Following a Single-Dose Oral Administration of BEM/RZR

| | Treatment-A | Treatment-B | Treatment-C | Treatment-D |
|---|---|---|---|---|
| | (Ref, Fasted) | (FDC2A, Fasted) | (FCD2A, Fed) | (Ref, Fed) |
| | N = 22 | N = 24 | N = 24 | N = 12 |
| Cₘₐₓ (ng/Ml) | 856.76 (27.7) | 726.39 (37.6) | 385.26 (29.0) | 465.52 (17.1) |
| Tₘₐₓ (h) | 1.50 (0.75-3.00) | 1.50 (0.50-4.02) | 3.50 (2.00-24.00) | 3.50 (2.00-4.02) |
| AUC₀₋ₜ (h · ng/Ml) | 5416.23 (19.4) | 4899.42 (29.6) | 3470.83 (25.5) | 3883.49 (16.4) |
| AUC_{0-∞} (h · ng/Ml) | 5577.59 (18.8)a | 4984.22 (29.4) | 3594.74 (26.2) | 4021.67 (15.9) |
| T_{half} (h) | 16.84 (80.1)a | 15.66 (58.0) | 20.29 (98.2) | 21.73 (84.7) |
| T_{lag} (h) | 0.07 (167.1) | 0.12 (128.2) | 0.55 (39.5) | 0.31 (60.3) |

aN = 21;
^{b} N = 17

TABLE 12D
Mean (CV %) Plasma Compound 1-7 Pharmacokinetic Parameter Estimates
Following a Single-Dose Oral Administration of BEM/RZR

| | Treatment-A | Treatment-B | Treatment-C | Treatment-D |
|---|---|---|---|---|
| | (Ref, Fasted) | (FDC2A, Fasted) | (FDC2A, Fed) | (Ref, Fed) |
| | N = 22 | N = 24 | N = 24 | N = 12 |
| Cₘₐₓ (ng/Ml) | 243.00 | 226.02 | 252.75 | 283.15 |
| | (25.5) | (22.9) | (22.5) | (22.1) |
| Tₘₐₓ (h) | 4.00 | 4.00 | 6.00 | 6.00 |
| | (2.50-8.00) | (2.50-8.00) | (4.00-8.18) | (6.00-6.03) |
| AUC₀₋ₜ | 3733.62 | 3493.81 | 3829.69 | 4482.55 |
| (h · ng/Ml) | (30.7) | (27.6) | (22.5) | (20.6) |
| AUC_{0-∞} | 3985.18 | 3576.82 | 3928.72 | 4564.79 |
| (h · ng/Ml) | (24.1)a | (27.3)b | (22.5) | (19.8) |
| T_{half} (h) | 25.17 | 21.95 | 24.27 | 22.47 |
| | (70.7)a | (61.5)b | (89.1) | (49.0) |
| T_{lag} (h) | 0.26 | 0.29 | 0.82 | 0.34 |
| | (55.0) | (32.6) | (31.7) | (97.0) |
| C₂₄ (ng/Ml) | 43.24 | 37.66 | 47.09 | 52.53 |
| | (34.3)a | (37.7) | (31.9) | (34.9) |

aN = 21;
bN = 23;
^{c} N = 17;
^{d} N = 19

TABLE 12E
Mean (CV %) Plasma RZR Pharmacokinetic Parameter Estimates
Following a Single-Dose Oral Administration of BEM/RZR

| | Treatment-C | Treatment-D | Treatment-E | Treatment-F |
|---|---|---|---|---|
| | (Ref, Fasted) | (FDC2A, Fasted) | (FDC2A, Fed) | (Ref, Fed) |
| | N = 22 | N = 24 | N = 24 | N = 12 |
| Cₘₐₓ (ng/Ml) | 179.80 | 192.59 | 206.54 | 200.57 |
| | (50.2) | (41.5) | (34.5) | (28.1) |
| Tₘₐₓ (h) | 2.50 | 3.50 | 4.00 | 5.42 |
| | (1.50-4.00) | (2.00-4.02) | (2.00-12.00) | (46.2) |
| AUC₀₋ₜ | 2510.24 | 2809.67 | 3811.73 | 4004.45 |
| (h · ng/Ml) | (52.8) | (43.1) | (43.5) | (46.1) |
| AUC_{0-∞} | 2688.21 | 2899.92 | 3942.50 | 4195.83 |
| (h · ng/Ml) | (49.0)a | (43.0) | (44.8) | (47.4) |
| T_{half} (h) | 31.51 | 30.60 | 32.15 | 36.12 |
| | (19.5)a | (10.0) | (17.9) | (10.4) |
| T_{lag} (h) | 0.30 | 0.14 | 0.77 | 0.17 |
| | (42.4) | (108.5) | (75.9) | (346.4) |
| C₂₄ (ng/Ml) | 32.85 | 34.29 | 52.72 | 53.29 |
| | (52.2)a | (46.2) | (46.3) | (50.5) |

aN = 21;
^{b} N = 18;
^{c} N = 19

### Example 13. Relative Bioavailability of Fixed-Dose Combination Under Fasted Conditions

This study was performed to determine the pharmacokinetic properties of the fixed dose combination under fasted conditions, with or without famotidine. The study conditions are described below.
Treatment-A: BEM tablets (Reference-1) and RZR capsules (Reference-2) taken concomitantly under fasted conditions;
Treatment-B: FDC2A (Test) administered under fasted conditions;
Treatment-C: Famotidine administered the evening prior to dosing (approximately 1 hour before dinner) and approximately 12 hours later. BEM tablets (Reference-1) and RZR capsules (Reference-2) were administered concomitantly under fasted conditions approximately 2 hours after the second dose of famotidine);
Treatment-D: Famotidine administered the evening prior to dosing (approximately 1 hour before dinner) and approximately 12 hours later. FDC2A (Test) was administered under fasted conditions simultaneously with the second dose of famotidine;
Treatment-E: Famotidine administered the evening prior to dosing (approximately 1 hour before dinner) and approximately 12 hours later. FDC2A (Test) was administered under fasted conditions approximately 2 hours after the second dose of famotidine

TABLE 13A
Mean (CV %) Plasma Compound 1 Pharmacokinetic Parameter Estimates Following
a Single-Dose Oral Administration of BEM/RZR under Fasted Conditions

| | | | Treatment-C | Treatment-D | Treatment-E |
|---|---|---|---|---|---|
| | Treatment-A | Treatment-B | (Ref + | (FDC2A + | (FDC2A 2 h + |
| | (Ref) | (FDC2A) | Famotidine) | Famotidine) | Famotidine) |
| | N = 22 | N = 24 | N = 12 | N = 12 | N = 12 |
| Cₘₐₓ (ng/Ml) | 4183.42 | 3300.16 | 3777.97 | 3491.33 | 2506.89 |
| | (36.6) | (36.2) | (31.6) | (35.3) | (29.5) |
| Tₘₐₓ (h) | 0.50 | 0.50 | 0.50 | 0.50 | 0.50 |
| | (0.25-1.00) | (0.25-1.00) | (0.25-0.75) | (0.50-1.00) | (0.25-1.50) |
| AUC₀₋ₜ | 3722.05 | 3226.96 | 2852.07 | 3516.81 | 2188.99 |
| (h · ng/Ml) | (40.4) | (45.7) | (35.9) | (37.4) | (41.9) |
| AUC_{0-∞} | 3726.20 | 3245.13 | 2856.23 | 3521.95 | 2191.88 |
| (h · ng/Ml) | (40.4) | (46.4)^{a} | (35.9) | (37.3) | (41.9) |
| T_{half} (h) | 0.96 | 0.90 | 0.88 | 1.15 | 0.89 |
| | (23.0) | (19.3)^{a} | (21.1) | (34.9) | (16.2) |
| T_{lag} (h) | 0.00 | 0.00 | 0.00 | 0.00 | 0.00 |
| | (NC) | (NC) | (NC) | (NC) | (NC) |

^{a}N = 23

TABLE 13B
Mean (CV %) Plasma Compound 1-2 Pharmacokinetic Parameter Estimates Following
a Single-Dose Oral Administration of BEM/RZR under Fasted Conditions

| | | | Treatment-C | Treatment-D | Treatment-E |
|---|---|---|---|---|---|
| | Treatment-A | Treatment-B | (Ref + | (FDC2A + | (FDC2A 2 h + |
| | (Ref) | (FDC2A) | Famotidine) | Famotidine) | Famotidine) |
| | N = 22 | N = 24 | N = 12 | N = 12 | N = 12 |
| Cₘₐₓ (ng/Ml) | 778.10 | 796.49 | 753.69 | 610.80 | 488.33 |
| | (43.8) | (56.9) | (20.4) | (27.5) | (28.5) |
| Tₘₐₓ (h) | 1.00 | 0.88 | 1.00 | 1.00 | 0.75 |
| | (0.75-1.50) | (0.50-4.00) | (0.50-0.75) | (0.75-2.00) | (0.50-3.00) |
| AUC₀₋ₜ | 2438.98 | 2335.29 | 2117.72 | 2145.12 | 1555.68 |
| (h · ng/Ml) | (40.1) | (27.6) | (15.6) | (28.7) | (25.5) |
| AUC_{0-∞} | 2467.20 | 2354.91 | 2145.73 | 2163.54 | 1579.75 |
| (h · ng/Ml) | (39.1) | (27.3) | (15.4) | (28.2) | (24.3) |
| T_{half} (h) | 2.54 | 2.57 | 2.41 | 2.75 | 2.85 |
| | (21.8) | (21.8) | (27.7) | (19.9) | (41.4) |
| T_{lag} (h) | 0.02 | 0.04 | 0.04 | 0.04 | 0.04 |
| | (323.7) | (228.5) | (233.5) | (233.5) | (233.5) |

TABLE 13C
Mean (CV %) Plasma Compound 1-7 Pharmacokinetic Parameter Estimates Following
a Single-Dose Oral Administration of BEM/RZR under Fasted Conditions

| | | | Treatment-C | Treatment-D | Treatment-E |
|---|---|---|---|---|---|
| | Treatment-A | Treatment-B | (Ref + | (FDC2A + | (FDC2A 2 h + |
| | (Ref) | (FDC2A) | Famotidine) | Famotidine) | Famotidine) |
| | N = 22 | N = 24 | N = 12 | N = 12 | N = 12 |
| Cₘₐₓ (ng/Ml) | 243.00 | 226.02 | 222.41 | 237.20 | 193.22 |
| | (25.5) | (22.9) | (21.8) | (24.5) | (30.8) |
| Tₘₐₓ (h) | 4.00 | 4.00 | 5.13 | 6.00 | 4.00 |
| | (2.50-8.00) | (2.50-8.00) | (2.50-8.03) | (3.00-6.00) | (3.00-8.00) |
| AUC₀₋ₜ | 3733.62 | 3493.81 | 3468.03 | 3878.15 | 3211.68 |
| (h · ng/Ml) | (30.7) | (27.6) | (18.5) | (19.5) | (27.7) |
| AUC_{0-∞} | 3985.18 | 3576.82 | 3519.98 | 3919.28 | 3286.84 |
| (h · ng/Ml) | (24.1)^{a} | (27.3)^{b} | (18.4) | (19.8)^{c} | (28.4) |
| T_{half} (h) | 25.17 | 21.95 | 17.08 | 19.18 | 19.84 |
| | (70.7)^{a} | (61.5)^{b} | (33.3) | (54.3)^{c} | (59.2) |
| T_{lag} (h) | 0.26 | 0.29 | 0.23 | 0.17 | 0.27 |
| | (55.0) | (32.6) | (56.2) | (97.7) | (61.7) |
| C₂₄ (ng/Ml) | 43.24 | 37.66 | 36.95 | 43.30 | 38.48 |
| | (34.3)^{a} | (37.7) | (23.1) | (30.3) | (31.9) |

^{a}N = 21;
^{b}N = 23;
^{c}N = 11

TABLE 13D
Mean (CV %) Plasma Compound 1-8 Pharmacokinetic Parameter Estimates Following
a Single-Dose Oral Administration of BEM/RZR under Fasted Conditions

| | | | Treatment-C | Treatment-D | Treatment-E |
|---|---|---|---|---|---|
| | Treatment-A | Treatment-B | (Ref + | (FDC2A + | (FDC2A 2 h + |
| | (Ref) | (FDC2A) | Famotidine) | Famotidine) | Famotidine) |
| | N = 22 | N = 24 | N = 12 | N = 12 | N = 12 |
| Cₘₐₓ (ng/Ml) | 856.76 | 726.39 | 1099.51 | 618.99 | 667.92 |
| | (27.7) | (37.6) | (43.2) | (35.1) | (37.4) |
| Tₘₐₓ (h) | 1.50 | 1.50 | 1.50 | 1.50 | 1.58 |
| | (0.75-3.00) | (0.50-4.02) | (1.00-3.00) | (1.00-3.00) | (0.75-4.00) |
| AUC₀₋ₜ | 5416.23 | 4899.42 | 6466.22 | 4972.27 | 5492.13 |
| (h · ng/Ml) | (19.4) | (29.6) | (22.2) | (26.0) | (26.8) |
| AUC_{0-∞} | 5577.59 | 4984.22 | 6533.56 | 5148.33 | 5570.74 |
| (h · ng/Ml) | (18.8)^{a} | (29.4) | (21.9) | (26.1) | (26.2) |
| T_{half} (h) | 16.84 | 15.66 | 13.90 | 20.11 | 14.53 |
| | (80.1)^{a} | (58.0) | (45.2) | (76.4) | (51.2) |
| T_{lag} (h) | 0.07 | 0.12 | 0.08 | 0.17 | 0.10 |
| | (167.1) | (128.2) | (147.7) | (73.9) | (123.6) |

^{a}N = 21

TABLE 13E
Mean (CV %) Plasma Ruzasvir Pharmacokinetic Parameter Estimates Following
a Single-Dose Oral Administration of BEM/RZR under Fasted Conditions

| | | | Treatment-C | Treatment-D | Treatment-E |
|---|---|---|---|---|---|
| | Treatment-A | Treatment-B | (Ref + | (FDC2A + | (FDC2A 2 h + |
| | (Ref) | (FDC2A) | Famotidine) | Famotidine) | Famotidine) |
| | N = 22 | N = 24 | N = 12 | N = 12 | N = 12 |
| Cₘₐₓ (ng/Ml) | 179.80 | 192.59 | 94.36 | 190.81 | 104.06 |
| | (50.2) | (41.5) | (34.7) | (43.4) | (65.4) |
| Tₘₐₓ (h) | 2.50 | 3.50 | 3.50 | 4.00 | 4.00 |
| | (1.50-4.00) | (2.00-4.02) | (1.50-6.00) | (2.50-4.00) | (2.00-6.00) |
| AUC₀₋ₜ | 2510.24 | 2809.67 | 1572.73 | 3156.21 | 1735.74 |
| (h · ng/Ml) | (52.8) | (43.1) | (51.6) | (49.9) | (68.7) |
| AUC_{0-∞} | 2688.21 | 2899.92 | 1648.84 | 3284.49 | 1812.84 |
| (h · ng/Ml) | (49.0)^{a} | (43.0) | (49.9) | (50.6) | (67.2) |
| T_{half} (h) | 31.51 | 30.60 | 34.34 | 34.95 | 32.11 |
| | (19.5)^{a} | (10.0) | (14.8) | (15.1) | (22.2) |
| T_{lag} (h) | 0.30 | 0.14 | 0.00 | 0.00 | 0.06 |
| | (42.4) | (108.5) | (NC) | (NC) | (180.9) |
| C₂₄ (ng/Ml) | 32.85 | 34.29 | 20.30 | 36.00 | 22.08 |
| | (52.2)^{a} | (46.2) | (53.3) | (50.0) | (71.8) |

Abbreviation: NC: Not Calculated
^{a}N = 21

### Example 14. Relative Bioavailability of Fixed-Dose Combination

This was a 2-part, adaptive study to mainly assess, under fasting conditions, the relative bioavailability (Rba) of prototype FDC tablets (FDC2A, treatment B), each containing BEM 275 mg/RZR 90 mg, using co-administered BEM A2-275-mg tablets and RZR 90-mg capsules as reference (treatment A). A total of 44 subjects, 20 in Part 1 and 24 in Part 2, were enrolled. Mean (SD) age was 37.2 (11.3) years; subjects in Part 1 were a mean of 5.2 years older than subjects in Cohort 2.

In Part 2, subjects received a single oral dose of 550 mg BEM/180 mg RZR as 2×FDC2A tablets under fasting (treatment B) and fed (treatment C) conditions according to a crossover design in periods 1 and 3. In period 2, subjects received the individual reference formulations after consuming an HFHC meal or under fasting conditions with famotidine. In period 4, subjects received under fasting conditions a single oral dose of BEM 550 mg/RZR 180 mg as 2×FDC2A tablets simultaneously with famotidine (treatment D) or approximately 2 hours after famotidine (treatment E).

As summarized in the table below, FDC2A showed a high Rba (GMR B vs. A) of 1.15, 0.83 and 0.90 for RZR, BEM and Compound 1-7 based on AUCinf, respectively. Moreover, when FDC2A was administered with a standard HFHC test meal, plasma exposure of RZR slightly increased (GMR C vs. B=1.38 based on AUCinf), while plasma exposure of BEM (GMR 1.11) and Compound 1-2 (GMR 1.11) remained mostly unaffected.

The PK of BEM, its metabolites and RZR were not affected when FDC2A was simultaneously co-administered with famotidine with GMR D vs. B ranging from 0.93 to 1.13 based on AUCinf. When FDC2A was administered 2 h after famotidine, plasma exposure of RZR and BEM decreased by 35% and 30% (E vs. D) based on AUCinf, respectively, whereas plasma exposure of Compound 1-7 remained essentially unaffected (GMR 0.94).

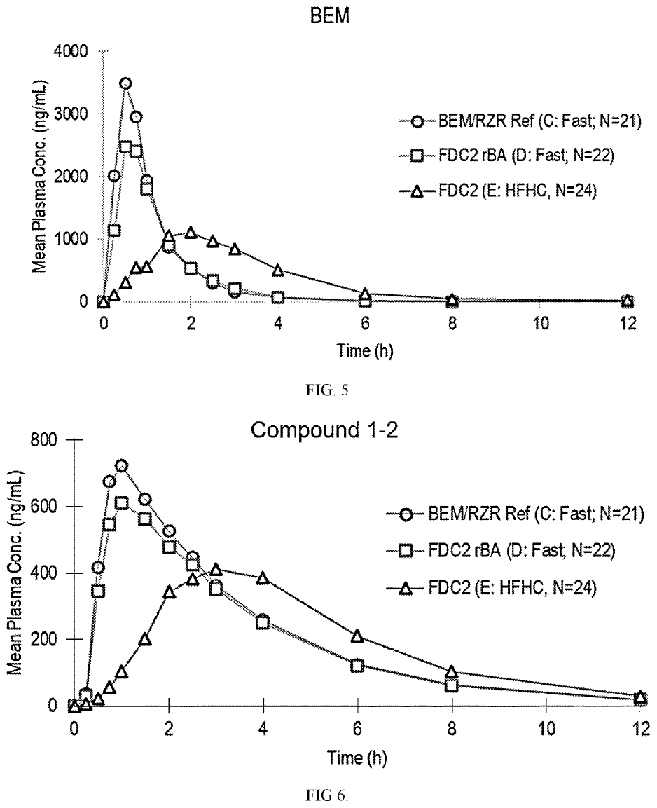

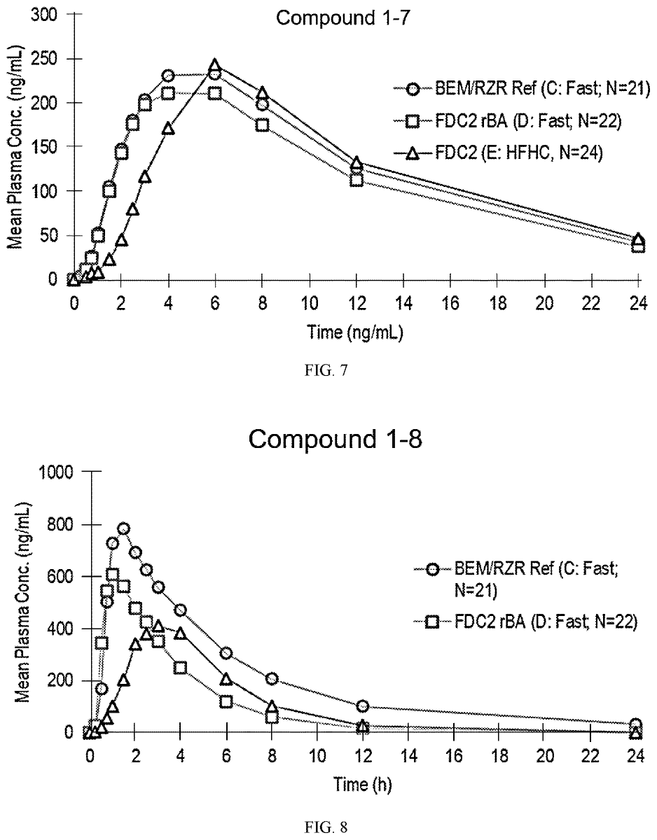

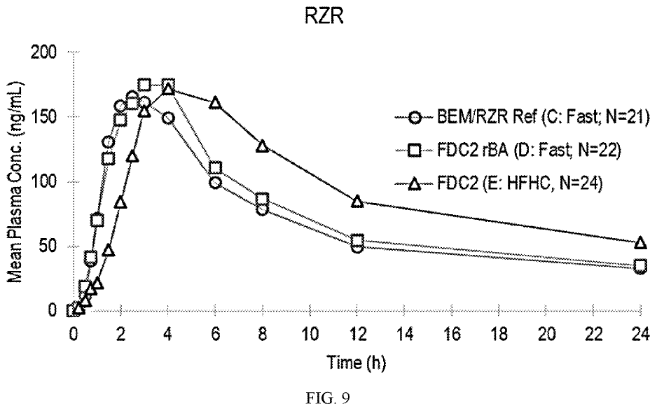

Pharmacokinetic plots can be found in FIGS. 5, 6, 7, 8, and 9.

| | BEM Tablet/ | | | | |
|---|---|---|---|---|---|
| | RZR Capsule | BEM/RZR FDC2A | | | |
| | Reference | Rba | HFHC Meal | Famotidine | Famotidine 2 h |
| Parameter | (A, N = 21) | (B, N = 22) | (C, N = 24) | (D, N = 12) | (E, N = 10) |

Compound 1

| | | | | | |
|---|---|---|---|---|---|
| Cₘₐₓ (ng/Ml) | 4293 ± 1477 | 3347 ± 1232 | 1933 ± 1339 | 3491 ± 1234 | 2511 ± 759 |
| GMR (90% CI), | NA | 0.76 | 0.53 | 1.07 | 0.79 |
| no effect = 1 | | (0.62-0.94) | (0.43-0.66) | (0.85-1.35) | (0.61-1.01) |
| Tₘₐₓ (h) | 0.5 | 0.5 | 2.0 | 0.5 | 0.5 |
| | (0.3-1.0) | (0.3-1.0) | (0.8-6.0) | (0.5-1.0) | (0.3-1.5) |
| AUC_{inf} | 3841 ± 1439 | 3268 ± 1539 | 3567 ± 1715 | 3521 ± 1319 | 2235 ± 990 |
| (ng/mL × h) | | | | | |
| GMR (90% CI), | NA | 0.83 | 1.11 | 1.11 | 0.70 |
| no effect = 1 | | (0.67-1.03) | (0.98-1.26) | (0.87-1.42) | (0.54-0.91) |

Compound 1-2

| | | | | | |
|---|---|---|---|---|---|
| Cₘₐₓ (ng/Ml) | 789 ± 346 | 713 ± 200 | 528 ± 233 | 611 ± 168 | 489 ± 153 |
| GMR (90% CI), | NA | 0.96 | 0.70 | 0.85 | 0.68 |
| no effect = 1 | | (0.78-1.17) | (0.6-0.82) | (0.71-1.03) | (0.56-0.84) |
| Tₘₐₓ (h) | 1.0 | 1.0 | 3.0 | 1.0 | 0.8 |
| | (0.8-1.5) | (0.5-4.0) | (1.5-8.0) | (0.8-2.0) | (0.5-3.0) |
| AUC_{inf} | 2512 ± 964 | 2322 ± 645 | 2235 ± 849 | 2167 ± 610 | 1596 ± 418 |
| (ng/mL × h) | | | | | |
| GMR (90% CI), | NA | 0.96 | 0.93 | 0.93 | 0.69 |
| no effect = 1 | | (0.81-1.14) | (0.84-1.04) | (0.79-1.09) | (0.58-0.82) |

Compound 1-8

| | | | | | |
|---|---|---|---|---|---|
| Cₘₐₓ (ng/Ml) | 846 ± 237 | 723 ± 279 | 385 ± 112 | 619 ± 217 | 689 ± 269 |
| GMR (90% CI), | NA | 0.84 | 0.53 | 0.85 | 0.95 |
| no effect = 1 | | (0.72-0.98) | (0.45-0.63) | (0.69-1.05) | (0.76-1.18) |
| Tₘₐₓ (h) | 1.5 | 1.5 | 3.5 | 1.5 | 2.0 |
| | (0.8-2.5) | (1.0-4.0) | (2.0-24) | (1.0-3.0) | (1.0-4.0) |
| AUC_{inf} | 5578 ± 1048 | 5113 ± 1416 | 3582 ± 920 | 5156 ± 1352 | 5844 ± 1356 |
| (ng/mL × h) | | | | | |
| GMR (90% CI), | NA | 0.90 | 0.71 | 1.01 | 1.16 |
| no effect = 1 | | (0.80-1.02) | (0.66-0.77) | (0.86-1.18) | (0.98-1.36) |

Compound 1-7

| | | | | | |
|---|---|---|---|---|---|
| Cₘₐₓ (ng/Ml) | 246 ± 61.4 | 229 ± 53.2 | 253 ± 56.9 | 237 ± 58.1 | 197 ± 63.8 |
| GMR (90% CI), | NA | 0.93 | 1.10 | 1.04 | 0.85 |
| no effect = 1 | | (0.83-1.05) | (0.99-1.23) | (0.90-1.20) | (0.72-0.99) |
| Tₘₐₓ (h) | 4.0 | 4.0 | 6.0 | 6.0 | 4.0 |
| | (2.5-8.0) | (2.5-8.0) | (4.0-8.0) | (3.0-6.0) | (3.0-8.0) |
| AUC_{inf} | 3986 ± 963 | 3611 ± 959 | 3930 ± 885 | 4026 ± 827 | 3366 ± 882 |
| (ng/mL × h) | | | | | |
| GMR (90% CI), | NA | 0.90 | 1.11 | 1.13 | 0.94 |
| no effect = 1 | | (0.79-1.02) | (1.04-1.20) | (0.98-1.31) | (0.8-1.09) |

Ruzasvir

| | | | | | |
|---|---|---|---|---|---|
| Cₘₐₓ (ng/Ml) | 180 ± 92.4 | 194 ± 82.1 | 207 ± 71.3 | 191 ± 82.7 | 116 ± 68.5 |
| GMR (90% CI), | NA | 1.15 | 1.12 | 0.99 | 0.57 |
| no effect = 1 | | (0.84-1.57) | (0.91-1.36) | (0.72-1.37) | (0.41-0.81) |
| Tₘₐₓ (h) | 2.5 | 3.5 | 4.0 | 4.0 | 4.0 |
| | (1.5-4.0) | (2.0-4.0) | (2.0-12) | (2.5-4.0) | (2.0-6.0) |
| AUC_{inf} | 2690 ± 1317 | 2952 ± 1290 | 3925 ± 1770 | 3291 ± 1661 | 2073 ± 1232 |
| (ng/mL × h) | | | | | |
| GMR (90% CI), | NA | 1.15 | 1.38 | 1.11 | 0.65 |
| no effect = 1 | | (0.86-1.53) | (1.19-1.60) | (0.80-1.53) | (0.46-0.92) |

PK parameters are presented as mean ± SD except for Tₘₐₓ where median (min-max) was shown.
NA, not applicable

### Example 15. Long Term Stability and Dissolution Study

Fixed-dose combination tablets were stored at 25 °C and 60% relative humidity for 9 months and the concentration of the active pharmaceutical compounds were assayed. The stability study confirmed that the compounds in the advantageous pharmaceutical composition are stable over a long period of time. The dissolution study confirmed that the compounds in the advantageous pharmaceutical composition maintain their beneficial dissolution profile even after extended storage.

Stability of Active Compounds In Pharmaceutical Composition

| Compound Name | 0 Months | 9 Months |
|---|---|---|
| Bemnifosbuvir | 101.6% | 100.9% |
| Ruzasvir | 100.7% | 99.1% |
| Bemnifosbuvir degradation products | ND | ND |
| Ruzasvir degradation products | ND | ND |

Initial Dissolution Study (0 months)

| | Bemnifosbuvir | Ruzasvir |
|---|---|---|
| | % Dissolution | % Dissolution |
| Timepoint | (average) | (average) |
| 10 minutes | 64 | 58 |
| 15 minutes | 78 | 78 |
| 30 minutes | 94 | 96 |
| 45 minutes | 100 | 101 |
| 60 minutes | 102 | 102 |

Dissolution After 9 Months Storage at 25 °C 60% R.H.

| | Bemnifosbuvir | Ruzasvir |
|---|---|---|
| | % Dissolution | % Dissolution |
| Timepoint | (average) | (average) |
| 10 minutes | 64 | 55 |
| 15 minutes | 81 | 79 |
| 30 minutes | 98 | 98 |
| 45 minutes | 103 | 102 |
| 60 minutes | 104 | 103 |

This specification has been described with reference to embodiments of the invention. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

We claim:

1. A fixed-dose combination comprising:
from about 250 mg to about 350 mg of (isopropyl ((S)-(((2R,3R,4R,5R)-5-(2-amino-6-(methylamino)-9H-purin-9-yl)-4-fluoro-3-hydroxy-4-methyltetrahydrofuran-2-yl) methoxy) (phenoxy)phosphoryl)-L alaninate hemisulfate (bemnifosbuvir);
from about 150 mg to about 250 mg of a spray-dried dispersion of ruzasvir;
from about 300 mg to about 450 mg of microcrystalline cellulose;
from about 125 mg to about 250 mg of a sugar alcohol selected from mannitol, lactose, and sorbitol;
from about 90 mg to about 140 mg of a disintegrant selected from crospovidone, croscarmellose sodium, and sodium starch glycolate;
from about 5 mg to about 35 mg of silicon dioxide;
from about 5 mg to about 35 mg of a lubricant selected from sodium stearyl fumarate magnesium stearate, and stearic acid.

2. The fixed-dose combination of claim 1, wherein the fixed-dose combination comprises bemnifosbuvir in a crystalline form characterized by at least five XRPD peaks selected from 5.2±0.2°, 7.3±0.2°, 8.9±0.2°, 13.6±0.2°, 17.0±0.2°, 19.9±0.2°, and 21.8±0.2° 2theta.

3. The fixed-dose combination of claim 1, wherein the fixed-dose combination comprises about 300 mg of bemnifosbuvir, in a crystalline form characterized by at least five XRPD peaks selected from 5.2±0.2°, 7.3±0.2°, 8.9±0.2°, 13.6±0.2°, 17.0±0.2°, 19.9±0.2°, and 21.8±0.2° 2theta.

4. The fixed-dose combination of claim 1, wherein the spray-dried dispersion of ruzasvir comprises ruzasvir and a hydroxyalkylcellulose.

5. The fixed-dose combination of claim 4, wherein the hydroxyalkylcellulose is HPMCAS.

6. The fixed-dose combination of claim 4, wherein the hydroxyalkylcellulose is HPMCAS-L.

7. The fixed-dose combination of claim 4, wherein the spray-dried dispersion comprises about from about 40% to about 60% ruzasvir.

8. The fixed-dose combination of claim 4, wherein the spray-dried dispersion comprises about 50% ruzasvir and about 50% HPMCAS.

9. The fixed-dose combination of claim 1, wherein the fixed-dose combination comprises about 90 mg of ruzasvir.

10. The fixed-dose combination of claim 1, wherein the fixed-dose combination comprises about 150 mg to about 250 mg of a spray-dried dispersion comprising about 50% ruzasvir and about 50% HPMCAS.

11. The fixed-dose combination of claim 1, wherein the fixed-dose combination comprises about 180 mg of a spray-dried dispersion comprising about 50% ruzasvir and about 50% HPMCAS.

12. The fixed-dose combination of claim 1, wherein the sugar alcohol is mannitol.

13. The fixed-dose combination of claim 1, wherein the sugar alcohol is lactose.

14. The fixed-dose combination of claim 1, wherein the disintegrant is crospovidone.

15. The fixed-dose combination of claim 1, wherein the lubricant is sodium stearyl fumarate.

16. The fixed-dose combination of claim 1, comprising:
from about 250 mg to about 350 mg of bemnifosbuvir, in a crystalline form characterized by at least five XRPD peaks selected from 5.2±0.2°, 7.3±0.2°, 8.9±0.2°, 13.6±0.2°, 17.0±0.2°, 19.9±0.2°, and 21.8±0.2° 2theta;
from about 150 mg to about 250 mg of a spray-dried dispersion comprising about 50% ruzasvir and about 50% HPMCAS-L;
from about 300 mg to about 450 mg of microcrystalline cellulose;
from about 125 mg to about 250 mg of sugar alcohol;
from about 90 mg to about 140 mg of crospovidone;
from about 5 mg to about 35 mg of silicon dioxide;
from about 5 mg to about 35 mg of sodium stearyl fumarate.

17. The fixed-dose combination of claim 1, comprising:
from about 290 mg to about 310 mg of bemnifosbuvir, in a crystalline form characterized by at least five XRPD peaks selected from 5.2±0.2°, 7.3±0.2°, 8.9±0.2°, 13.6±0.2°, 17.0±0.2°, 19.9±0.2°, and 21.8±0.2° 2theta;
from about 175 mg to about 185 mg of a spray-dried dispersion comprising about 50% ruzasvir and about 50% HPMCAS;
from about 340 mg to about 350 mg of microcrystalline cellulose;
from about 170 mg to about 180 mg of sugar alcohol;
from about 87 mg to about 95 mg of crospovidone;
from about 10 mg to about 15 mg of silicon dioxide;
from about 10 mg to about 15 mg of sodium stearyl fumarate.

18. A fixed-dose combination comprising an intragranular mix and an extragranular mix, wherein the intragranular mix comprises:
from about 15% to about 30% bemnifosbuvir;
from about 5% to about 25% spray-dried dispersion of ruzasvir;
from about 15% to about 30% microcrystalline cellulose;
from about 7% to about 20% mannitol;
from about 5% to about 15% crospovidone;
from about 0.5% to about 5% silicon dioxide;
from about 0.5% to about 5% sodium stearyl fumarate; and
from about 3% to about 12% sodium chloride;
in proportions selected from the ranges such that the total is 100%; and the extragranular mix comprises one or more excipients selected from a disintegrant, a glidant, and a lubricant.

19. The fixed-dose combination of claim 18, wherein the bemnifosbuvir is in a crystalline form characterized by at least five XRPD peaks selected from 5.2±0.2°, 7.3±0.2°, 8.9±0.2°, 13.6±0.2°, 17.0±0.2°, 19.9±0.2°, and 21.8±0.2° 2theta.

20. The fixed-dose combination of claim 18, wherein the spray-dried dispersion of ruzasvir comprises ruzasvir and a hydroxyalkylcellulose.

21. The fixed-dose combination of claim 20, wherein the hydroxyalkylcellulose is HPMCAS.

22. The fixed-dose combination of claim 20, wherein the hydroxyalkylcellulose is HPMCAS-L.

23. The fixed-dose combination of claim 20, wherein the spray-dried dispersion comprises about from about 40% to about 60% ruzasvir.

24. The fixed-dose combination of claim 20, wherein the spray-dried dispersion comprises about 50% ruzasvir and about 50% HPMCAS.

25. The fixed-dose combination of claim 18, wherein the intragranular mix comprises:
from about 15% to about 30% bemnifosbuvir, in a crystalline form;
from about 5% to about 25% spray-dried dispersion comprising about 50% ruzasvir and about 50% HPMCAS-L;
from about 15% to about 30% microcrystalline cellulose;
from about 7% to about 20% mannitol;
from about 5% to about 15% crospovidone;
from about 0.5% to about 5% silicon dioxide;
from about 0.5% to about 5% sodium stearyl fumarate; and
from about 3% to about 12% sodium chloride;
in proportions selected from the ranges such that the total intragranular components is from about 90% to about 99%;
and the extragranular mix comprises:
from about 0.5% to about 5% crospovidone;
from about 0.05% to about 2% silicon dioxide; and
from about 0.05% to about 2% sodium stearyl fumarate;
in proportions selected from the ranges such that the total extragranular mix is from about 1% to about 10%; and
in proportions selected from the ranges such that the total of Intragranular and Extragranular components is 100%.

26. A fixed-dose combination comprising an intragranular mix and an extragranular mix, wherein the intragranular mix comprises:
about 300 mg of bemnifosbuvir, in a crystalline form characterized by at least five XRPD peaks selected from 5.2±0.2°, 7.3±0.2°, 8.9±0.2°, 13.6±0.2°, 17.0±0.2°, 19.9±0.2°, and 21.8±0.2° 2theta;
about 180 mg of a spray-dried dispersion comprising about 50% ruzasvir and about 50% HPMCAS;
about 350 mg of microcrystalline cellulose;
about 175 mg of mannitol;
about 90 mg of crospovidone;
about 12 mg of silicon dioxide;
about 12 mg of sodium stearyl fumarate; and
about 90 mg of sodium chloride;
and the extragranular mix comprises:
about 25 mg crospovidone;
about 6 mg silicon dioxide; and
about 6 mg sodium stearyl fumarate.

27. The fixed-dose combination of claim 1, wherein the combination further comprises a coating.

28. The fixed-dose combination of claim 18, wherein the combination further comprises a coating.

29. The fixed-dose combination of claim 1, wherein the combination is coated with a film coating.

30. The fixed-dose combination of claim 18, wherein the combination is coated with a film coating.
